(19) United States
(12) Patent Application Publication
(10) Pub. No.: US 2026/0242497 A1
(43) Pub. Date: Aug. 20, 2026

(54) ANTIBODIES AND POLYPEPTIDES COMPRISING VARIANT FC REGIONS

(71) Applicant: Vial Health Technology Inc., San Francisco, CA (US)

(72) Inventors: Joshua PASCOE, Toronto (CA); Ritu RANDHAWA, Hamilton, CA (US); Sonam BHUPTANI, Fremont, CA (US)

(21) Appl. No.: 19/181,244

(22) Filed: Apr. 16, 2025

Related U.S. Application Data
(60) Provisional application No. 63/635,023, filed on Apr. 17, 2024.
(60) Provisional application No. 63/743,892, filed on Jan. 10, 2025.

(51) Int. Cl. C07K 16/28; A61K 47/68; A61P 29/00; C07K 16/24

(52) U.S. Cl. CPC C07K 16/2875; A61K 47/6879; A61P 29/00; C07K 16/244; C07K 16/245; C07K 2317/24; C07K 2317/31; C07K 2317/52; C07K 2317/565; C07K 2317/622; C07K 2317/76; C07K 2317/92; C07K 2317/94

(57) ABSTRACT

Disclosed herein are antibodies and antigen-binding fragments thereof that bind target antigens (e.g., tumor necrosis factor-like ligand 1A (TL1A)) and polypeptides comprising variant Fc regions.

### CROSS-REFERENCE TO RELATED APPLICATIONS

This application claims the benefit of U.S. Provisional Patent Application No. 63/635,023 filed Apr. 17, 2024, and U.S. Provisional Application No. 63/743,892 filed Jan. 10, 2025, the entire disclosures of each of which are incorporated herein by reference in their entirety.

### REFERENCE TO AN ELECTRONIC SEQUENCE LISTING

The Sequence Listing XML associated with this application is provided in XML file format and is hereby incorporated by reference into the specification. The name of the XML file containing the Sequence Listing XML is VIAL_001_02US_SeqList_ST26.xml. The XML file is 142,118 bytes, and created on Apr. 14, 2025, and is being submitted electronically via USPTO Patent Center.

### TECHNICAL FIELD

Disclosed herein are antibodies and antigen-binding fragments thereof that bind target antigens (e.g., tumor necrosis factor-like ligand 1A (TL1A)), and polypeptides comprising variant Fc regions.

### BACKGROUND

Antibodies capable of binding certain target antigens can, in some contexts and examples, provide candidate therapeutics for treatment of diseases associated with those target antigens. For example, antibodies capable of binding cytokines associated with inflammatory diseases and/or autoimmune conditions may be potential therapeutics for those diseases and/or conditions.

Tumor necrosis factor-like ligand 1A (TL1A), also known as TNFSF15, is a member of the tumor necrosis factor family and is expressed in different immune cells, including monocytes, macrophages, dendritic cells, and T cells, as well as in various non-immune cells. TL1A has been shown to be abnormally expressed in autoimmune diseases, including rheumatoid arthritis, inflammatory bowel disease, psoriasis, primary biliary cirrhosis, systemic lupus erythematosus, and ankylosing spondylitis. Interleukin 13 (IL1P) is a pro-inflammatory cytokine that plays a key role in innate immune responses. IL1P is associated with diseases and conditions including rheumatoid arthritis, sepsis, and autoimmune conditions. Interleukin 13 (IL13) is a cytokine that is involved in regulating immune responses. IL13 is associated with diseases and conditions including allergic inflammation, asthma, and atopic dermatitis. Thymic Stromal Lymphopoietin (TSLP) is an epithelial cell-derived cytokine involved in initiating immune responses. TSLP is associated with diseases and conditions including allergic inflammation, asthma, and atopic dermatitis. Interleukin 15 (IL15) is a cytokine that is involved in inflammatory immune responses and modulation of innate and adaptive immune cells. IL15 is associated with diseases and conditions including rheumatoid arthritis, psoriasis, and inflammatory bowel disease. There is a need for therapeutics that target such autoimmune diseases. Therapeutic antibodies are limited by certain pharmacokinetic properties, including the half-life of the antibody within the body. Antibody half-life directly affects the required dosing interval for therapeutic antibodies. One factor that affects antibody half-life is the affinity with which the Fc region of the antibody binds the neonatal Fc receptor (FcRn). There is a need for variant Fc domains with improved pharmacokinetic properties, including increased half-life.

### SUMMARY

Disclosed herein are polypeptides comprising a variant IgG Fc region, wherein the variant IgG Fc region comprises amino acid substitutions at: a) position Q311 and/or M428; and b) position H433 and/or N434; relative to a wild-type IgG Fc region, wherein numbering is according to the EU index as in Kabat.

In some embodiments, the variant IgG Fc region comprises amino acid substitutions at: a) Q311 and H433; b) M428 and N434; c) Q311 and N434; d) M428 and H433; e) Q311, M428, and H433; f) Q311, M428, and N434; g) Q311, H433, and N434; or h) M428, H433, and N434.

In some embodiments, the variant IgG Fc variant IgG Fc region comprises amino acid substitutions at Q311, M428, and N434. In some embodiments, the variant IgG Fc region comprises amino acid substitutions at M428, and H433, and N434.

In some embodiments, the variant IgG Fc region comprises amino acid substitutions at Q311, M428, H433, and N434.

In some embodiments, the amino acid substitutions comprise: a) Q311R and H433K; b) M428L and N434F; c) Q311R and N434F; d) M428L and H433K; e) Q311R, M428L, and H433K; f) Q311R, M428L, and N434F; g) Q311R, H433K, and N434F; or h) M428L, H433K, and N434F.

In some embodiments, the amino acid substitutions comprise Q311R, M428L, and N434F. In some embodiments, the amino acid substitutions comprise M428L, H433K, and N434F.

In some embodiments, the amino acid substitutions comprise Q311R, M428L, H433K, and N434F.

In some embodiments, the variant IgG Fc region further comprises one or more amino acid substitutions selected from the group consisting of L234A, L235A, and G237A. In some embodiments, the variant IgG Fc region is an IgG1 Fc region. In some embodiments, the variant IgG Fc region is an IgG4 Fc region. In some embodiments, the variant IgG Fc region is a human IgG Fc region.

In some embodiments, the polypeptide comprising a variant IgG Fc region has increased affinity for FcRn compared to an IgG having a wild-type IgG Fc region. In some embodiments, the polypeptide comprising a variant IgG Fc region has increased affinity for FcRn compared to an IgG having a reference IgG Fc region comprising the amino acid sequence of SEQ ID NOs: 42, 107, 108, 109, or 110.

In some embodiments, the polypeptide comprising a variant IgG Fc region has an increased half-life compared to the half-life of an IgG having a wild-type IgG Fc region. In some embodiments, the polypeptide comprising a variant IgG Fc region has an increased half-life compared to the half-life of an IgG having a reference IgG Fc region comprising the amino acid sequence of SEQ ID NOs: 42, 107, 108, 109, or 110.

In some embodiments, the variant IgG Fc region comprises the amino acid sequence of any one of SEQ ID NOs: 40, 41, 99-102, and 133-137.

In some embodiments, the polypeptide comprises an antibody or antigen-binding fragment thereof. In some embodiments, the polypeptide is an antibody. In some embodiments, the antibody is a human antibody, a humanized antibody, or monoclonal antibody.

In some embodiments, the antibody comprises a heavy chain constant region comprising any one of SEQ ID NOs: 33-35, 95-98, and 128-132.

In some embodiments, the antibody or antigen-binding fragment thereof binds tumor necrosis factor-like ligand 1A (TL1A), interleukin IL1P, IL13, IL15, or TSLP.

In some embodiments, the antibody comprises two or more antigen binding regions. In some embodiments, one of the two or more binding regions binds IL13 or TSLP. In some embodiments, the antibody comprises a first binding region that binds IL13 and a second binding region that binds TSLP.

Also disclosed herein are polypeptides comprising a variant IgG Fc region described herein, for use as a medicament. Also disclosed herein are immunoconjugates comprising a polypeptide comprising a variant IgG Fc region disclosed herein, pharmaceutical compositions comprising a polypeptide comprising a variant IgG Fc region disclosed herein disclosed herein, nucleic acid molecules encoding a polypeptide comprising a variant IgG Fc region disclosed herein disclosed herein, vectors comprising the nucleic acid molecules disclosed herein, and recombinant cells comprising a polypeptide comprising a variant IgG Fc region, nucleic acid molecule, and/or vector described herein. Also disclosed herein are methods of producing a polypeptide comprising a variant IgG Fc region disclosed herein, wherein the polypeptide is a recombinant polypeptide comprising a variant Fc, and wherein the recombinant polypeptide is produced using a recombinant vector in a recombinant cell.

Also disclosed herein are antibodies or antigen-binding fragments thereof that bind tumor necrosis factor-like ligand 1A (TL1A). In some embodiments, the antibodies or antigen-binding fragment thereof comprises: a) a heavy chain variable region (VH) with at least 90% sequence identity to SEQ ID NO: 7 and a light chain variable region (VL) comprising an amino acid sequence with at least 90% sequence identity to SEQ ID NO: 8; b) a VH comprising an amino acid sequence with at least 90% sequence identity to SEQ ID NO: 13 and a VL comprising an amino acid sequence with at least 90% sequence identity to SEQ ID NO: 14; c) a VH comprising an amino acid sequence with at least 90% sequence identity to SEQ ID NO: 7 and a VL comprising an amino acid sequence with at least 90% sequence identity to SEQ ID NO: 18; d) a VH comprising an amino acid sequence with at least 90% sequence identity to SEQ ID NO: 7 and a VL comprising an amino acid sequence with at least 90% sequence identity to SEQ ID NO: 20; e) a VH comprising an amino acid sequence with at least 90% sequence identity to SEQ ID NO: 23 and a VL comprising an amino acid sequence with at least 90% sequence identity to SEQ ID NO: 20; or f) a VH comprising an amino acid sequence with at least 90% sequence identity to SEQ ID NO: 28 and a VL comprising an amino acid sequence with at least 90% sequence identity to SEQ ID NO: 20.

In some embodiments, the antibody or antigen-binding fragment thereof comprises: a) a heavy chain complementarity determining region 1 (HCDR1) having at least 90% sequence identity with SEQ ID NO: 1, a HCDR2 having at least 90% sequence identity with SEQ ID NO: 2, a HCDR3 having at least 90% sequence identity with SEQ ID NO: 3, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6; b) a HCDR1 having at least 90% sequence identity with SEQ ID NO: 1, a HCDR2 having at least 90% sequence identity with SEQ ID NO: 11, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 5, and a LCDR3 having at least 90% sequence identity with SEQ ID NO: 12; c) a HCDR1 having at least 90% sequence identity with SEQ ID NO: 1, a HCDR2 having at least 90% sequence identity with SEQ ID NO: 2, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 6; d) a HCDR1 having at least 90% sequence identity with SEQ ID NO: 1, a HCDR2 having at least 90% sequence identity with SEQ ID NO: 2, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 12; e) a HCDR1 having at least 90% sequence identity with SEQ ID NO: 1, a HCDR2 having at least 90% sequence identity with SEQ ID NO: 22, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 12; f) a HCDR1 having at least 90% sequence identity with SEQ ID NO: 25, a HCDR2 having at least 90% sequence identity with SEQ ID NO: 2, a HCDR3 having at least 90% sequence identity with SEQ ID NO: 3, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6; or g) a HCDR1 having at least 90% sequence identity with SEQ ID NO: 25, a HCDR2 having at least 90% sequence identity with SEQ ID NO: 2, a HCDR3 having at least 90% sequence identity with SEQ ID NO: 27, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6.

In some embodiments, the antibody or antigen-binding fragment thereof comprises: a) a heavy chain variable region (VH) comprising the amino acid sequence of SEQ ID NO: 7; and a light chain variable region (VL) comprising the amino acid sequence of SEQ ID NO: 8; b) a VH comprising the amino acid sequence of SEQ ID NO: 13 and a VL comprising the amino acid sequence of SEQ ID NO: 14; c) a VH comprising the amino acid sequence of SEQ ID NO: 7 and a VL comprising the amino acid sequence of SEQ ID NO: 18; d) a VH comprising the amino acid sequence of SEQ ID NO: 7 and a VL comprising the amino acid sequence of SEQ ID NO: 20; e) a VH comprising the amino acid sequence of SEQ ID NO: 23 and a VL comprising the amino acid sequence of SEQ ID NO: 20; or f) a VH comprising the amino acid sequence of SEQ ID NO: 28 and a VL comprising the amino acid sequence of SEQ ID NO: 20.

Also disclosed herein are antibodies or antigen-binding fragments thereof that bind interleukin-1β (IL1β). In some embodiments, the antibody or antigen-binding fragment thereof comprises a heavy chain variable region (VH) comprising an amino acid sequence with at least 90% sequence identity to SEQ ID NO: 49 and a light chain variable region (VL) comprising an amino acid sequence with at least 90% sequence identity to SEQ ID NO: 50.

In some embodiments, the antibody or antigen-binding fragment thereof comprises a heavy chain complementarity determining region 1 (HCDR1) having at least 90% sequence identity with SEQ ID NO: 43, a HCDR2 having at least 90% sequence identity with SEQ ID NO: 44, a HCDR3 having at least 90% sequence identity with SEQ ID NO: 45, a light chain complementarity determining region 1 (LCDR1) having at least 90% sequence identity with SEQ ID NO: 46, a LCDR2 having at least 90% sequence identity with SEQ ID NO: 47, and a LCDR3 having at least 90% sequence identity with SEQ ID NO: 48.

In some embodiments, the antibody or antigen-binding fragment thereof comprises a heavy chain variable region (VH) comprising the amino acid sequence of SEQ ID NO: 49 and a light chain variable region (VL) comprising the amino acid sequence of SEQ ID NO: 50.

Also disclosed herein are antibodies or antigen-binding fragments thereof that bind interleukin-13 (IL13). In some embodiments, the antibody or antigen-binding fragment thereof comprises: a) a heavy chain variable region (VH) comprising an amino acid sequence with at least 90% sequence identity to SEQ ID NO: 59 and a light chain variable region (VL) comprising an amino acid sequence with at least 90% sequence identity to SEQ ID NO: 60; b) a VH comprising an amino acid sequence with at least 90% sequence identity to SEQ ID NO: 64 and a VL comprising an amino acid sequence with at least 90% sequence identity to SEQ ID NO: 60; or c) a VH comprising an amino acid sequence with at least 90% sequence identity to SEQ ID NO: 86 and a VL comprising an amino acid sequence with at least 90% sequence identity to SEQ ID NO: 87.

In some embodiments, the antibody or antigen-binding fragment thereof comprises: a) a heavy chain complementarity determining region 1 (HCDR1) having at least 90% sequence identity with SEQ ID NO: 53, a HCDR2 having at least 90% sequence identity with SEQ ID NO: 54, a HCDR3 having at least 90% sequence identity with SEQ ID NO: 55, a light chain complementarity determining region 1 (LCDR1) having at least 90% sequence identity with SEQ ID NO: 56, a LCDR2 having at least 90% sequence identity with SEQ ID NO: 57, and a LCDR3 having at least 90% sequence identity with SEQ ID NO: 58; b) a HCDR1 having at least 90% sequence identity with SEQ ID NO: 53, a HCDR2 having at least 90% sequence identity with SEQ ID NO: 54, a HCDR3 having at least 90% sequence identity with SEQ ID NO: 63, a LCDR1 having at least 90% sequence identity with SEQ ID NO: 56, a LCDR2 having at least 90% sequence identity with SEQ ID NO: 57, and a LCDR3 having at least 90% sequence identity with SEQ ID NO: 58; or c) a HCDR1 having at least 90% sequence identity with SEQ ID NO: 53, a HCDR2 having at least 90% sequence identity with SEQ ID NO: 54, a HCDR3 having at least 90% sequence identity with SEQ ID NO: 82, a LCDR1 having at least 90% sequence identity with SEQ ID NO: 56, a LCDR2 having at least 90% sequence identity with SEQ ID NO: 57, and a LCDR3 having at least 90% sequence identity with SEQ ID NO: 58.

In some embodiments, the antibody or antigen-binding fragment thereof comprise: a) a heavy chain variable region (VH) comprising the amino acid sequence of SEQ ID NO: 59 and a light chain variable region (VL) comprising the amino acid sequence of SEQ ID NO: 60; b) a VH comprising the amino acid sequence of SEQ ID NO: 64 and a VL comprising the amino acid sequence of SEQ ID NO: 60; or c) a VH comprising the amino acid sequence of SEQ ID NO: 86 and a VL comprising the amino acid sequence of SEQ ID NO: 87.

Also disclosed herein are antibodies or antigen-binding fragments thereof that bind interleukin-15 (IL15). In some embodiments, the antibody or antigen-binding fragment thereof comprises a heavy chain variable region (VH) comprising an amino acid sequence with at least 90% sequence identity to SEQ ID NO: 72 and a light chain variable region (VL) comprising an amino acid sequence with at least 90% sequence identity to SEQ ID NO: 73.

In some embodiments, the antibody or antigen-binding fragment thereof comprises a heavy chain complementarity determining region 1 (HCDR1) having at least 90% sequence identity with SEQ ID NO: 66, a HCDR2 having at least 90% sequence identity with SEQ ID NO: 67, a HCDR3 having at least 90% sequence identity with SEQ ID NO: 68, a light chain complementarity determining region 1 (LCDR1) having at least 90% sequence identity with SEQ ID NO: 69, a LCDR2 having at least 90% sequence identity with SEQ ID NO: 70, and a LCDR3 having at least 90% sequence identity with SEQ ID NO: 71.

In some embodiments, the antibody or antigen-binding fragment thereof comprises a heavy chain variable region (VH) comprising the amino acid sequence of SEQ ID NO: 72 and a light chain variable region (VL) comprising the amino acid sequence of SEQ ID NO: 73.

Also disclosed herein are antibodies or antigen-binding fragments thereof that bind thymic stromal lymphopoietin (TSLP). In some embodiments, the antibody or antigen-binding fragment thereof comprises a heavy chain variable region (VH) comprising an amino acid sequence with at least 90% sequence identity to SEQ ID NO: 84 and a light chain variable region (VL) comprising an amino acid sequence with at least 90% sequence identity to SEQ ID NO: 85.

In some embodiments, the antibody or antigen-binding fragment thereof comprises a heavy chain complementarity determining region 1 (HCDR1) having at least 90% sequence identity with SEQ ID NO: 76, a HCDR2 having at least 90% sequence identity with SEQ ID NO: 77, a HCDR3 having at least 90% sequence identity with SEQ ID NO: 78, a light chain complementarity determining region 1 (LCDR1) having at least 90% sequence identity with SEQ ID NO: 79, a LCDR2 having at least 90% sequence identity with SEQ ID NO: 80, and a LCDR3 having at least 90% sequence identity with SEQ ID NO: 81.

In some embodiments, the antibody or antigen-binding fragment thereof comprises a heavy chain variable region (VH) comprising the amino acid sequence of SEQ ID NO: 84 and a light chain variable region (VL) comprising the amino acid sequence of SEQ ID NO: 85.

In some embodiments, the antibody further comprises a region capable of binding a second antigen. In some embodiments, the second antigen is thymic stromal lymphopoietin (TSLP), and the region capable of binding a second antigen comprises a heavy chain variable region (VH) comprising an amino acid sequence with at least 90% sequence identity to SEQ ID NO: 84 and a light chain variable region (VL) comprising an amino acid sequence with at least 90% sequence identity to SEQ ID NO: 85. In some embodiments, the region capable of binding a second antigen comprises a heavy chain complementarity determining region 1 (HCDR1) having at least 90% sequence identity with SEQ ID NO: 76, a HCDR2 having at least 90% sequence identity with SEQ ID NO: 77, a HCDR3 having at least 90% sequence identity with SEQ ID NO: 78, a light chain complementarity determining region 1 (LCDR1) having at least 90% sequence identity with SEQ ID NO: 79, a LCDR2 having at least 90% sequence identity with SEQ ID NO: 80, and a LCDR3 having at least 90% sequence identity with SEQ ID NO: 81. In some embodiments, the region capable of binding a second antigen comprises a heavy chain variable region (VH) comprising the amino acid sequence of SEQ ID NO: 84 and a light chain variable region (VL) comprising the amino acid sequence of SEQ ID NO: 85.

In some embodiments, the antibody or antigen-binding fragment thereof is humanized or chimeric. In some embodiments, the VH region, the VL region, or both the VH and the VL region comprise one or more human framework region amino acid sequences. In some embodiments, the antibody or antigen-binding portion comprises an immunoglobulin constant region. In some embodiments, the immunoglobulin constant region is an IgG, IgE, IgM, IgD, IgA or IgY constant region. In some embodiments, the immunoglobulin constant region is an IgG1, IgG2, IgG3, IgG4, IgAQ1 or IgA2 constant region. In some embodiments, the immunoglobulin constant region is immunologically inert.

In some embodiments, the antibody or antigen-binding fragment thereof comprises a human IgG1 constant region comprising a L234A substitution, a L235A substitution, a G237A substitution, a Q311R substitution, a D356E substitution, a L358M substitution, a M428L substitution, a H433K substitution, a N434S substitution, a N434F substitution or a combination thereof, wherein numbering is according to the EU index as in Kabat.

In some embodiments, the immunoglobulin constant region comprises a human IgG4 constant region comprising a F234A substitution, a L235A substitution, a G237A substitution, Q311R substitution, a D356E substitution, a L358M substitution, a M428L substitution, a H433K substitution, a N434S substitution, a N434F substitution or a combination thereof, wherein numbering is according to the EU index as in Kabat.

In some embodiments, the human IgG1 constant region or human IgG4 constant region comprises a Q311R substitution, a M428L substitution, and a N434F substitution, wherein numbering is according to the EU index as in Kabat.

In some embodiments, the human IgG1 constant region comprises a M428L substitution, a H433K substitution, and a N434F substitution, wherein numbering is according to the EU index as in Kabat.

In some embodiments, the human IgG1 constant region or human IgG4 constant region comprises a Q311R substitution, a M428L substitution, a H433K substitution, and a N434F substitution, wherein numbering is according to the EU index as in Kabat.

In some embodiments, the immunoglobulin constant region is a human IgG1 constant region comprising a L234A substitution, a L235A substitution, a G237A substitution, a D356E substitution, and a L358M substitution, wherein numbering is according to the EU index as in Kabat.

In some embodiments, the immunoglobulin constant region is a human IgG4 constant region comprising a F234A substitution, a L235A substitution, a G237A substitution, a D356E substitution, and a L358M substitution, wherein numbering is according to the EU index as in Kabat.

In some embodiments, the antibody or antigen-binding fragment thereof comprises a M428L substitution and a N434S substitution, wherein numbering is according to the EU index as in Kabat.

In some embodiments, the antibody or antigen-binding fragment thereof comprises a Q311R substitution, a M428L substitution, a H433K substitution, and a N434F substitution, wherein numbering is according to the EU index as in Kabat.

In some embodiments, the antibody or antigen-binding fragment thereof comprises: a) a heavy chain (HC) comprising the amino acid sequence of SEQ ID NO: 9 and a light chain (LC) comprising the amino acid sequence of SEQ ID NO: 10; b) a HC comprising the amino acid sequence of SEQ ID NO: 15 and a LC comprising the amino acid sequence of SEQ ID NO: 16; c) a HC comprising the amino acid sequence of SEQ ID NO: 9 and a LC comprising the amino acid sequence of SEQ ID NO: 19; d) a HC comprising the amino acid sequence of SEQ ID NO: 9 and a LC comprising the amino acid sequence of SEQ ID NO: 21; e) a HC comprising the amino acid sequence of SEQ ID NO: 24 and a LC comprising the amino acid sequence of SEQ ID NO: 21; f) a HC comprising the amino acid sequence of SEQ ID NO: 26 and a LC comprising the amino acid sequence of SEQ ID NO: 10; g) a HC comprising the amino acid sequence of SEQ ID NO: 29 and a LC comprising the amino acid sequence of SEQ ID NO: 21; h) a HC comprising the amino acid sequence of SEQ ID NO: 51 and a LC comprising the amino acid sequence of SEQ ID NO: 52; i) a HC comprising the amino acid sequence of SEQ ID NO: 61 and a LC comprising the amino acid sequence of SEQ ID NO: 62; j) a HC comprising the amino acid sequence of SEQ ID NO: 65 and a LC comprising the amino acid sequence of SEQ ID NO: 62; k) a HC comprising the amino acid sequence of SEQ ID NO: 74 and a LC comprising the amino acid sequence of SEQ ID NO: 75; l) a HC comprising the amino acid sequence of SEQ ID NO: 88 and a LC comprising the amino acid sequence of SEQ ID NO: 89; or m) a single-chain variable fragment linked to an Fc region (scFv-Fc) comprising the amino acid sequence of SEQ ID NO: 90.

In some embodiments, the antibody or antigen-binding fragment thereof comprises a heavy chain (HC) comprising the amino acid sequence of SEQ ID NO: 88 and a light chain (LC) comprising the amino acid sequence of SEQ ID NO: 89; and a single-chain variable fragment linked to an Fc region (scFv-Fc) comprising the amino acid sequence of SEQ ID NO: 90.

In some embodiments, the immunoglobulin constant region comprises (i) any one of SEQ ID NO: 34, 35, 95-98, or 128-132; and (ii) SEQ ID NO: 36. In some embodiments, the antibody has increased affinity for FcRn compared to a comparator antibody. In some embodiments, the comparator antibody is RVT-3101, AVTX-009, APG-777, APG-777×AIO-001, or CALY-002.

In some embodiments, the antibody or antigen-binding fragment is an Fab, an Fab′, an F(ab′)2, an Fv, an scFv, a maxibody, a minibody, a diabody, a triabody, a tetrabody, or a bis-scFv. In some embodiments, the antibody or antigen-binding portion is bispecific.

Also disclosed herein are immunoconjugates comprising an antibody or antigen-binding fragment thereof disclosed herein, pharmaceutical compositions comprising an antibody or antigen-binding fragment thereof disclosed herein, nucleic acid molecules encoding an antibody or antigen-binding fragment thereof disclosed herein, vectors comprising the nucleic acid molecules disclosed herein, and recombinant host cells comprising the antibodies or antigen-binding fragments thereof, the nucleic acid molecules, and/or the vectors described herein.

Also disclosed herein are methods of producing an antibody or an antigen-binding fragment thereof described herein. Also disclosed herein are methods for reducing death recenter 3 (DR3) signaling in a cell, wherein the method comprises contacting the cell with an antibody or antigen-binding portion thereof described herein. Also disclosed herein are methods for reducing IL4/IL13 signaling in a cell, wherein the method comprises contacting the cell with the antibody or antigen-binding portion described herein. Also disclosed herein are methods for reducing IL6 release by a cell, wherein the method comprises contacting the cell with the antibody or antigen-binding portion described herein. Also disclosed herein are methods for reducing release of one or more pro-inflammatory cytokines by a cell, wherein the method comprises contacting the cell with the antibody or antigen-binding portion disclosed herein. Also disclosed herein are methods for reducing IL6 release by a cell, wherein the method comprises contacting the cell with the antibody or antigen-binding portion disclosed herein. In some embodiments, the method results in reduced proliferation of the cell. In some embodiments, the method results in reduced production of a cytokine, preferably TNF-α.

Also disclosed herein are an antibody or antigen-binding portion thereof described herein, an immunoconjugate described herein, or a pharmaceutical composition described herein, for use as a medicament.

### BRIEF DESCRIPTIONS OF THE DRAWINGS

FIGS. 1A-1B provide diagrams of exemplary mutations in the variant Fc regions described herein.

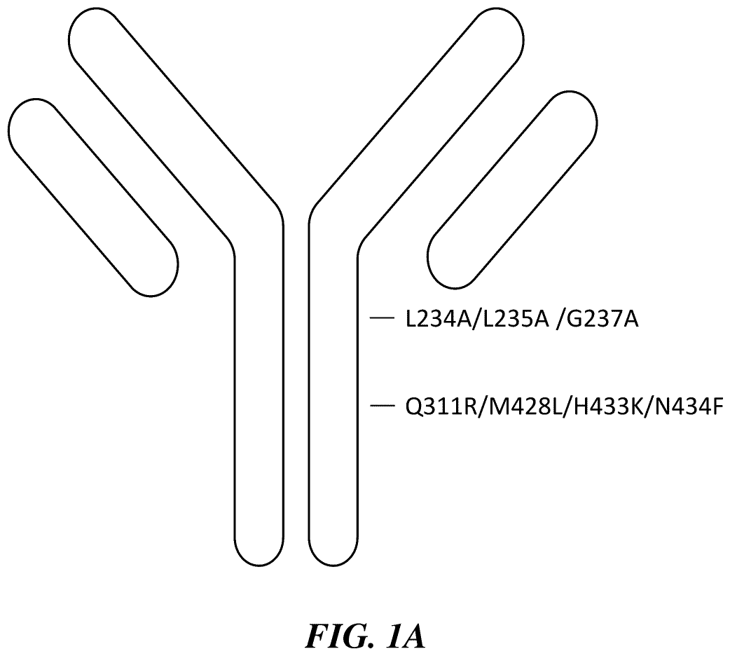

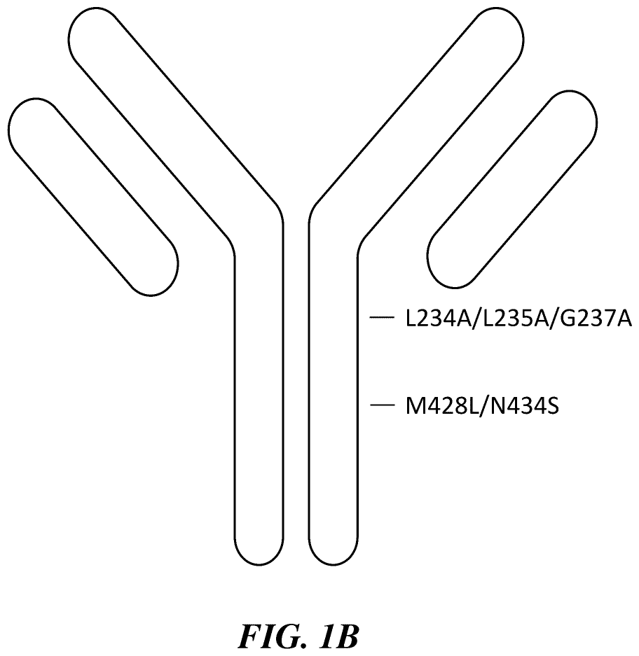

### DETAILED DESCRIPTION OF ILLUSTRATIVE EMBODIMENTS

Provided herein are antibodies and therapeutic uses of such antibodies. The antibodies disclosed herein have favorable pharmacokinetics, low immunogenicity, and high efficacy.

Tumor necrosis factor-like ligand 1A (TL1A, UniProt ID: 95150), also known as TNFSF15, is a member of the tumor necrosis factor family. TL1A activates signaling pathways that modulate inflammation and apoptosis. Binding of TL1A to its receptor death receptor 3 (DR3) can lead to interaction of DR3 with adaptor protein TNFR-associated death domain protein (TRADD), recruitment of TRAF2 and R1P1, and signaling through MAP kinases (ERK, p38, and JNK), NF-κB, and PI3K, resulting in expression of pro-inflammatory genes. TRADD can also bind Fas-associated death domain (FADD) and RIP3, leading to activation of caspase pathways (for example, caspase-3 and -7) and apoptotic cell death. TL1A can be expressed by monocytes, macrophages, dendritic cells (DCs), T cells, and non-immune cells such as synovial fibroblasts and endothelial cells. TL1A is constitutively expressed in endothelial cells and is up-regulated in response to tumor necrosis factor-α (TNF-α) stimulation. Expression of TL1A in DCs and macrophages is increased by TLR4, TLR11, or Fc region of IgG (FcTR) signaling. TL1A has been shown to be abnormally expressed in autoimmune diseases, including rheumatoid arthritis, inflammatory bowel disease, psoriasis, primary biliary cirrhosis, systemic lupus erythematosus, and ankylosing spondylitis. There is a need for therapeutics that target such autoimmune diseases. TL1A antibodies are designed to block its interaction with DR3, thereby inhibiting downstream signaling pathways associated with inflammation, offering a therapeutic approach to treating autoimmune diseases.

Interleukin 13 (IL1β, UniProt ID: 01584), also known as IL1F2 or catabolin, is a pro-inflammatory cytokine that plays a key role in innate immune responses. IL1β is produced as an inactive precursor (pro-IL1) and is activated by cleavage via caspase-1, which is part of the inflammasome complex. IL1β is secreted via non-classical secretory pathways. IL1β binds to the IL1 receptor type 1 and signals through NF-kB and MAP kinase pathways. Downstream effects of IL1β signaling include inflammation, production of IL6 and TNF-α, and promotion of Th17 differentiation of T cells. IL1β is associated with diseases and conditions including rheumatoid arthritis, sepsis, and autoimmune conditions.

Interleukin 13 (IL13, UniProt ID: P35225), also known as NC30, is a cytokine that is involved in regulating immune responses. IL13 is produced by Th2 cells, innate lymphoid cells, mast cells, and basophils. IL13 binds to a heterodimeric receptor complex consisting of IL13Rα1 and IL4Rα and signals through JAK-STAT pathways, resulting in translocation of STATs (e.g., STAT6) to the nucleus and transcription of IL13 responsive genes. Downstream effects of IL13 signaling include B-cell proliferation, activation of eosinophils, basophils, and mast cells, and control of IL33 activity and proinflammatory cytokine production. IL13 is associated with diseases and conditions including allergic inflammation, asthma, and atopic dermatitis.

Thymic Stromal Lymphopoietin (TSLP, UniProt ID: Q969D9) is an epithelial cell-derived cytokine involved in initiating immune responses. TSLP is produced by epithelial cells at barrier surfaces including skin, gut, lungs, and thymus. TSLP binds to the TSLP receptor (TSLPR), a heterodimer of the IL7 receptor alpha chain and the TSLPR chain, and signals through MAPK and JAK-STAT pathways, resulting in translocation of STATs (e.g., STAT5) to the nucleus and transcription of responsive genes. Downstream effects of TSLP signaling include Th2 polarization of T cells, T-cell proliferation, Th2 cytokine production, and B-cell expansion and differentiation. TSLP is associated with diseases and conditions including allergic inflammation, asthma, and atopic dermatitis.

Interleukin 15 (IL15, UniProt ID: P40933) is a cytokine that is involved in inflammatory immune responses and modulation of innate and adaptive immune cells. IL15 is produced by monocytes and dendritic cells, and can be secreted and/or expressed as a membrane-bound complex. IL15 binds to a receptor complex comprising IL15Rα, IL2/15Rβ (CD122), and the common gamma chain (CD132), and signals through JAK-STAT pathways and PI3K, resulting in translocation of STATs (e.g., STAT3 and STAT5) to the nucleus and transcription of responsive genes. Downstream effects of IL15 signaling include stimulation of natural killer (NK) cell, B-cell, and T-cell proliferation, NK cell differentiation, and memory CD8 T cell responses. IL15 is associated with diseases and conditions including rheumatoid arthritis, psoriasis, and inflammatory bowel disease.

### Antibodies

RVT-3101, a human monoclonal antibody that binds TL1A, is in development for treatment of inflammatory bowel disease. AVTX-009, APG-777; AIO-001, and CALY-002 are human monoclonal antibodies that bind IL1β, IL13, TSLP, and IL15, respectively. These antibodies are in development for treatment of hidradenitis suppurativa (AVTX-009) atopic dermatitis and asthma (APG-777), asthma (AIO-001), and eosinophilic esophagitis and celiac disease (CALY-002), respectively. However, these antibodies have a number of limitations including pharmacokinetic properties that require administration every month. In some embodiments, limitations also include high immunogenicity observed in Phase 1 clinical studies. These limitations demonstrate the need for development of antibodies with improved pharmacokinetics, reduced immunogenicity, and equipotent or superior efficacy.

In contrast, the antibodies provided herein (e.g., anti-TL1A, anti-IL1β, anti-IL13, anti-TSLP, and anti-IL15 antibodies) demonstrate advantages as described herein that make them useful for treatment of autoimmune diseases and conditions.

Provided herein are antibodies and antigen-binding portions thereof that specifically bind a target antigen (e.g., TL1A IL1β, IL13, TSLP, or IL15). The disclosed antibodies, nucleic acids, vectors, and cells may be understood more readily by reference to the following detailed description taken in connection with the accompanying figures, which form a part of this disclosure. It is to be understood that the disclosed antibodies, nucleic acids, vectors, and cells are not limited to the specific antibodies, nucleic acids, vectors, and cells described and/or shown herein, and that the terminology used herein is for the purpose of describing particular embodiments by way of example only and is not intended to be limiting of the claimed antibodies, nucleic acids, vectors, and cells.

Unless specifically stated otherwise, any description as to a possible mechanism or mode of action or reason for improvement is meant to be illustrative only, and the disclosed antibodies, nucleic acids, vectors, and cells are not to be constrained by the correctness or incorrectness of any such suggested mechanism or mode of action or reason for improvement.

The antibodies provided herein have several advantages over their respective comparator antibodies. For example, the anti-TL1A antibodies provided herein have several advantages over RVT-3101 (disclosed, e.g., in U.S. Pat. No. 9,023,358 B2 and WO2015073580A1). The anti-TL1A antibodies provided herein have been engineered to have improved potency in blocking DR3 signaling induced by TL1A. The anti-IL13 antibodies provided herein have been engineered to have improved potency in blocking IL4/IL13 signaling pathway and target cell proliferation induced by IL13. The anti-IL1β antibodies provided herein have been engineered to have improved potency in blocking IL6 release and pro-inflammatory responses induced by IL1β. The anti-IL15 antibodies provided herein have been engineered to have improved potency in blocking pro-inflammatory responses induced by IL15 and lower self-association/aggregation propensity. The anti-TSLP antibodies provided herein have been engineered to have improved potency in blocking release of inflammatory cytokines and transmission of downstream inflammatory signals induced by TSLP.

Critically, these antibodies also have improved pharmacokinetic properties (e.g., longer half-life) compared to RVT-3101. The anti-IL1β antibodies provided herein have several advantages over AVTX-009 (disclosed, e.g., in WO2021150486A1). The anti-IL13 antibodies provided herein have several advantages over APG-777 (disclosed, e.g., in WO2023245187A2). The anti-TSLP antibodies provided herein have several advantages over AIO-001 (disclosed, e.g., in WO2020244544A1). The anti-IL15 antibodies provided herein have several advantages over CALY-002 (disclosed, e.g., in U.S. Ser. No. 11/130,806B2). The bispecific antibodies provided herein (e.g., the bispecific IL13×TSLP antibodies) have several advantages over the respective comparator antibodies for the first antigen-binding domain and the second antigen-binding domain (e.g., over APG-777 and AIO-001).

Antibodies and antigen-binding portions disclosed herein specifically bind a human target antigen (e.g., human TL1A). In some embodiments, the antibodies and antigen-binding portions disclosed herein bind human TL1A, IL1β, IL13, TSLP, or IL15. In some embodiments, antibodies and antigen-binding portions may cross-react with the target antigen (e.g., TL1A) from species other than human, for example, cynomolgus monkey (Macaca fascicularis) target antigen, rhesus monkey (Macaca mulatta) target antigen, and/or mouse (Mus musculus) target antigen. For example, the anti-TL1A antibodies and antigen-binding portions disclosed herein may cross-react with cynomolgus monkey (Macaca fascicularis) TL1A, rhesus monkey (Macaca mulatta) TL1A, and/or mouse (Mus musculus) TL1A. In some embodiments, an antibody may be specific for only the human target antigen (e.g., human TL1A) and may exhibit no non-human cross-reactivity. Exemplary amino acid sequences of target antigens (e.g., TL1A) are provided in Table 8.

The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable region (abbreviated herein as VH region) and a heavy chain constant region. The heavy chain constant region comprises three domains, CH1, CH2 and CH3. Each light chain comprises a light chain variable region (abbreviated herein as VL region) and a light chain constant region. The light chain constant region comprises one domain, CL. The VH and VL regions can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4. The CDR definition used in the present application is the Kabat definition unless otherwise stated (Kabat et al., Sequences of Proteins of Immunological Interest, 5^{th} ed. Bethesda, MD: Public Health Service, National Institutes of Health (1991)).

The term “Fe region” is used to define a C-terminal region of an immunoglobulin heavy chain. The “Fe region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU index as in Kabat. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins. Exemplary amino acid sequences of Fc regions are provided in Table 9.

Immunoglobulin molecules can be of any type (e.g., IgG, IgE, IgM, IgD, IgA or IgY) and class (e.g., IgG1, IgG2, IgG3, IgG4, IgAQ1 or IgA2) or subclass. IgG, IgD, and IgE antibodies generally contain two identical heavy chains and two identical light chains and two antigen combining domains, each composed of a VH) and a VL. Generally IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

The term “antigen-binding portion” or “antigen-binding fragment” of an antibody (or “antibody portion” or “antibody fragment”), as used herein, refers to one or more fragments of an antibody that retain the ability to specifically bind to an antigen (e.g., TL1A). It has been shown that the antigen-binding function of an antibody can be performed by portions or fragments of a full-length antibody. Examples of binding portions encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb (domain antibody) fragment (Ward et al., (1989) Nature 341:544-546; WO 90/05144 A1, each herein incorporated by reference in its entirety), which comprises a single variable domain; and (vi) an isolated complementarity determining region (CDR). The disclosure also encompasses a Fab′ fragment. Fab′ fragments can be formed by the reduction of F(ab′)₂ fragments. Fab′ is derived from F(ab′)₂; therefore, it may contain a small portion of Fc. Furthermore, although the two domains of the Fv fragment, VL and VH, are coded for by separate genes, they can be joined, using recombinant methods, by a synthetic linker that enables them to be made as a single protein chain in which the VL and VH domains pair to form monovalent molecules (known as single chain Fv (scFv). See e.g., Bird et al. (1988) Science 242:423-426; Huston et al. (1988) Proc. Natl. Acad. Sci. USA 85:5879-5883. Such single chain antibodies are also intended to be encompassed within the term “antigen-binding portion” of an antibody. In some embodiments, scFv molecules may be incorporated into a fusion protein. In some embodiments, provided herein is a single chain camelid antibody. In some embodiments, provided herein is a shark heavy chain antibody (V-NAR). See, English et al. (2020) Antibody Therapeutics, 3(1):1-9. Examples of antigen-binding portions are known in the art (Kontermann and Dubel eds., Antibody Engineering (2001) Springer-Verlag. New York. 790 pp.). In some embodiments, provided herein is a single domain antibody. In general, the term “antibody” when used herein encompasses an “antibody portion”. An antibody portion generally retains the antigen-binding properties of a full-length antibody.

Antibodies and antibody portions provided herein may be in multispecific (e.g., bispecific or trispecific) formats. Such multispecific molecules specifically bind to two or more different molecular targets or epitopes. In some embodiments, an antibody or an antigen-binding portion is a bispecific molecule that binds specifically to a first antigen and a second antigen, wherein the first antigen and the second antigen are not the same. In some embodiments, an antibody or an antigen-binding portion is a bispecific molecule that binds specifically to a first antigen and a second antigen, wherein the first antigen is IL13, and the second antigen is not IL13. In some embodiments, an antibody or an antigen-binding portion is a bispecific molecule that binds specifically to a first antigen and a second antigen, wherein the first antigen is TSLP, and the second antigen is not TSLP. In some embodiments, an antibody or an antigen-binding portion is a bispecific molecule that binds specifically to a first antigen and a second antigen, wherein the first antigen is IL13, and the second antigen is TSLP. In some embodiments, an antibody or an antigen-binding portion is a bispecific molecule that binds specifically to a first antigen and a second antigen, wherein the first antigen is TL1A and the second antigen is not TL1A. In some embodiments, an antibody or an antigen-binding portion is a diabody. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al. (1993) Proc. Natl. Acad. Sci. USA 90:6444-6448; Poljak et al. (1994) Structure 2:1121-1123). In some embodiments, an antibody or an antigen-binding portion is a triabody, a tetrabody, a bis-scFv or a tandem scFv. In some embodiments, an antibody or an antigen-binding portion is a dual affinity re-targeting protein.

In some embodiments, an antigen-binding portion disclosed herein is a Fab, a Fab′, a F(ab′)₂, a Fv, a scFv, a maxibody, a minibody, a diabody, a triabody, a tetrabody, or a bis-scFv.

As used herein, the terms “immunological binding” and “immunological binding properties” refer to the non-covalent interactions of the type which occur between an immunoglobulin molecule (e.g., antibody or antigen-binding portion thereof) and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity and is equal to the dissociation constant K_{d}. (See, Davies et al. (1990) Annual Rev Biochem 59:439-473). An antibody or antigen-binding portion provided herein is said to specifically bind the target antigen (e.g., TL1A) when the equilibrium binding constant (K_{d}) is ≤10 μM, preferably ≤10 nM, more preferably ≤10 nM, and most preferably ≤100 pM to about 1 pM, as measured by assays such as radioligand binding assays or similar assays known to those skilled in the art. One method for determining the K_{d} of an antibody is by using surface plasmon resonance (SPR), typically using a biosensor system such as a Biacore® system.

In some embodiments, an antibody or antigen-binding portion provided herein (e.g., an anti-TL1A antibody or antigen-binding portion) is monovalent or bivalent and comprises a single or double chain. Functionally, the binding affinity of an antibody or antigen-binding portion may be within the range of 10⁻⁵M to 10⁻¹² M. For example, the binding affinity of an antibody or antigen-binding portion is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 1010 M to 10⁻¹¹ M, from 10⁻⁵ M to 10⁻¹⁰ M, from 10⁻⁶ M to 1010 M, from 10⁻⁷ M to 10⁻¹⁰ M, from 10⁻⁸ M to 10⁻¹⁰ M, from 10⁻⁹ M to 10⁻¹⁰ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M or from 10⁻⁵ M to 10⁻⁶ M.

In some embodiments, an antibody or an antigen-binding portion thereof disclosed herein does not comprise one or more of the amino acid sequences comprised by a comparator antibody, as provided in Table 6. In some embodiments, an antibody or an antigen-binding portion thereof does not comprise: a) an HCDR1 of SEQ ID NO: 25, HCDR2 of SEQ ID NO: 30, HCDR3 of SEQ ID NO: 3, LCDR1 of SEQ ID NO: 4, LCDR2 of SEQ ID NO: 5, and LCDR3 of SEQ ID NO: 12; b) an HCDR1 of SEQ ID NO: 43, HCDR2 of SEQ ID NO: 44, HCDR3 of SEQ ID NO: 111, LCDR1 of SEQ ID NO: 46, LCDR2 of SEQ ID NO: 47, and LCDR3 of SEQ ID NO: 48; c) an HCDR1 of SEQ ID NO: 53, HCDR2 of SEQ ID NO: 54, HCDR3 of SEQ ID NO: 112, LCDR1 of SEQ ID NO: 56, LCDR2 of SEQ ID NO: 57, and LCDR3 of SEQ ID NO: 58; d) an HCDR1 of SEQ ID NO: 66, HCDR2 of SEQ ID NO: 114, HCDR3 of SEQ ID NO: 115, LCDR1 of SEQ ID NO: 69, LCDR2 of SEQ ID NO: 70, and LCDR3 of SEQ ID NO: 71; or e) an HCDR1 of SEQ ID NO: 76, HCDR2 of SEQ ID NO: 77, HCDR3 of SEQ ID NO: 113, LCDR1 of SEQ ID NO: 79, LCDR2 of SEQ ID NO: 80 and LCDR3 of SEQ ID NO: 81.

Table 6 provides the amino acid sequences of the RVT-3101 anti-TL1A antibody variable regions with highlighted CDRs as defined herein (“Kabat” scheme). Table 6 further provides the amino acid sequences of the AVTX-009 anti-IL10 antibody, the APG-777 anti-IL13 antibody, the CALY-002 anti-IL15 antibody, and the AIO-001 anti-TSLP antibody variable regions with highlighted CDRs as defined herein (“Kabat” scheme).

In some embodiments, an anti-TL1A antibody or an antigen-binding portion thereof does not comprise one or more of the RVT-3101 amino acid sequences provided in Table 6. In some embodiments, an anti-TL1A antibody or an antigen-binding portion thereof does not comprise an HCDR1 of SEQ ID NO: 25, HCDR2 of SEQ ID NO: 30, HCDR3 of SEQ ID NO: 3, LCDR1 of SEQ ID NO: 4, LCDR2 of SEQ ID NO: 5 and LCDR3 of SEQ ID NO: 12.

In some embodiments, an anti-IL1P antibody or an antigen-binding portion thereof does not comprise one or more of the AVTX-009 amino acid sequences provided in Table 6. In some embodiments, an anti-IL1P antibody or an antigen-binding portion thereof does not comprise an HCDR1 of SEQ ID NO: 43, HCDR2 of SEQ ID NO: 44, HCDR3 of SEQ ID NO: 111, LCDR1 of SEQ ID NO: 46, LCDR2 of SEQ ID NO: 47, and LCDR3 of SEQ ID NO: 48.

In some embodiments, an anti-IL13 antibody or an antigen-binding portion thereof does not comprise one or more of the APG-777 amino acid sequences provided in Table 6. In some embodiments, an anti-IL13 antibody or an antigen-binding portion thereof does not comprise an HCDR1 of SEQ ID NO: 53, HCDR2 of SEQ ID NO: 54, HCDR3 of SEQ ID NO: 112, LCDR1 of SEQ ID NO: 56, LCDR2 of SEQ ID NO: 57, and LCDR3 of SEQ ID NO: 58.

In some embodiments, an anti-IL15 antibody or an antigen-binding portion thereof does not comprise one or more of the CALY-002 amino acid sequences provided in Table 6. In some embodiments, an anti-IL15 antibody or an antigen-binding portion thereof does not comprise an HCDR1 of SEQ ID NO: 66, HCDR2 of SEQ ID NO: 114, HCDR3 of SEQ ID NO: 115, LCDR1 of SEQ ID NO: 69, LCDR2 of SEQ ID NO: 70, and LCDR3 of SEQ ID NO: 71.

In some embodiments, an anti-TSLP antibody or an antigen-binding portion thereof does not comprise one or more of the AIO-001amino acid sequences provided in Table 6. In some embodiments, an anti-TSLP antibody or an antigen-binding portion thereof does not comprise an HCDR1 of SEQ ID NO: 76, HCDR2 of SEQ ID NO: 77, HCDR3 of SEQ ID NO: 113, LCDR1 of SEQ ID NO: 79, LCDR2 of SEQ ID NO: 80 and LCDR3 of SEQ ID NO: 81. In some embodiments, antibodies disclosed herein are antagonist antibodies for a target antigen (e.g. TL1A). As used herein, an “antagonist” or an “antagonist antibody” (interchangeably termed “anti-[target antigen] antibody”, wherein [target antigen] is replaced with any target antigen disclosed herein) refers to an antibody which is able to bind to the target antigen and inhibit biological activity and/or downstream pathway(s) mediated by the target antigen. An antagonist antibody encompasses antibodies that can block, antagonize, suppress or reduce (including significantly) the biological activity of the target antigen, including downstream pathways mediated by target antigen signalling, such as receptor binding and/or elicitation of a cellular response to the target antigen. For the purposes of the present disclosure, it will be explicitly understood that the term “antagonist antibody” encompasses all the terms, titles, and functional states and characteristics whereby the target antigen itself, and the biological activity of the target antigen (including but not limited to its ability to suppress the activation of anti-tumor cell activity of T cells), or the consequences of the activity or biological activity, are substantially nullified, decreased, or neutralized in a meaningful degree.

For example, an “anti-TL1A antagonist antibody” or “anti-TL1A antibody” refers to an antibody which is able to bind to TL1A and inhibit TL1A biological activity and/or downstream pathway(s) mediated by TL1A signalling. An anti-TL1A antagonist antibody encompasses antibodies that can block, antagonize, suppress or reduce (including significantly) TL1A biological activity, including downstream pathways mediated by TL1A signalling, such as receptor binding and/or elicitation of a cellular response to TL1A. For the purposes of the present disclosure, it will be explicitly understood that the term “anti-TL1A antagonist antibody” encompasses all the terms, titles, and functional states and characteristics whereby TL1A itself, and TL1A biological activity (including but not limited to its ability to suppress the activation of anti-tumor cell activity of T cells), or the consequences of the activity or biological activity, are substantially nullified, decreased, or neutralized in a meaningful degree.

Further provided herein are antibodies or antigen-binding fragments thereof that bind TL1A, wherein the antibody or antigen-binding fragment thereof comprises a heavy chain variable region (VH) and a light chain variable region (VL). In some embodiments, the antibody or antigen-binding fragment thereof comprises a VH with at least 70%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 7, 13, 23, and 28. In some embodiments, the antibody or antigen-binding fragment thereof comprises a VL with at least 70%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 8, 14, 18, and 20. In some embodiments, the antibody or antigen-binding fragment thereof comprises: a) a VH with at least 70%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 7, 13, 23, and 28; and b) a VL with at least 70%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 8, 14, 18, and 20. In some embodiments, the antibody or antigen-binding fragment thereof comprises: a) a VH with at least 90% sequence identity to any one of SEQ ID NOs: 7, 13, 23, and 28; and b) a VL with at least 90% sequence identity to any one of SEQ ID NOs: 8, 14, 18, and 20.

In some embodiments, the antibody or antigen-binding fragment thereof comprises:
a) a heavy chain variable region (VH) comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 7; and a light chain variable region (VL) with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 8;
b) a VH comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 13; and a VL comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 14;
c) a VH comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 7 and a VL comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 18;
d) a VH comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 7 and a VL comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 20;
e) a VH comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 23 and a VL comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 20; or
f) a VH comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 28 and a VL comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 20.

Further provided herein is an anti-TL1A antibody or an antigen-binding portion thereof that comprises a combination of a VH region amino acid sequence and VL region amino acid sequence, and/or a combination of CDR amino acid sequences provided in Table1. In some embodiments, the antibody or antigen-binding fragment thereof comprises a VH comprising the amino acid sequence of any one of SEQ ID NOs: 7, 13, 23, and 28. In some embodiments, the antibody or antigen-binding fragment thereof comprises a VL comprising the amino acid sequence of any one of SEQ ID NOs: 8, 14, 18, and 20. In some embodiments, the antibody or antigen-binding fragment thereof comprises: a) a VH comprising the amino acid sequence of any one of SEQ ID NOs: 7, 13, 23, and 28; and b) a VL comprising the amino acid sequence of any one of SEQ ID NOs: 8, 14, 18, and 20. In some embodiments, the VH region amino acid sequence and/or the VL region amino acid sequence comprises a signal sequence (also known as a signal peptide) at the amino-terminus.

Disclosed herein are antibodies or antigen-binding fragments thereof that bind tumor necrosis factor-like ligand 1A (TL1A), wherein the antibodies or antigen-binding fragments thereof comprise:
a) a heavy chain complementarity determining region 1 (HCDR1) having at least 90% sequence identity with SEQ ID NO: 1, a HCDR2 having at least 90% sequence identity with SEQ ID NO: 2, a HCDR3 having at least 90% sequence identity with SEQ ID NO: 3, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6;
b) a HCDR1 having at least 90% sequence identity with SEQ ID NO: 1, a HCDR2 having at least 90% sequence identity with SEQ ID NO: 11, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 5, and a LCDR3 having at least 90% sequence identity with SEQ ID NO: 12;
c) a HCDR1 having at least 90% sequence identity with SEQ ID NO: 1, a HCDR2 having at least 90% sequence identity with SEQ ID NO: 2, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 6;
d) a HCDR1 having at least 90% sequence identity with SEQ ID NO: 1, a HCDR2 having at least 90% sequence identity with SEQ ID NO: 2, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 12;
e) a HCDR1 having at least 90% sequence identity with SEQ ID NO: 1, a HCDR2 having at least 90% sequence identity with SEQ ID NO: 22, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 12;
f) a HCDR1 having at least 90% sequence identity with SEQ ID NO: 25, a HCDR2 having at least 90% sequence identity with SEQ ID NO: 2, a HCDR3 having at least 90% sequence identity with SEQ ID NO: 3, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6; or
g) a HCDR1 having at least 90% sequence identity with SEQ ID NO: 25, a HCDR2 having at least 90% sequence identity with SEQ ID NO: 2, a HCDR3 having at least 90% sequence identity with SEQ ID NO: 27, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6;
   wherein the numbering of HCDR1 is according to the AbM numbering scheme (as described in e.g. Abhinandan et al. Molecular immunology 45, no. 14 (2008): 3832-3839).

In some embodiments, the antibodies or antigen-binding fragments thereof comprise:
a) a heavy chain complementarity determining region 1 (HCDR1) comprising the amino acid sequence of SEQ ID NO: 1, a HCDR2 comprising the amino acid sequence of SEQ ID NO: 2, a HCDR3 comprising the amino acid sequence of SEQ ID NO: 3, a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
b) a HCDR1 comprising the amino acid sequence of SEQ ID NO: 1, a HCDR2 comprising the amino acid sequence of SEQ ID NO: 11, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 12;
c) a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 6;
d) a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 12;
e) a HCDR1 comprising the amino acid sequence of SEQ ID NO: 1, a HCDR2 comprising the amino acid sequence of SEQ ID NO: 22, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 12;
f) a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6; or
g) a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 27, a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
wherein the numbering of HCDR1 is according to the AbM numbering scheme (as described in e.g. Abhinandan et al. Molecular immunology 45, no. 14 (2008): 3832-3839).

Disclosed herein are antibodies or antigen-binding fragments thereof that bind tumor necrosis factor-like ligand 1A (TL1A), wherein the antibodies or antigen-binding fragments thereof comprise:
a) a heavy chain complementarity determining region 1 (HCDR1) comprising the amino acid sequence of SEQ ID NO: 25, a HCDR2 comprising the amino acid sequence of SEQ ID NO: 2, a HCDR3 comprising the amino acid sequence of SEQ ID NO: 3, a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
b) a HCDR1 comprising the amino acid sequence of SEQ ID NO: 25, a HCDR2 comprising the amino acid sequence of SEQ ID NO: 11, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 12;
c) a HCDR1 comprising the amino acid sequence of SEQ ID NO: 25, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 6;
d) a HCDR1 comprising the amino acid sequence of SEQ ID NO: 25, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 12;
e) a HCDR1 comprising the amino acid sequence of SEQ ID NO: 25, a HCDR2 comprising the amino acid sequence of SEQ ID NO: 22, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 12;
f) a HCDR1 comprising the amino acid sequence of SEQ ID NO: 25, a HCDR2 comprising the amino acid sequence of SEQ ID NO: 2, a HCDR3 comprising the amino acid sequence of SEQ ID NO: 3, a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6; or
g) a HCDR1 comprising the amino acid sequence of SEQ ID NO: 25, a HCDR2 comprising the amino acid sequence of SEQ ID NO: 2, a HCDR3 comprising the amino acid sequence of SEQ ID NO: 27, a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
wherein the numbering of HCDR1 is according to the EU index as in Kabat.

In some embodiments, the antibodies or antigen-binding fragments thereof comprise:
a) a heavy chain complementarity determining region 1 (HCDR1) having at least 90% sequence identity with SEQ ID NO: 25, a HCDR2 having at least 90% sequence identity with SEQ ID NO: 2, a HCDR3 having at least 90% sequence identity with SEQ ID NO: 3, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6;
b) a HCDR1 having at least 90% sequence identity with SEQ ID NO: 25, a HCDR2 having at least 90% sequence identity with SEQ ID NO: 11, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 5, and a LCDR3 having at least 90% sequence identity with SEQ ID NO: 12;
c) a HCDR1 having at least 90% sequence identity with SEQ ID NO: 25, a HCDR2 having at least 90% sequence identity with SEQ ID NO: 2, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 6;
d) a HCDR1 having at least 90% sequence identity with SEQ ID NO: 25, a HCDR2 having at least 90% sequence identity with SEQ ID NO: 2, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 12;
e) a HCDR1 having at least 90% sequence identity with SEQ ID NO: 25, a HCDR2 having at least 90% sequence identity with SEQ ID NO: 22, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 12;
f) a HCDR1 having at least 90% sequence identity with SEQ ID NO: 25, a HCDR2 having at least 90% sequence identity with SEQ ID NO: 2, a HCDR3 having at least 90% sequence identity with SEQ ID NO: 3, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6; or
g) a HCDR1 having at least 90% sequence identity with SEQ ID NO: 25, a HCDR2 having at least 90% sequence identity with SEQ ID NO: 2, a HCDR3 having at least 90% sequence identity with SEQ ID NO: 27, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6;
wherein the numbering of HCDR1 is according to the EU index as in Kabat.

The antibodies or antigen-binding fragments thereof can comprise a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6.

The antibodies or antigen-binding fragments thereof can comprise a HCDR1 comprising the amino acid sequence of SEQ ID NO: 1, a HCDR2 comprising the amino acid sequence of SEQ ID NO: 11, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 12.

The antibodies or antigen-binding fragments thereof can comprise a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 6.

The antibodies or antigen-binding fragments thereof can comprise a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 12.

The antibodies or antigen-binding fragments thereof can comprise a HCDR1 comprising the amino acid sequence of SEQ ID NO: 1, a HCDR2 comprising the amino acid sequence of SEQ ID NO: 22, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 12.

The antibodies or antigen-binding fragments thereof can comprise a HCDR1 comprising the amino acid sequence of SEQ ID NO: 25, a HCDR2 comprising the amino acid sequence of SEQ ID NO: 2, a HCDR3 comprising the amino acid sequence of SEQ ID NO: 3, a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6.

The antibodies or antigen-binding fragments thereof can comprise a HCDR1 comprising the amino acid sequence of SEQ ID NO: 25, a HCDR2 comprising the amino acid sequence of SEQ ID NO: 2, a HCDR3 comprising the amino acid sequence of SEQ ID NO: 27, a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6.

Provided herein is an anti-TL1A antibody or an antigen-binding portion thereof, wherein the antibody or antigen-binding portion comprises a VH region and a VL region. In some embodiments, the VH region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 7. In some embodiments, the VH region comprises the amino acid sequence of SEQ ID NO: 7. In some embodiments, the VH region consists of the amino acid sequence of SEQ ID NO: 7. In some embodiments, the VH region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 13. In some embodiments, the VH region comprises the amino acid sequence of SEQ ID NO: 13. In some embodiments, the VH region consists of the amino acid sequence of SEQ ID NO: 13. In some embodiments, the VH region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 23. In some embodiments, the VH region comprises the amino acid sequence of SEQ ID NO: 23. In some embodiments, the VH region consists of the amino acid sequence of SEQ ID NO: 23. In some embodiments, the VH region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 28. In some embodiments, the VH region comprises the amino acid sequence of SEQ ID NO: 28. In some embodiments, the VH region consists of the amino acid sequence of SEQ ID NO: 28.

In some embodiments, the VL region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 8. In some embodiments, the VL region comprises the amino acid sequence of SEQ ID NO: 8. In some embodiments, the VL region consists of the amino acid sequence of SEQ ID NO: 8. In some embodiments, the VL region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 14. In some embodiments, the VL region comprises the amino acid sequence of SEQ ID NO: 14. In some embodiments, the VL region consists of the amino acid sequence of SEQ ID NO: 14. In some embodiments, the VL region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 18. In some embodiments, the VL region comprises the amino acid sequence of SEQ ID NO: 18. In some embodiments, the VL region consists of the amino acid sequence of SEQ ID NO: 18. In some embodiments, the VL region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 20. In some embodiments, the VL region comprises the amino acid sequence of SEQ ID NO: 20. In some embodiments, the VL region consists of the amino acid sequence of SEQ ID NO: 20.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 7 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 8.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 13 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 14.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 7 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 18.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 7 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 20.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 23 and the VL region amino acid sequence has at least 90%, %, at least 91%, at least 92%, at least 93%, at least 94%, at least 95%, at least 96%, at least 97%, at least 98%, or at least 99% sequence identity to the amino acid sequence of SEQ ID NO: 20.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 28 and the VL region amino acid sequence has at least 90%, %, at least 91%, at least 92%, at least 93%, at least 94%, at least 95%, at least 96%, at least 97%, at least 98%, or at least 99% sequence identity to the amino acid sequence of SEQ ID NO: 20.

The antibodies or antigen-binding fragments thereof can comprise:
a) a heavy chain variable region (VH) comprising the amino acid sequence of SEQ ID NO: 7 and a light chain variable region (VL) comprising the amino acid sequence of SEQ ID NO: 8;
b) a VH comprising the amino acid sequence of SEQ ID NO: 13 and a VL comprising the amino acid sequence of SEQ ID NO: 14;
c) a VH comprising the amino acid sequence of SEQ ID NO: 7 and a VL comprising the amino acid sequence of SEQ ID NO: 18;
d) a VH comprising the amino acid sequence of SEQ ID NO: 7 and a VL comprising the amino acid sequence of SEQ ID NO: 20;
e) a VH comprising the amino acid sequence of SEQ ID NO: 23 and a VL comprising the amino acid sequence of SEQ ID NO: 20; or
f) a VH comprising the amino acid sequence of SEQ ID NO: 28 and a VL comprising the amino acid sequence of SEQ ID NO: 20.

The antibodies or antigen-binding fragments thereof can comprise a VH comprising the amino acid sequence of SEQ ID NO: 7 and a VL comprising the amino acid sequence of SEQ ID NO: 8.

The antibodies or antigen-binding fragments thereof can comprise a VH comprising the amino acid sequence of SEQ ID NO: 13 and a VL comprising the amino acid sequence of SEQ ID NO: 14.

The antibodies or antigen-binding fragments thereof can comprise a VH comprising the amino acid sequence of SEQ ID NO: 7 and a VL comprising the amino acid sequence of SEQ ID NO: 18.

The antibodies or antigen-binding fragments thereof can comprise a VH comprising the amino acid sequence of SEQ ID NO: 7 and a VL comprising the amino acid sequence of SEQ ID NO: 20.

The antibodies or antigen-binding fragments thereof can comprise a VH comprising the amino acid sequence of SEQ ID NO: 23 and a VL comprising the amino acid sequence of SEQ ID NO: 20.

The antibodies or antigen-binding fragments thereof can comprise a VH comprising the amino acid sequence of SEQ ID NO: 28 and a VL comprising the amino acid sequence of SEQ ID NO: 20.

Provided herein is an anti-TL1A antibody or an antigen-binding portion thereof, wherein the antibody or antigen-binding portion comprises a VH region and a VL region, wherein (a) the VH region amino acid sequence comprises SEQ ID NO: 7; and the VL region amino acid sequence comprises SEQ ID NO: 8, with 1, 2 or 3 conservative amino acid substitutions in the VH region sequence, the VL region sequence, or both the VH region and the VL region sequences; (b) the VH region amino acid sequence comprises SEQ ID NO: 13; and the VL region amino acid sequence comprises SEQ ID NO: 14, with 1, 2 or 3 conservative amino acid substitutions in the VH region sequence, the VL region sequence, or both the VH region and the VL region sequences; (c) the VH region amino acid sequence comprises SEQ ID NO: 7; and the VL region amino acid sequence comprises SEQ ID NO: 18, with 1, 2 or 3 conservative amino acid substitutions in the VH region sequence, the VL region sequence, or both the VH region and the VL region sequences; (d) the VH region amino acid sequence comprises SEQ ID NO: 7; and the VL region amino acid sequence comprises SEQ ID NO: 20, with 1, 2 or 3 conservative amino acid substitutions in the VH region sequence, the VL region sequence, or both the VH region and the VL region sequences; (e) the VH region amino acid sequence comprises SEQ ID NO: 23; and the VL region amino acid sequence comprises SEQ ID NO: 20, with 1, 2 or 3 conservative amino acid substitutions in the VH region sequence, the VL region sequence, or both the VH region and the VL region sequences; (f) the VH region amino acid sequence comprises SEQ ID NO: 28; and the VL region amino acid sequence comprises SEQ ID NO: 20, with 1, 2 or 3 conservative amino acid substitutions in the VH region sequence, the VL region sequence, or both the VH region and the VL region sequences. In some embodiments, conservative amino acid substitutions are made only in the FR sequences and not in the CDR sequences of an antibody or antigen-binding portion.

Further provided herein are antibodies or antigen-binding fragments thereof that bind IL1β, wherein the antibody or antigen-binding fragment thereof comprises a heavy chain variable region (VH) and a light chain variable region (VL). In some embodiments, the antibody or antigen-binding fragment thereof comprises a VH with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 49. In some embodiments, the antibody or antigen-binding fragment thereof comprises a VL with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 50. In some embodiments, the antibody or antigen-binding fragment thereof comprises: a) a VH with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 49; and b) a VL with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 50. In some embodiments, the antibody or antigen-binding fragment thereof comprises: a) a VH with at least 90% sequence identity to SEQ ID NO: 49; and b) a VL with at least 90% sequence identity to SEQ ID NO: 50.

In some embodiments, the antibody or antigen-binding fragment thereof comprises: a heavy chain variable region (VH) comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 49; and a light chain variable region (VL) with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 50.

Further provided herein is an anti-IL1β antibody or an antigen-binding portion thereof that comprises a combination of a VH region amino acid sequence and VL region amino acid sequence, and/or a combination of CDR amino acid sequences provided in Table 2. In some embodiments, the antibody or antigen-binding fragment thereof comprises a VH comprising the amino acid sequence of SEQ ID NO: 49. In some embodiments, the antibody or antigen-binding fragment thereof comprises a VL comprising the amino acid sequence of SEQ ID NO: 50. In some embodiments, the antibody or antigen-binding fragment thereof comprises: a) a VH comprising the amino acid sequence of any one of SEQ ID NO: 49; and b) a VL comprising the amino acid sequence of any one of SEQ ID NO: 50. In some embodiments, the VH region amino acid sequence and/or the VL region amino acid sequence comprises a signal sequence (also known as a signal peptide) at the amino-terminus.

Disclosed herein are antibodies or antigen-binding fragments thereof that bind interleukin-1β (IL1β), wherein the antibodies or antigen-binding fragments thereof comprise: a heavy chain complementarity determining region 1 (HCDR1) comprising the amino acid sequence of SEQ ID NO: 43, a HCDR2 comprising the amino acid sequence of SEQ ID NO: 44, a HCDR3 comprising the amino acid sequence of SEQ ID NO: 45, a light chain complementarity determining region 1 (LCDR1) comprising the amino acid sequence of SEQ ID NO: 46, a LCDR2 comprising the amino acid sequence of SEQ ID NO: 47, and a LCDR3 comprising the amino acid sequence of SEQ ID NO: 48; wherein the numbering of HCDR1 is according to the EU index as in Kabat.

In some embodiments, the antibodies or antigen-binding fragments thereof comprise: a heavy chain complementarity determining region 1 (HCDR1) having at least 90% sequence identity with SEQ ID NO: 43, a HCDR2 having at least 90% sequence identity with SEQ ID NO: 44, a HCDR3 having at least 90% sequence identity with SEQ ID NO: 45, a light chain complementarity determining region 1 (LCDR1) having at least 90% sequence identity with SEQ ID NO: 46, a LCDR2 having at least 90% sequence identity with SEQ ID NO: 47, and a LCDR3 having at least 90% sequence identity with SEQ ID NO: 48; wherein the numbering of HCDR1 is according to the EU index as in Kabat.

The antibodies or antigen-binding fragments thereof can comprise a HCDR1 comprising the amino acid sequence of SEQ ID NO: 43, a HCDR2 comprising the amino acid sequence of SEQ ID NO: 44, a HCDR3 comprising the amino acid sequence of SEQ ID NO: 45, a LCDR1 comprising the amino acid sequence of SEQ ID NO: 46, a LCDR2 comprising the amino acid sequence of SEQ ID NO: 47, and a LCDR3 comprising the amino acid sequence of SEQ ID NO: 48.

Provided herein is an anti-IL1β antibody or an antigen-binding portion thereof, wherein the antibody or antigen-binding portion comprises a VH region and a VL region. In some embodiments, the VH region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 49. In some embodiments, the VH region comprises the amino acid sequence of SEQ ID NO: 49. In some embodiments, the VH region consists of the amino acid sequence of SEQ ID NO: 49.

In some embodiments, the VL region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 50. In some embodiments, the VL region comprises the amino acid sequence of SEQ ID NO: 50. In some embodiments, the VL region consists of the amino acid sequence of SEQ ID NO: 50.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 49 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 50.

The antibodies or antigen-binding fragments thereof can comprise a heavy chain variable region (VH) comprising the amino acid sequence of SEQ ID NO: 49 and a light chain variable region (VL) comprising the amino acid sequence of SEQ ID NO: 50.

Provided herein is an anti-IL1β antibody or an antigen-binding portion thereof, wherein the antibody or antigen-binding portion comprises a VH region and a VL region, wherein (a) the VH region amino acid sequence comprises SEQ ID NO: 49; and the VL region amino acid sequence comprises SEQ ID NO: 50, with 1, 2 or 3 conservative amino acid substitutions in the VH region sequence, the VL region sequence, or both the VH region and the VL region sequences. In some embodiments, conservative amino acid substitutions are made only in the FR sequences and not in the CDR sequences of an antibody or antigen-binding portion.

Further provided herein are antibodies or antigen-binding fragments thereof that bind IL13, wherein the antibody or antigen-binding fragment thereof comprises a heavy chain variable region (VH) and a light chain variable region (VL). In some embodiments, the antibody or antigen-binding fragment thereof comprises a VH with at least 70%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 59 and 86. In some embodiments, the antibody or antigen-binding fragment thereof comprises a VL with at least 70%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 60 and 87. In some embodiments, the antibody or antigen-binding fragment thereof comprises: a) a VH with at least 70%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 59 and 86; and b) a VL with at least 70%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 60 and 87. In some embodiments, the antibody or antigen-binding fragment thereof comprises: a) a VH with at least 90% sequence identity to any one of SEQ ID NOs: 59 and 86; and b) a VL with at least 90% sequence identity to any one of SEQ ID NOs: 60 and 87.

In some embodiments, the antibody or antigen-binding fragment thereof comprises:
a. a heavy chain variable region (VH) comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 59; and a light chain variable region (VL) with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 60; or
b. a VH comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 86; and a VL comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 87.

Further provided herein is an anti-IL13 antibody or an antigen-binding portion thereof that comprises a combination of a VH region amino acid sequence and VL region amino acid sequence, and/or a combination of CDR amino acid sequences provided in Table 3. In some embodiments, the antibody or antigen-binding fragment thereof comprises a VH comprising the amino acid sequence of any one of SEQ ID NOs: 59 and 86. In some embodiments, the antibody or antigen-binding fragment thereof comprises a VL comprising the amino acid sequence of any one of SEQ ID NOs: 60 and 87. In some embodiments, the antibody or antigen-binding fragment thereof comprises: a) a VH comprising the amino acid sequence of any one of SEQ ID NOs: 59 and 86; and b) a VL comprising the amino acid sequence of any one of SEQ ID NOs: 60 and 87. In some embodiments, the VH region amino acid sequence and/or the VL region amino acid sequence comprises a signal sequence (also known as a signal peptide) at the amino-terminus.

Disclosed herein are antibodies or antigen-binding fragments thereof that bind interleukin-13 (IL13), wherein the antibodies or antigen-binding fragments thereof comprise:
a. a heavy chain complementarity determining region 1 (HCDR1) comprising the amino acid sequence of SEQ ID NO: 53, a HCDR2 comprising the amino acid sequence of SEQ ID NO: 54, a HCDR3 comprising the amino acid sequence of SEQ ID NO: 55, a light chain complementarity determining region 1 (LCDR1) comprising the amino acid sequence of SEQ ID NO: 56, a LCDR2 comprising the amino acid sequence of SEQ ID NO: 57, and a LCDR3 comprising the amino acid sequence of SEQ ID NO: 58; or
b. a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 82, a LCDR1 comprising the amino acid sequence of SEQ ID NO: 56, a LCDR2 comprising the amino acid sequence of SEQ ID NO: 57, and a LCDR3 comprising the amino acid sequence of SEQ ID NO: 58;
   wherein the numbering of HCDR1 is according to the EU index as in Kabat.

In some embodiments, the antibodies or antigen-binding fragments thereof comprise:
a. a heavy chain complementarity determining region 1 (HCDR1) having at least 90% sequence identity with SEQ ID NO: 53, a HCDR2 having at least 90% sequence identity with SEQ ID NO: 54, a HCDR3 having at least 90% sequence identity with SEQ ID NO: 55, a light chain complementarity determining region 1 (LCDR1) having at least 90% sequence identity with SEQ ID NO: 56, a LCDR2 having at least 90% sequence identity with SEQ ID NO: 57, and a LCDR3 having at least 90% sequence identity with SEQ ID NO: 58; or
b. a HCDR1 having at least 90% sequence identity with SEQ ID NO: 53, a HCDR2 having at least 90% sequence identity with SEQ ID NO: 54, a HCDR3 having at least 90% sequence identity with SEQ ID NO: 82, a LCDR1 having at least 90% sequence identity with SEQ ID NO: 56, a LCDR2 having at least 90% sequence identity with SEQ ID NO: 57, and a LCDR3 having at least 90% sequence identity with SEQ ID NO: 58;
   wherein the numbering of HCDR1 is according to the EU index as in Kabat.

The antibodies or antigen-binding fragments thereof can comprise a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 55, a LCDR1 comprising the amino acid sequence of SEQ ID NO: 56, a LCDR2 comprising the amino acid sequence of SEQ ID NO: 57, and a LCDR3 comprising the amino acid sequence of SEQ ID NO: 58.

The antibodies or antigen-binding fragments thereof can comprise a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 82, a LCDR1 comprising the amino acid sequence of SEQ ID NO: 56, a LCDR2 comprising the amino acid sequence of SEQ ID NO: 57, and a LCDR3 comprising the amino acid sequence of SEQ ID NO: 58.

Provided herein is an anti-IL13 antibody or an antigen-binding portion thereof, wherein the antibody or antigen-binding portion comprises a VH region and a VL region. In some embodiments, the VH region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 59. In some embodiments, the VH region comprises the amino acid sequence of SEQ ID NO: 59. In some embodiments, the VH region consists of the amino acid sequence of SEQ ID NO: 59. In some embodiments, the VH region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 86. In some embodiments, the VH region comprises the amino acid sequence of SEQ ID NO: 86. In some embodiments, the VH region consists of the amino acid sequence of SEQ ID NO: 86.

In some embodiments, the VL region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 60. In some embodiments, the VL region comprises the amino acid sequence of SEQ ID NO: 60. In some embodiments, the VL region consists of the amino acid sequence of SEQ ID NO: 60. In some embodiments, the VL region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 87. In some embodiments, the VL region comprises the amino acid sequence of SEQ ID NO: 87. In some embodiments, the VL region consists of the amino acid sequence of SEQ ID NO: 87.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 59 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 60.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 86 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 87.

The antibodies or antigen-binding fragments thereof can comprise:
a. a heavy chain variable region (VH) comprising the amino acid sequence of SEQ ID NO: 59 and a light chain variable region (VL) comprising the amino acid sequence of SEQ ID NO: 60; or
b. a VH comprising the amino acid sequence of SEQ ID NO: 86 and a VL comprising the amino acid sequence of SEQ ID NO: 87.

The antibodies or antigen-binding fragments thereof can comprise a VH comprising the amino acid sequence of SEQ ID NO: 59 and a VL comprising the amino acid sequence of SEQ ID NO: 60.

The antibodies or antigen-binding fragments thereof can comprise a VH comprising the amino acid sequence of SEQ ID NO: 86 and a VL comprising the amino acid sequence of SEQ ID NO: 87.

Provided herein is an anti-IL13 antibody or an antigen-binding portion thereof, wherein the antibody or antigen-binding portion comprises a VH region and a VL region, wherein (a) the VH region amino acid sequence comprises SEQ ID NO: 59; and the VL region amino acid sequence comprises SEQ ID NO: 60, with 1, 2 or 3 conservative amino acid substitutions in the VH region sequence, the VL region sequence, or both the VH region and the VL region sequences; or (b) the VH region amino acid sequence comprises SEQ ID NO: 86; and the VL region amino acid sequence comprises SEQ ID NO: 87, with 1, 2 or 3 conservative amino acid substitutions in the VH region sequence, the VL region sequence, or both the VH region and the VL region sequences. In some embodiments, conservative amino acid substitutions are made only in the FR sequences and not in the CDR sequences of an antibody or antigen-binding portion.

Further provided herein are antibodies or antigen-binding fragments thereof that bind IL15, wherein the antibody or antigen-binding fragment thereof comprises a heavy chain variable region (VH) and a light chain variable region (VL). In some embodiments, the antibody or antigen-binding fragment thereof comprises a VH with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 72. In some embodiments, the antibody or antigen-binding fragment thereof comprises a VL with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 73. In some embodiments, the antibody or antigen-binding fragment thereof comprises: a) a VH with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 72; and b) a VL with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 73. In some embodiments, the antibody or antigen-binding fragment thereof comprises: a) a VH with at least 90% sequence identity to SEQ ID NO: 72; and b) a VL with at least 90% sequence identity to SEQ ID NO: 73.

In some embodiments, the antibody or antigen-binding fragment thereof comprises: a heavy chain variable region (VH) comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 72; and a light chain variable region (VL) with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 73.

Further provided herein is an anti-IL15 antibody or an antigen-binding portion thereof that comprises a combination of a VH region amino acid sequence and VL region amino acid sequence, and/or a combination of CDR amino acid sequences provided in Table 4. In some embodiments, the antibody or antigen-binding fragment thereof comprises a VH comprising the amino acid sequence of SEQ ID NO: 72. In some embodiments, the antibody or antigen-binding fragment thereof comprises a VL comprising the amino acid sequence of SEQ ID NO: 73. In some embodiments, the antibody or antigen-binding fragment thereof comprises: a) a VH comprising the amino acid sequence of SEQ ID NO: 72; and b) a VL comprising the amino acid sequence of SEQ ID NO: 73. In some embodiments, the VH region amino acid sequence and/or the VL region amino acid sequence comprises a signal sequence (also known as a signal peptide) at the amino-terminus.

Disclosed herein are antibodies or antigen-binding fragments thereof that bind interleukin-15 (IL15), wherein the antibodies or antigen-binding fragments thereof comprise: a heavy chain complementarity determining region 1 (HCDR1) comprising the amino acid sequence of SEQ ID NO: 66, a HCDR2 comprising the amino acid sequence of SEQ ID NO: 67, a HCDR3 comprising the amino acid sequence of SEQ ID NO: 68, a light chain complementarity determining region 1 (LCDR1) comprising the amino acid sequence of SEQ ID NO: 69, a LCDR2 comprising the amino acid sequence of SEQ ID NO: 70, and a LCDR3 comprising the amino acid sequence of SEQ ID NO: 71; wherein the numbering of HCDR1 is according to the EU index as in Kabat.

In some embodiments, the antibodies or antigen-binding fragments thereof comprise: a heavy chain complementarity determining region 1 (HCDR1) having at least 90% sequence identity with SEQ ID NO: 66, a HCDR2 having at least 90% sequence identity with SEQ ID NO: 67, a HCDR3 having at least 90% sequence identity with SEQ ID NO: 68, a light chain complementarity determining region 1 (LCDR1) having at least 90% sequence identity with SEQ ID NO: 69, a LCDR2 having at least 90% sequence identity with SEQ ID NO: 70, and a LCDR3 having at least 90% sequence identity with SEQ ID NO: 71; wherein the numbering of HCDR1 is according to the EU index as in Kabat.

The antibodies or antigen-binding fragments thereof can comprise a HCDR1 comprising the amino acid sequence of SEQ ID NO: 66, a HCDR2 comprising the amino acid sequence of SEQ ID NO: 67, a HCDR3 comprising the amino acid sequence of SEQ ID NO: 68, a LCDR1 comprising the amino acid sequence of SEQ ID NO: 69, a LCDR2 comprising the amino acid sequence of SEQ ID NO: 70, and a LCDR3 comprising the amino acid sequence of SEQ ID NO: 71.

Provided herein is an anti-IL15 antibody or an antigen-binding portion thereof, wherein the antibody or antigen-binding portion comprises a VH region and a VL region. In some embodiments, the VH region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 72. In some embodiments, the VH region comprises the amino acid sequence of SEQ ID NO: 72. In some embodiments, the VH region consists of the amino acid sequence of SEQ ID NO: 72.

In some embodiments, the VL region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 73. In some embodiments, the VL region comprises the amino acid sequence of SEQ ID NO: 73. In some embodiments, the VL region consists of the amino acid sequence of SEQ ID NO: 73.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 72 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 73.

The antibodies or antigen-binding fragments thereof can comprise a heavy chain variable region (VH) comprising the amino acid sequence of SEQ ID NO: 72 and a light chain variable region (VL) comprising the amino acid sequence of SEQ ID NO: 73.

Provided herein is an anti-IL15 antibody or an antigen-binding portion thereof, wherein the antibody or antigen-binding portion comprises a VH region and a VL region, wherein (a) the VH region amino acid sequence comprises SEQ ID NO: 72; and the VL region amino acid sequence comprises SEQ ID NO: 73, with 1, 2 or 3 conservative amino acid substitutions in the VH region sequence, the VL region sequence, or both the VH region and the VL region sequences. In some embodiments, conservative amino acid substitutions are made only in the FR sequences and not in the CDR sequences of an antibody or antigen-binding portion.

Further provided herein are antibodies or antigen-binding fragments thereof that bind TSLP, wherein the antibody or antigen-binding fragment thereof comprises a heavy chain variable region (VH) and a light chain variable region (VL). In some embodiments, the antibody or antigen-binding fragment thereof comprises a VH with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 84. In some embodiments, the antibody or antigen-binding fragment thereof comprises a VL with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 85. In some embodiments, the antibody or antigen-binding fragment thereof comprises: a) a VH with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 84; and b) a VL with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 85. In some embodiments, the antibody or antigen-binding fragment thereof comprises: a) a VH with at least 90% sequence identity to SEQ ID NO: 84; and b) a VL with at least 90% sequence identity to SEQ ID NO: 85.

In some embodiments, the antibody or antigen-binding fragment thereof comprises: a heavy chain variable region (VH) comprising an amino acid sequence with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 84; and a light chain variable region (VL) with at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 85.

Further provided herein is an anti-TSLP antibody or an antigen-binding portion thereof that comprises a combination of a VH region amino acid sequence and VL region amino acid sequence, and/or a combination of CDR amino acid sequences provided in Table 5. In some embodiments, the antibody or antigen-binding fragment thereof comprises a VH comprising the amino acid sequence of SEQ ID NO: 84. In some embodiments, the antibody or antigen-binding fragment thereof comprises a VL comprising the amino acid sequence of SEQ ID NO: 85. In some embodiments, the antibody or antigen-binding fragment thereof comprises: a) a VH comprising the amino acid sequence of SEQ ID NO: 84; and b) a VL comprising the amino acid sequence of SEQ ID NO: 85. In some embodiments, the VH region amino acid sequence and/or the VL region amino acid sequence comprises a signal sequence (also known as a signal peptide) at the amino-terminus.

Disclosed herein are antibodies or antigen-binding fragments thereof that bind TSLP, wherein the antibodies or antigen-binding fragments thereof comprise: a heavy chain complementarity determining region 1 (HCDR1) comprising the amino acid sequence of SEQ ID NO: 76, a HCDR2 comprising the amino acid sequence of SEQ ID NO: 77, a HCDR3 comprising the amino acid sequence of SEQ ID NO: 78, a light chain complementarity determining region 1 (LCDR1) comprising the amino acid sequence of SEQ ID NO: 79, a LCDR2 comprising the amino acid sequence of SEQ ID NO: 80, and a LCDR3 comprising the amino acid sequence of SEQ ID NO: 81; wherein the numbering of HCDR1 is according to the EU index as in Kabat.

In some embodiments, the antibodies or antigen-binding fragments thereof comprise: a heavy chain complementarity determining region 1 (HCDR1) having at least 90% sequence identity with SEQ ID NO: 76, a HCDR2 having at least 90% sequence identity with SEQ ID NO: 77, a HCDR3 having at least 90% sequence identity with SEQ ID NO: 78, a light chain complementarity determining region 1 (LCDR1) having at least 90% sequence identity with SEQ ID NO: 79, a LCDR2 having at least 90% sequence identity with SEQ ID NO: 80, and a LCDR3 having at least 90% sequence identity with SEQ ID NO: 81; wherein the numbering of HCDR1 is according to the EU index as in Kabat.

The antibodies or antigen-binding fragments thereof can comprise a HCDR1 comprising the amino acid sequence of SEQ ID NO: 76, a HCDR2 comprising the amino acid sequence of SEQ ID NO: 77, a HCDR3 comprising the amino acid sequence of SEQ ID NO: 78, a LCDR1 comprising the amino acid sequence of SEQ ID NO: 79, a LCDR2 comprising the amino acid sequence of SEQ ID NO: 80, and a LCDR3 comprising the amino acid sequence of SEQ ID NO: 81.

Provided herein is an anti-TSLP antibody or an antigen-binding portion thereof, wherein the antibody or antigen-binding portion comprises a VH region and a VL region. In some embodiments, the VH region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 84. In some embodiments, the VH region comprises the amino acid sequence of SEQ ID NO: 84. In some embodiments, the VH region consists of the amino acid sequence of SEQ ID NO: 84.

In some embodiments, the VL region amino acid sequence comprises an amino acid sequence that has at least 90%, at least 91%, at least 92%, at least 93%, at least 94%, at least 95%, at least 96%, at least 97%, at least 98%, or at least 99% sequence identity to the amino acid sequence of SEQ ID NO: 85. In some embodiments, the VL region comprises the amino acid sequence of SEQ ID NO: 85. In some embodiments, the VL region consists of the amino acid sequence of SEQ ID NO: 85.

In some embodiments, the VH region amino acid sequence has at least 90%, at least 91%, at least 92%, at least 93%, at least 94%, at least 95%, at least 96%, at least 97%, at least 98%, or at least 99% sequence identity to the amino acid sequence of SEQ ID NO: 84 and the VL region amino acid sequence has at least 90% at least 91%, at least 92%, at least 93%, at least 94%, at least 95%, at least 96%, at least 97%, at least 98%, or at least 99% sequence identity to the amino acid sequence of SEQ ID NO: 85.

The antibodies or antigen-binding fragments thereof can comprise a heavy chain variable region (VH) comprising the amino acid sequence of SEQ ID NO: 84 and a light chain variable region (VL) comprising the amino acid sequence of SEQ ID NO: 85.

Provided herein is an anti-TSLP antibody or an antigen-binding portion thereof, wherein the antibody or antigen-binding portion comprises a VH region and a VL region, wherein (a) the VH region amino acid sequence comprises SEQ ID NO: 84; and the VL region amino acid sequence comprises SEQ ID NO: 85, with 1, 2 or 3 conservative amino acid substitutions in the VH region sequence, the VL region sequence, or both the VH region and the VL region sequences. In some embodiments, conservative amino acid substitutions are made only in the FR sequences and not in the CDR sequences of an antibody or antigen-binding portion.

In some embodiments, the antibody further comprises a region capable of binding a second antigen. In some embodiments, the second antigen is different from the first antigen. In some embodiments, the first antigen is any one of Activin-A, Alpha 2 Antiplasmin, Ang-2, ANGPTL4, ApoE4 mAb, APP mAb, APRIL, BAFF (BLyS)/APRIL, BDCA2, CCR7, CD122 (IL15Rβ), CD161, CD19, CD20, CD28, CD40L, CD47, CD47 (CVD), ChemR23, CTLA4, CX3CR1, DKK1, E7, EGFR, Factor, IIa, FcRn, FXIIa/FXIa, FZD4, G-CSFR, Galectin 9, GARP, ICOS, IGF-1R, IL13, IL15, IL17A, IL17F, IL18, IL18BP, IL1RAP, IL1β, IL2, IL21, IL23p19, IL25, IL31, IL33, IL36R, IL4Rα, IL7Ra, IL2Rg, ILT2/ILT4 (LILRB1/LILRB2), ILT3, ILT4 (LILRB2), Kallikrein 5/7, LGR5, LILRA4, LLT1, LRP5, MAdCAM-1, MAPT (Tau), Myostatin (GDF8), Netrin-1, OSMRP, OX40, OX40L, p40, PD-1, PD-L1, PSMA, Scl, SIRPα, TGF-β, TL1A, TNFα, TSLP, VEGF, VEGF-A, VEGF-C, VEGFR/C3b, YKL40 (CHI3L1), and α4β7. In some embodiments, the first antigen is any one of TL1A, IL1β, IL13, IL15, and TSLP. In some embodiments, the second antigen is any one of Activin-A, Alpha 2 Antiplasmin, Ang-2, ANGPTL4, ApoE4 mAb, APP mAb, APRIL, BAFF (BLyS)/APRIL, BDCA2, CCR7, CD122 (IL15Rβ), CD161, CD19, CD20, CD28, CD40L, CD47, CD47 (CVD), ChemR23, CTLA4, CX3CR1, DKK1, E7, EGFR, Factor, IIa, FcRn, FXIIa/FXIa, FZD4, G-CSFR, Galectin 9, GARP, ICOS, IGF-1R, IL13, IL15, IL17A, IL17F, IL18, IL18BP, IL1RAP, IL1β, IL2, IL21, IL23p19, IL25, IL31, IL33, IL36R, IL4Rα, IL7Ra, IL2Rg, ILT2/ILT4 (LILRB1/LILRB2), ILT3, ILT4 (LILRB2), Kallikrein 5/7, LGR5, LILRA4, LLT1, LRP5, MAdCAM-1, MAPT (Tau), Myostatin (GDF8), Netrin-1, OSMRP, OX40, OX40L, p40, PD-1, PD-L1, PSMA, Scl, SIRPα, TGF-β, TL1A, TNFα, TSLP, VEGF, VEGF-A, VEGF-C, VEGFR/C3b, YKL40 (CHI3L1), and α4β7. In some embodiments, the second antigen is any one of TL1A, IL1β, IL13, IL15, and TSLP. In some embodiments, the first antigen is IL13. In some embodiments, the second antigen is TSLP, and the region capable of binding a second antigen comprises a heavy chain variable region (VH) comprising an amino acid sequence with at least 90% sequence identity to SEQ ID NO: 84 and a light chain variable region (VL) comprising an amino acid sequence with at least 90% sequence identity to SEQ ID NO: 85. In some embodiments, the region capable of binding a second antigen comprises a heavy chain complementarity determining region 1 (HCDR1) having at least 90% sequence identity with SEQ ID NO: 76, a HCDR2 having at least 90% sequence identity with SEQ ID NO: 77, a HCDR3 having at least 90% sequence identity with SEQ ID NO: 78, a light chain complementarity determining region 1 (LCDR1) having at least 90% sequence identity with SEQ ID NO: 79, a LCDR2 having at least 90% sequence identity with SEQ ID NO: 80, and a LCDR3 having at least 90% sequence identity with SEQ ID NO: 81. In some embodiments, the region capable of binding a second antigen comprises a heavy chain variable region (VH) comprising the amino acid sequence of SEQ ID NO: 84 and a light chain variable region (VL) comprising the amino acid sequence of SEQ ID NO: 85.

In some embodiments, an antibody or antigen-binding portion provided herein (e.g., an anti-TL1A antibody or antigen-binding portion) is monoclonal. The term “monoclonal antibody” (Mab) refers to an antibody, or antigen-binding portion thereof, that is derived from a single copy or clone, including for example any eukaryotic, prokaryotic, or phage clone, and not the method by which it is produced. Preferably, a monoclonal antibody exists in a homogeneous or substantially homogeneous population.

In some embodiments, the antibody or antigen-binding portion provided herein may be isolated.

In some embodiments, an antibody or antigen-binding portion provided herein (e.g., an anti-TL1A antibody or antigen-binding portion) is chimeric. The term “chimeric” is intended to refer to an antibody molecule, or an antigen-binding portion thereof, in which the variable domain sequences are derived from one species and at least one constant region sequence is derived from another species. For example, one or all the variable domains of the light chain(s) and/or one or all the variable domains of the heavy chain(s) of a mouse antibody (e.g., a mouse monoclonal antibody) may each be joined to a human constant region, such as, without limitation an IgG1 or an IgG4 human constant region. Examples of chimeric antibodies and suitable techniques for their generation are provided in U.S. Pat. Nos. 4,816,567; 4,975,369; and 4,816,397, each of which is incorporated herein by reference in its entirety. In some embodiments, an anti-TL1A antibody or an antigen-binding portion provided herein comprises: (a) a VH region amino acid sequence comprising SEQ ID NO: 7; a VL region amino acid sequence comprising SEQ ID NO: 8 and a human constant region; (b) a VH region amino acid sequence comprising SEQ ID NO: 13, a VL region amino acid sequence comprising SEQ ID NO: 14 and a human constant region; (c) a VH comprising the amino acid sequence of SEQ ID NO: 7, a VL comprising the amino acid sequence of SEQ ID NO: 20 and a human constant region; (d) a VH region amino acid sequence comprising SEQ ID NO: 7; a VL region amino acid sequence comprising SEQ ID NO: 20 and a human constant region; (e) a VH region amino acid sequence comprising SEQ ID NO: 23, a VL region amino acid sequence comprising SEQ ID NO: 20 and a human constant region; or (f) a VH comprising the amino acid sequence of SEQ ID NO: 28, a VL comprising the amino acid sequence of SEQ ID NO: 20 and a human constant region. In some embodiments, an anti-IL1P antibody or an antigen-binding portion provided herein comprises: (a) a VH region amino acid sequence comprising SEQ ID NO: 49; a VL region amino acid sequence comprising SEQ ID NO: 50 and a human constant region. In some embodiments, an anti-IL13 antibody or an antigen-binding portion provided herein comprises: (a) a VH region amino acid sequence comprising SEQ ID NO: 59; a VL region amino acid sequence comprising SEQ ID NO: 60 and a human constant region; or (b) a VH region amino acid sequence comprising SEQ ID NO: 86, a VL region amino acid sequence comprising SEQ ID NO: 87 and a human constant region. In some embodiments, an anti-TSLP antibody or an antigen-binding portion provided herein comprises: (a) a VH region amino acid sequence comprising SEQ ID NO: 84; a VL region amino acid sequence comprising SEQ ID NO: 85 and a human constant region. In some embodiments, an anti-IL15 antibody or an antigen-binding portion provided herein comprises: (a) a VH region amino acid sequence comprising SEQ ID NO: 72; a VL region amino acid sequence comprising SEQ ID NO: 73 and a human constant region.

In some embodiments, an antibody or antigen-binding portion provided herein (e.g., an anti-TL1A antibody or antigen-binding portion) is humanized. The term “humanized” is intended to refer to an antibody, or an antigen-binding portion thereof, that has been engineered to comprise one or more human framework regions in the variable domain together with non-human (e.g., mouse, rat, or hamster) CDRs of the heavy and/or light chain. In some embodiments, a humanized antibody comprises sequences that are entirely human except for the CDRs. For example, an antibody molecule or antigen-binding portion thereof (e.g., an anti-TL1A antibody or antigen-binding portion thereof) may comprise one or more human variable region framework scaffolds into which the CDRs have been inserted. In some embodiments, the VH region, the VL region, or both the VH region and the VL region of an antibody or antigen-binding portion (e.g., an anti-TL1A antibody or antigen-binding portion) provided herein comprise one or more human framework region amino acid sequences. In some embodiments, a humanized antibody comprises sequences that are entirely human except for the CDRs, which are the CDRs of antibody AB_TL1A_02_004, AB_TL1A_01_005, AB_TL1A_02_002, AB_TL1A_02_005, AB_TL1A_02_009, AB_TL1A_HL2_, or AB_TL1A_HL9_002. Examples of humanized antibodies and suitable techniques for their generation are provided in Hwang et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and WO 90/07861, each of which is incorporated herein by reference in its entirety. When choosing FR to flank CDRs, for example when humanizing or optimizing an antibody, FRs from antibodies that contain CDR sequences in the same canonical class are preferred.

Also provided herein is an anti-TL1A antibody or an antigen-binding portion thereof, wherein the antibody or antigen-binding portion comprises a VH region, a VL region and all human framework region sequences, wherein: (a) the VH region amino acid sequence comprises a HCDR1 of SEQ ID NO: 25, a HCDR2 of SEQ ID NO: 2 and a HCDR3 of SEQ ID NO: 3; and the VL region amino acid sequence comprises a LCDR1 of SEQ ID NO: 4, a LCDR2 of SEQ ID NO: 5 and a LCDR3 of SEQ ID NO: 6; (b) the VH region amino acid sequence comprises a HCDR1 of SEQ ID NO: 25, a HCDR2 of SEQ ID NO: 11 and a HCDR3 of SEQ ID NO: 3; and the VL region amino acid sequence comprises a LCDR1 of SEQ ID NO: 4, a LCDR2 of SEQ ID NO: 5 and a LCDR3 of SEQ ID NO: 12; (c) the VH region amino acid sequence comprises a HCDR1 of SEQ ID NO: 25, a HCDR2 of SEQ ID NO: 2 and a HCDR3 of SEQ ID NO: 3; and the VL region amino acid sequence comprises a LCDR1 of SEQ ID NO: 4, a LCDR2 of SEQ ID NO: 17 and a LCDR3 of SEQ ID NO: 6; (d) the VH region amino acid sequence comprises a HCDR1 of SEQ ID NO: 25, a HCDR2 of SEQ ID NO: 2 and a HCDR3 of SEQ ID NO: 3; and the VL region amino acid sequence comprises a LCDR1 of SEQ ID NO: 4, a LCDR2 of SEQ ID NO: 17 and a LCDR3 of SEQ ID NO: 12; (e) the VH region amino acid sequence comprises a HCDR1 of SEQ ID NO: 25, a HCDR2 of SEQ ID NO: 22 and a HCDR3 of SEQ ID NO: 3; and the VL region amino acid sequence comprises a LCDR1 of SEQ ID NO: 4, a LCDR2 of SEQ ID NO: 17 and a LCDR3 of SEQ ID NO: 12; (f) the VH region amino acid sequence comprises a HCDR1 of SEQ ID NO: 25, a HCDR2 of SEQ ID NO: 2 and a HCDR3 of SEQ ID NO: 3; and the VL region amino acid sequence comprises a LCDR1 of SEQ ID NO: 4, a LCDR2 of SEQ ID NO: 5 and a LCDR3 of SEQ ID NO: 6; or (g) the VH region amino acid sequence comprises a HCDR1 of SEQ ID NO: 25, a HCDR2 of SEQ ID NO: 2 and a HCDR3 of SEQ ID NO: 27; and the VL region amino acid sequence comprises a LCDR1 of SEQ ID NO: 4, a LCDR2 of SEQ ID NO: 5 and a LCDR3 of SEQ ID NO: 6. Also provided herein is an anti-TL1A antibody or an antigen-binding portion thereof, wherein the antibody or antigen-binding portion comprises a VH region, a VL region and one or more human framework region sequences, wherein: (a) the VH region amino acid sequence comprises a HCDR1 of SEQ ID NO: 25, a HCDR2 of SEQ ID NO: 2 and a HCDR3 of SEQ ID NO: 3; and the VL region amino acid sequence comprises a LCDR1 of SEQ ID NO: 4, a LCDR2 of SEQ ID NO: 5 and a LCDR3 of SEQ ID NO: 6; (b) the VH region amino acid sequence comprises a HCDR1 of SEQ ID NO: 25, a HCDR2 of SEQ ID NO: 11 and a HCDR3 of SEQ ID NO: 3; and the VL region amino acid sequence comprises a LCDR1 of SEQ ID NO: 4, a LCDR2 of SEQ ID NO: 5 and a LCDR3 of SEQ ID NO: 12; (c) the VH region amino acid sequence comprises a HCDR1 of SEQ ID NO: 25, a HCDR2 of SEQ ID NO: 2 and a HCDR3 of SEQ ID NO: 3; and the VL region amino acid sequence comprises a LCDR1 of SEQ ID NO: 4, a LCDR2 of SEQ ID NO: 17 and a LCDR3 of SEQ ID NO: 6; (d) the VH region amino acid sequence comprises a HCDR1 of SEQ ID NO: 25, a HCDR2 of SEQ ID NO: 2 and a HCDR3 of SEQ ID NO: 3; and the VL region amino acid sequence comprises a LCDR1 of SEQ ID NO: 4, a LCDR2 of SEQ ID NO: 17 and a LCDR3 of SEQ ID NO: 12; (e) the VH region amino acid sequence comprises a HCDR1 of SEQ ID NO: 25, a HCDR2 of SEQ ID NO: 22 and a HCDR3 of SEQ ID NO: 3; and the VL region amino acid sequence comprises a LCDR1 of SEQ ID NO: 4, a LCDR2 of SEQ ID NO: 17 and a LCDR3 of SEQ ID NO: 12; (f) the VH region amino acid sequence comprises a HCDR1 of SEQ ID NO: 25, a HCDR2 of SEQ ID NO: 2 and a HCDR3 of SEQ ID NO: 3; and the VL region amino acid sequence comprises a LCDR1 of SEQ ID NO: 4, a LCDR2 of SEQ ID NO: 5 and a LCDR3 of SEQ ID NO: 6; or (g) the VH region amino acid sequence comprises a HCDR1 of SEQ ID NO: 25, a HCDR2 of SEQ ID NO: 2 and a HCDR3 of SEQ ID NO: 27; and the VL region amino acid sequence comprises a LCDR1 of SEQ ID NO: 4, a LCDR2 of SEQ ID NO: 5 and a LCDR3 of SEQ ID NO: 6.

Also provided herein is an anti-IL1P antibody or an antigen-binding portion thereof, wherein the antibody or antigen-binding portion comprises a VH region, a VL region and all human framework region sequences, wherein: the VH region amino acid sequence comprises a HCDR1 of SEQ ID NO: 43, a HCDR2 of SEQ ID NO: 44 and a HCDR3 of SEQ ID NO: 45; and the VL region amino acid sequence comprises a LCDR1 of SEQ ID NO: 46, a LCDR2 of SEQ ID NO: 47 and a LCDR3 of SEQ ID NO: 48.

Also provided herein is an anti-IL1P antibody or an antigen-binding portion thereof, wherein the antibody or antigen-binding portion comprises a VH region, a VL region and one or more human framework region sequences, wherein: the VH region amino acid sequence comprises a HCDR1 of SEQ ID NO: 43, a HCDR2 of SEQ ID NO: 44 and a HCDR3 of SEQ ID NO: 45; and the VL region amino acid sequence comprises a LCDR1 of SEQ ID NO: 46, a LCDR2 of SEQ ID NO: 47 and a LCDR3 of SEQ ID NO: 48.

Also provided herein is an anti-IL13 antibody or an antigen-binding portion thereof, wherein the antibody or antigen-binding portion comprises a VH region, a VL region and all human framework region sequences, wherein: the VH region amino acid sequence comprises a HCDR1 of SEQ ID NO: 53, a HCDR2 of SEQ ID NO: 54 and a HCDR3 of SEQ ID NO: 55; and the VL region amino acid sequence comprises a LCDR1 of SEQ ID NO: 56, a LCDR2 of SEQ ID NO: 57 and a LCDR3 of SEQ ID NO: 58; or (b) the VH region amino acid sequence comprises a HCDR1 of SEQ ID NO: 53, a HCDR2 of SEQ ID NO: 54 and a HCDR3 of SEQ ID NO: 82; and the VL region amino acid sequence comprises a LCDR1 of SEQ ID NO: 56, a LCDR2 of SEQ ID NO: 57 and a LCDR3 of SEQ ID NO: 58.

Also provided herein is an anti-IL13 antibody or an antigen-binding portion thereof, wherein the antibody or antigen-binding portion comprises a VH region, a VL region and one or more human framework region sequences, wherein: the VH region amino acid sequence comprises a HCDR1 of SEQ ID NO: 53, a HCDR2 of SEQ ID NO: 54 and a HCDR3 of SEQ ID NO: 55; and the VL region amino acid sequence comprises a LCDR1 of SEQ ID NO: 56, a LCDR2 of SEQ ID NO: 57 and a LCDR3 of SEQ ID NO: 58; or (b) the VH region amino acid sequence comprises a HCDR1 of SEQ ID NO: 53, a HCDR2 of SEQ ID NO: 54 and a HCDR3 of SEQ ID NO: 82; and the VL region amino acid sequence comprises a LCDR1 of SEQ ID NO: 56, a LCDR2 of SEQ ID NO: 57 and a LCDR3 of SEQ ID NO: 58.

Also provided herein is an anti-TSLP antibody or an antigen-binding portion thereof, wherein the antibody or antigen-binding portion comprises a VH region, a VL region and all human framework region sequences, wherein: the VH region amino acid sequence comprises a HCDR1 of SEQ ID NO: 76, a HCDR2 of SEQ ID NO: 77 and a HCDR3 of SEQ ID NO: 78; and the VL region amino acid sequence comprises a LCDR1 of SEQ ID NO: 79, a LCDR2 of SEQ ID NO: 80 and a LCDR3 of SEQ ID NO: 81.

Also provided herein is an anti-TSLP antibody or an antigen-binding portion thereof, wherein the antibody or antigen-binding portion comprises a VH region, a VL region and one or more human framework region sequences, wherein: the VH region amino acid sequence comprises a HCDR1 of SEQ ID N076, a HCDR2 of SEQ ID NO: 77 and a HCDR3 of SEQ ID NO: 78; and the VL region amino acid sequence comprises a LCDR1 of SEQ ID NO: 79, a LCDR2 of SEQ ID NO: 80 and a LCDR3 of SEQ ID NO: 81.

Also provided herein is an anti-IL15 antibody or an antigen-binding portion thereof, wherein the antibody or antigen-binding portion comprises a VH region, a VL region and all human framework region sequences, wherein: the VH region amino acid sequence comprises a HCDR1 of SEQ ID NO: 66, a HCDR2 of SEQ ID NO: 67 and a HCDR3 of SEQ ID NO: 68; and the VL region amino acid sequence comprises a LCDR1 of SEQ ID NO: 69, a LCDR2 of SEQ ID NO: 70 and a LCDR3 of SEQ ID NO: 71.

Also provided herein is an anti-IL15 antibody or an antigen-binding portion thereof, wherein the antibody or antigen-binding portion comprises a VH region, a VL region and one or more human framework region sequences, wherein: the VH region amino acid sequence comprises a HCDR1 of SEQ ID NO: 66, a HCDR2 of SEQ ID NO: 67 and a HCDR3 of SEQ ID NO: 68; and the VL region amino acid sequence comprises a LCDR1 of SEQ ID NO: 69, a LCDR2 of SEQ ID NO: 70 and a LCDR3 of SEQ ID NO: 71.

In some embodiments, an antibody or an antigen-binding portion thereof provided herein (e.g., an anti-TL1A antibody or antigen-binding portion) comprises an immunoglobulin constant region. In some embodiments, the immunoglobulin constant region is a wild type immunoglobulin constant region. In some embodiments, the immunoglobulin constant region is a variant immunoglobulin constant region.

In some embodiments, the immunoglobulin constant region is an IgG, IgE, IgM, IgD, IgA or IgY constant region. In some embodiments, the immunoglobulin constant region is an IgG1, IgG2, IgG3, IgG4, IgAQ1 or IgA2 constant region. In some embodiments, the immunoglobulin constant region is an IgG1 constant region. In some embodiments, the immunoglobulin constant region is an IgG4 constant region

In some embodiments, the immunoglobulin constant region comprises one or more mutations to increase half-life. In some embodiments, the immunoglobulin constant region is immunologically inert. In some embodiments, the immunoglobulin constant region comprises one or more mutations to increase FcRn binding, antibody-dependent cell-mediated cytotoxicity activity, and/or complement-dependent cytotoxicity activity.

In some embodiments, the immunoglobulin constant region is a wild-type human IgG1 constant region comprising a L234A substitution, a L235A substitution, a G237A substitution, a Q311R substitution, a D356E substitution, a L358M substitution, a M428L substitution, a H433K substitution, a N434S substitution, a N434F substitution or a combination thereof, wherein numbering is according to the EU index as in Kabat.

In some embodiments, the immunoglobulin constant region is a wild-type human IgG4 constant region comprising a F234A substitution, a L235A substitution, a G237A substitution, Q311R substitution, a D356E substitution, a L358M substitution, a M428L substitution, a H433K substitution, a N434S substitution, a N434F substitution, or a combination thereof, wherein numbering is according to the EU index as in Kabat.

In some embodiments, the immunoglobulin constant region is a wild-type human IgG1 constant region comprising a L234A substitution, a L235A substitution, a G237A substitution, a D356E substitution, and a L358M substitution, wherein numbering is according to the EU index as in Kabat. In some embodiments, the immunoglobulin constant region is a wild-type human IgG4 constant region comprising a F234A substitution, a L235A substitution, a G237A substitution, a D356E substitution, and a L358M substitution, wherein numbering is according to the EU index as in Kabat.

In some embodiments, the immunoglobulin constant region is a wild-type human IgG1 or IgG4 constant region comprising a M428L substitution and a N434S substitution, wherein numbering is according to the EU index as in Kabat.

In some embodiments, the immunoglobulin constant region is a wild-type human IgG1 or IgG4 constant region comprising a Q311R substitution, a M428L substitution, and a N434F substitution, wherein numbering is according to the EU index as in Kabat. In some embodiments, the immunoglobulin constant region is a wild-type human IgG1 or IgG4 constant region comprising a M428L substitution, a H433K substitution, and a N434F substitution, wherein numbering is according to the EU index as in Kabat.

In some embodiments, the immunoglobulin constant region is a wild-type human IgG1 or IgG4 constant region comprising a Q311R substitution, a M428L substitution, a H433K substitution, and a N434F substitution, wherein numbering is according to the EU index as in Kabat.

In some embodiments, the antibodies or antigen-binding fragments thereof can comprise a L234A substitution, a L235A substitution, a G237A substitution, a D356E substitution, a L358M substitution, or a combination thereof. In some embodiments, the antibodies or antigen-binding fragments thereof can comprise a L234A substitution and a L235A substitution (“LALA mutations”). In some embodiments, the antibodies or antigen-binding fragments thereof can comprise a L234A substitution, a L235A substitution, a G237A substitution, a D356E substitution, and a L358M substitution.

In some embodiments, a position of an amino acid residue in a constant region of an immunoglobulin molecule is numbered according to the EU index as in Kabat (Ward et al., 1995 Therap. Immunol. 2:77-94).

In some embodiments, an antibody or an antigen-binding portion thereof provided herein (e.g., an anti-TL1A antibody or antigen-binding portion) may comprise an immunoglobulin light chain constant region that is a kappa light chain constant region or a lambda light chain constant region.

In some embodiments, an antibody or an antigen-binding portion thereof provided herein (e.g., an anti-TL1A antibody or antigen-binding portion) may comprise an immunoglobulin constant region comprising any one of the amino acid sequences in Table 7. The immunoglobulin constant region sequences in Table 7 begin at the CH1 domain. In some embodiments, an antibody or an antigen-binding portion thereof (e.g., an anti-TL1A antibody or antigen-binding portion) provided herein may comprise an immunoglobulin heavy chain constant region comprising any one of SEQ ID NOs: 33-35, 95-98, and 128-132. In some embodiments, an antibody or an antigen-binding portion thereof (e.g., an anti-TL1A antibody or antigen-binding portion) provided herein may comprise an immunoglobulin light chain constant region comprising SEQ ID NO: 36.

In some embodiments, an anti-TL1A antibody or antigen-binding portion provided herein may comprise an immunoglobulin heavy chain constant region comprising SEQ ID NO: 33; and an immunoglobulin light chain constant region comprising SEQ ID NO: 36. In some embodiments, an anti-TL1A antibody or antigen-binding portion provided herein may comprise an immunoglobulin heavy chain constant region comprising SEQ ID NO: 34; and an immunoglobulin light chain constant region comprising SEQ ID NO: 36. In some embodiments, an anti-TL1A antibody or antigen-binding portion provided herein may comprise an immunoglobulin heavy chain constant region comprising SEQ ID NO: 35; and an immunoglobulin light chain constant region comprising SEQ ID NO: 36. In some embodiments, an anti-TL1A antibody or antigen-binding portion provided herein may comprise an immunoglobulin heavy chain constant region comprising SEQ ID NO: 95; and an immunoglobulin light chain constant region comprising SEQ ID NO: 36. In some embodiments, an anti-TL1A antibody or antigen-binding portion provided herein may comprise an immunoglobulin heavy chain constant region comprising SEQ ID NO: 96; and an immunoglobulin light chain constant region comprising SEQ ID NO: 36. In some embodiments, an anti-TL1A antibody or antigen-binding portion provided herein may comprise an immunoglobulin heavy chain constant region comprising SEQ ID NO: 97; and an immunoglobulin light chain constant region comprising SEQ ID NO: 36. In some embodiments, an anti-TL1A antibody or antigen-binding portion provided herein may comprise an immunoglobulin heavy chain constant region comprising SEQ ID NO: 98; and an immunoglobulin light chain constant region comprising SEQ ID NO: 36.

In some embodiments, an anti-IL1P antibody or antigen-binding portion provided herein may comprise an immunoglobulin heavy chain constant region comprising SEQ ID NO: 128; and an immunoglobulin light chain constant region comprising SEQ ID NO: 36.

In some embodiments, an anti-IL13 antibody or antigen-binding portion provided herein may comprise an immunoglobulin heavy chain constant region comprising SEQ ID NO: 129; and an immunoglobulin light chain constant region comprising SEQ ID NO: 36. In some embodiments, an anti-IL13 antibody or antigen-binding portion provided herein may comprise an immunoglobulin heavy chain constant region comprising SEQ ID NO: 130; and an immunoglobulin light chain constant region comprising SEQ ID NO: 36.

In some embodiments, an anti-TSLP antibody or antigen-binding portion provided herein may comprise an immunoglobulin heavy chain constant region comprising SEQ ID NO: 131; and an immunoglobulin light chain constant region comprising SEQ ID NO: 36.

In some embodiments, an anti-IL15 antibody or antigen-binding portion provided herein may comprise an immunoglobulin heavy chain constant region comprising SEQ ID NO: 1; and an immunoglobulin light chain constant region comprising SEQ ID NO: 36.

In some embodiments, an anti-TL1A antibody or an antigen-binding portion thereof provided herein may comprise an immunoglobulin constant region comprising (i) any one of SEQ ID NOs: 33-35, 95-98, and 128-132; and (ii) SEQ ID NO: 36. In some embodiments, an anti-TL1A antibody or an antigen-binding portion thereof provided herein may comprise an immunoglobulin constant region comprising SEQ ID NO 33; and SEQ ID NO: 36.

In some embodiments, an anti-IL1P antibody or an antigen-binding portion thereof provided herein may comprise an immunoglobulin constant region comprising SEQ ID NO 128; and SEQ ID NO: 36. In some embodiments, an anti-IL13 antibody or an antigen-binding portion thereof provided herein may comprise an immunoglobulin constant region comprising SEQ ID NO 129; and SEQ ID NO: 36. In some embodiments, an anti-IL13 antibody or an antigen-binding portion thereof provided herein may comprise an immunoglobulin constant region comprising SEQ ID NO 130; and SEQ ID NO: 36. In some embodiments, an anti-TSLP antibody or an antigen-binding portion thereof provided herein may comprise an immunoglobulin constant region comprising SEQ ID NO 131; and SEQ ID NO: 36. In some embodiments, an anti-IL15 antibody or an antigen-binding portion thereof provided herein may comprise an immunoglobulin constant region comprising SEQ ID NO 132; and SEQ ID NO: 36.

In some embodiments, an antibody or an antigen-binding portion thereof provided herein may comprise the six CDR amino acid sequences of any one of the clones in Table 1 and any one of the immunoglobulin heavy chain constant region amino acid sequences in Table 7. In some embodiments, an antibody or an antigen-binding portion thereof provided herein may comprise an immunoglobulin heavy chain constant region comprising any one of the immunoglobulin heavy chain constant region amino acid sequences in Table 7 and an immunoglobulin light chain constant region that is a kappa light chain constant region or a lambda light chain constant region.

In some embodiments, provided herein is an anti-TL1A antibody or an antigen-binding portion thereof, wherein the antibody comprises a VH region, a VL region, a heavy chain constant region, and a light chain constant region, wherein (a) the VH region amino acid sequence comprises a HCDR1 comprising SEQ ID NO: 25, a HCDR2 comprising SEQ ID NO: 2 and a HCDR3 comprising SEQ ID NO: 3; the VL region amino acid sequence comprises a LCDR1 comprising SEQ ID NO: 4, a LCDR2 comprising SEQ ID NO: 5 and a LCDR3 comprising SEQ ID NO: 6; the heavy chain constant region comprises any one of SEQ ID NOS: 33-35, 95-98, and 128-132; and the light chain constant region comprises SEQ ID NO: 36; (b) the VH region amino acid sequence comprises a HCDR1 comprising SEQ ID NO: 25, a HCDR2 comprising SEQ ID NO: 11 and a HCDR3 comprising SEQ ID NO: 3; the VL region amino acid sequence comprises a LCDR1 comprising SEQ ID NO: 4, a LCDR2 comprising SEQ ID NO: 5 and a LCDR3 comprising SEQ ID NO: 12; the heavy chain constant region comprises any one of SEQ ID NOS: 33-35, 95-98, and 128-132; and the light chain constant region comprises SEQ ID NO: 36; (c) the VH region amino acid sequence comprises a HCDR1 comprising SEQ ID NO: 25, a HCDR2 comprising SEQ ID NO: 2 and a HCDR3 comprising SEQ ID NO: 3; the VL region amino acid sequence comprises a LCDR comprising SEQ ID NO: 4, a LCDR2 comprising SEQ ID NO: 17 and a LCDR3 comprising SEQ ID NO: 6; the heavy chain constant region comprises any one of SEQ ID NOS: the heavy chain constant region comprises any one of SEQ ID NOS: 33-35, 95-98, and 128-132; and the light chain constant region comprises SEQ ID NO: 36; (d) the VH region amino acid sequence comprises a HCDR1 comprising SEQ ID NO: 25, a HCDR2 o comprising SEQ ID NO: 2 and a HCDR3 comprising SEQ ID NO: 3; the VL region amino acid sequence comprises a LCDR1 comprising SEQ ID NO: 4, a LCDR2 comprising SEQ ID NO: 17 and a LCDR3 comprising SEQ ID NO: 12; the heavy chain constant region comprises any one of SEQ ID NOS: the heavy chain constant region comprises any one of SEQ ID NOS: 33-35, 95-98, and 128-132; and the light chain constant region comprises SEQ ID NO: 36; (e) the VH region amino acid sequence comprises a HCDR1 comprising SEQ ID NO: 25, a HCDR2 comprising SEQ ID NO: 22 and a HCDR3 comprising SEQ ID NO: 3; the VL region amino acid sequence comprises a LCDR1 comprising SEQ ID NO: 4, a LCDR2 comprising SEQ ID NO: 17 and a LCDR3 comprising SEQ ID NO: 12; the heavy chain constant region comprises any one of SEQ ID NOS: the heavy chain constant region comprises any one of SEQ ID NOS: 33-35, 95-98, and 128-132; and the light chain constant region comprises SEQ ID NO: 36; (f) the VH region amino acid sequence comprises a HCDR1 comprising SEQ ID NO: 25, a HCDR2 comprising SEQ ID NO: 2 and a HCDR3 comprising SEQ ID NO: 3; the VL region amino acid sequence comprises a LCDR1 comprising SEQ ID NO: 4, a LCDR2 comprising SEQ ID NO: 5 and a LCDR3 comprising SEQ ID NO: 6; the heavy chain constant region comprises any one of SEQ ID NOS: 33-35, 95-98, and 128-132; and the light chain constant region comprises SEQ ID NO: 36; or (g) the VH region amino acid sequence comprises a HCDR1 comprising SEQ ID NO: 25, a HCDR2 comprising SEQ ID NO: 2 and a HCDR3 comprising SEQ ID NO: 27; the VL region amino acid sequence comprises a LCDR1 comprising SEQ ID NO: 4, a LCDR2 comprising SEQ ID NO: 5 and a LCDR3 comprising SEQ ID NO: 6; the heavy chain constant region comprises any one of SEQ ID NOS: 33-35, 95-98, and 128-132; and the light chain constant region comprises SEQ ID NO: 36.

In some embodiments, provided herein is an anti-IL13 antibody or an antigen-binding portion thereof, wherein the antibody comprises a VH region, a VL region, a heavy chain constant region, and a light chain constant region, wherein the VH region amino acid sequence comprises a HCDR1 comprising SEQ ID NO: 43, a HCDR2 comprising SEQ ID NO: 44 and a HCDR3 comprising SEQ ID NO: 45; the VL region amino acid sequence comprises a LCDR1 comprising SEQ ID NO: 46, a LCDR2 comprising SEQ ID NO: 47, and a LCDR3 comprising SEQ ID NO: 48; the heavy chain constant region comprises any one of SEQ ID NOS: 33-35, 95-98, and 128-132; and the light chain constant region comprises SEQ ID NO: 36.

In some embodiments, provided herein is an anti-IL13 antibody or an antigen-binding portion thereof, wherein the antibody comprises a VH region, a VL region, a heavy chain constant region, and a light chain constant region, wherein a) the VH region amino acid sequence comprises a HCDR1 comprising SEQ ID NO: 53, a HCDR2 comprising SEQ ID NO: 54 and a HCDR3 comprising SEQ ID NO: 55; the VL region amino acid sequence comprises a LCDR1 comprising SEQ ID NO: 56, a LCDR2 comprising SEQ ID NO: 57, and a LCDR3 comprising SEQ ID NO: 58; the heavy chain constant region comprises any one of SEQ ID NOS: 33-35, 95-98, and 128-132; and the light chain constant region comprises SEQ ID NO: 36; or b) the VH region amino acid sequence comprises a HCDR1 comprising SEQ ID NO: 53, a HCDR2 comprising SEQ ID NO: 54 and a HCDR3 comprising SEQ ID NO: 63; the VL region amino acid sequence comprises a LCDR1 comprising SEQ ID NO: 56, a LCDR2 comprising SEQ ID NO: 57, and a LCDR3 comprising SEQ ID NO: 58; the heavy chain constant region comprises any one of SEQ ID NOS: 33-35, 95-98, and 128-132; and the light chain constant region comprises SEQ ID NO: 36.

In some embodiments, provided herein is an anti-IL15 antibody or an antigen-binding portion thereof, wherein the antibody comprises a VH region, a VL region, a heavy chain constant region, and a light chain constant region, wherein the VH region amino acid sequence comprises a HCDR1 comprising SEQ ID NO: 66, a HCDR2 comprising SEQ ID NO: 67 and a HCDR3 comprising SEQ ID NO: 68; the VL region amino acid sequence comprises a LCDR1 comprising SEQ ID NO: 69, a LCDR2 comprising SEQ ID NO: 70, and a LCDR3 comprising SEQ ID NO: 71; the heavy chain constant region comprises any one of SEQ ID NOS: 33-35, 95-98, and 128-132; and the light chain constant region comprises SEQ ID NO: 36.

In some embodiments, provided herein is an anti-TSLP antibody or an antigen-binding portion thereof, wherein the antibody comprises a VH region, a VL region, a heavy chain constant region, and a light chain constant region, wherein the VH region amino acid sequence comprises a HCDR1 comprising SEQ ID NO: 76, a HCDR2 comprising SEQ ID NO: 77 and a HCDR3 comprising SEQ ID NO: 78; the VL region amino acid sequence comprises a LCDR1 comprising SEQ ID NO: 79, a LCDR2 comprising SEQ ID NO: 80, and a LCDR3 comprising SEQ ID NO: 81; the heavy chain constant region comprises any one of SEQ ID NOS: 33-35, 95-98, and 128-132; and the light chain constant region comprises SEQ ID NO: 36.

In some embodiments, provided herein is an bispecific anti-IL13 anti-TSLP antibody or an antigen-binding portion thereof, wherein the antibody comprises: a) a first VH region, a first VL region, a heavy chain constant region, and a light chain constant region, and b) an ScFv-Fc comprising a second VL region, a second VH region, and an Fc region; wherein the first VH region amino acid sequence comprises a HCDR1 comprising SEQ ID NO: 76, a HCDR2 comprising SEQ ID NO: 77 and a HCDR3 comprising SEQ ID NO: 78; the first VL region amino acid sequence comprises a LCDR1 comprising SEQ ID NO: 79, a LCDR2 comprising SEQ ID NO: 80, and a LCDR3 comprising SEQ ID NO: 81; the heavy chain constant region comprises any one of SEQ ID NOS: 33-35, 95-98, and 128-132; the light chain constant region comprises SEQ ID NO: 36; the second VL region comprises a LCDR1 comprising SEQ ID NO: 56, a LCDR2 comprising SEQ ID NO: 57, and a LCDR3 comprising SEQ ID NO: 58; the second VH region comprises a HCDR1 comprising SEQ ID NO: 53, a HCDR2 comprising SEQ ID NO: 54 and a HCDR3 comprising SEQ ID NO: 63; and the Fc region comprises SEQ ID NO: 90.

The antibodies or antigen-binding fragments thereof can comprise:
a) a heavy chain (HC) comprising the amino acid sequence of SEQ ID NO: 9 and a light chain (LC) comprising the amino acid sequence of SEQ ID NO: 10;
b) a HC comprising the amino acid sequence of SEQ ID NO: 15 and a LC comprising the amino acid sequence of SEQ ID NO: 16;
c) a HC comprising the amino acid sequence of SEQ ID NO: 9 and a LC comprising the amino acid sequence of SEQ ID NO: 19;
d) a HC comprising the amino acid sequence of SEQ ID NO: 9 and a LC comprising the amino acid sequence of SEQ ID NO: 21; or
e) a HC comprising the amino acid sequence of SEQ ID NO: 24 and a LC comprising the amino acid sequence of SEQ ID NO: 21.

The antibodies or antigen-binding fragments thereof can comprise a HC comprising the amino acid sequence of SEQ ID NO: 9 and a LC comprising the amino acid sequence of SEQ ID NO: 10.

The antibodies or antigen-binding fragments thereof can comprise a HC comprising the amino acid sequence of SEQ ID NO: 15 and a LC comprising the amino acid sequence of SEQ ID NO: 16.

The antibodies or antigen-binding fragments thereof can comprise a HC comprising the amino acid sequence of SEQ ID NO: 9 and a LC comprising the amino acid sequence of SEQ ID NO: 19.

The antibodies or antigen-binding fragments thereof can comprise a HC comprising the amino acid sequence of SEQ ID NO: 9 and a LC comprising the amino acid sequence of SEQ ID NO: 21.

The antibodies or antigen-binding fragments thereof can comprise a HC comprising the amino acid sequence of SEQ ID NO: 24 and a LC comprising the amino acid sequence of SEQ ID NO: 21.

Disclosed herein are nucleic acid molecules encoding any of the herein disclosed antibodies or antigen-binding fragments thereof. In some embodiments, the nucleic acid molecules encode an antibody or antigen-binding fragment thereof that comprises:
a) a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6;
b) a HCDR1 comprising the amino acid sequence of SEQ ID NO: 1, a HCDR2 comprising the amino acid sequence of SEQ ID NO: 11, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 12;
c) a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 6;
d) a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 12;
e) a HCDR1 comprising the amino acid sequence of SEQ ID NO: 1, a HCDR2 comprising the amino acid sequence of SEQ ID NO: 22, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 12;
f) a VH comprising the amino acid sequence of SEQ ID NO: 7 and a VL comprising the amino acid sequence of SEQ ID NO: 8;
g) a VH comprising the amino acid sequence of SEQ ID NO: 13 and a VL comprising the amino acid sequence of SEQ ID NO: 14;
h) a VH comprising the amino acid sequence of SEQ ID NO: 7 and a VL comprising the amino acid sequence of SEQ ID NO: 18;
i) a VH comprising the amino acid sequence of SEQ ID NO: 7 and a VL comprising the amino acid sequence of SEQ ID NO: 20;
j) a VH comprising the amino acid sequence of SEQ ID NO: 23 and a VL comprising the amino acid sequence of SEQ ID NO: 20;
k) a HC comprising the amino acid sequence of SEQ ID NO: 9 and a LC comprising the amino acid sequence of SEQ ID NO: 10;
l) a HC comprising the amino acid sequence of SEQ ID NO: 15 and a LC comprising the amino acid sequence of SEQ ID NO: 16;
m) a HC comprising the amino acid sequence of SEQ ID NO: 9 and a LC comprising the amino acid sequence of SEQ ID NO: 19;
n) a HC comprising the amino acid sequence of SEQ ID NO: 9 and a LC comprising the amino acid sequence of SEQ ID NO: 21; or
o) a HC comprising the amino acid sequence of SEQ ID NO: 24 and a LC comprising the amino acid sequence of SEQ ID NO: 21.

Disclosed herein are vectors comprising any of the herein disclosed nucleic acid molecules. In some embodiments, the vector comprises a nucleic acid molecule that encodes an antibody or antigen-binding fragment thereof that comprises:
a) a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6;
b) a HCDR1 comprising the amino acid sequence of SEQ ID NO: 1, a HCDR2 comprising the amino acid sequence of SEQ ID NO: 11, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 12;
c) a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 6;
d) a HCDR1 comprising the amino acid sequence of SEQ ID NO: 1, a HCDR2 comprising the amino acid sequence of SEQ ID NO: 2, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 12;
e) a HCDR1 comprising the amino acid sequence of SEQ ID NO: 1, a HCDR2 comprising the amino acid sequence of SEQ ID NO: 22, a HCDR3 comprising the amino acid sequence of SEQ ID NO: 3, a LCDR1 comprising the amino acid sequence of SEQ ID NO: 4, a LCDR2 comprising the amino acid sequence of SEQ ID NO: 17, and a LCDR3 comprising the amino acid sequence of SEQ ID NO: 12;
f) a VH comprising the amino acid sequence of SEQ ID NO: 7 and a VL comprising the amino acid sequence of SEQ ID NO: 8;
g) a VH comprising the amino acid sequence of SEQ ID NO: 13 and a VL comprising the amino acid sequence of SEQ ID NO: 14;
h) a VH comprising the amino acid sequence of SEQ ID NO: 7 and a VL comprising the amino acid sequence of SEQ ID NO: 18;
i) a VH comprising the amino acid sequence of SEQ ID NO: 7 and a VL comprising the amino acid sequence of SEQ ID NO: 20;
j) a VH comprising the amino acid sequence of SEQ ID NO: 23 and a VL comprising the amino acid sequence of SEQ ID NO: 20;
k) a HC comprising the amino acid sequence of SEQ ID NO: 9 and a LC comprising the amino acid sequence of SEQ ID NO: 10;
1) a HC comprising the amino acid sequence of SEQ ID NO: 15 and a LC comprising the amino acid sequence of SEQ ID NO: 16;
m) a HC comprising the amino acid sequence of SEQ ID NO: 9 and a LC comprising the amino acid sequence of SEQ ID NO: 19;
n) a HC comprising the amino acid sequence of SEQ ID NO: 9 and a LC comprising the amino acid sequence of SEQ ID NO: 21; or
o) a HC comprising the amino acid sequence of SEQ ID NO: 24 and a LC comprising the amino acid sequence of SEQ ID NO: 21.

Disclosed herein are cells comprising any of the herein disclosed antibodies or antigen-binding fragments thereof, any of the disclosed nucleic acid molecules, or any of the disclosed vectors.

Disclosed herein are cells expressing any of the herein disclosed antibodies or antigen-binding fragments thereof.

It is to be appreciated that certain features of the disclosed antibodies, nucleic acids, vectors, and cells which are, for clarity, described herein in the context of separate embodiments, may also be provided in combination in a single embodiment. Conversely, various features of the disclosed antibodies, nucleic acids, vectors, and cells that are, for brevity, described in the context of a single embodiment, may also be provided separately or in any subcombination.

In some embodiments, provided herein is an anti-TL1A antibody or an antigen-binding portion thereof, wherein the anti-TL1A antibody or antigen-binding portion binds specifically to human TL1A and (a) inhibits secretion of one or more pro-inflammatory cytokines (e.g., IFNγ, GM-CSF, and/or TNFα), and/or (b) reduces apoptosis of target cells (e.g., TL1A+ cells or DR3+ cells). In some embodiments, provided herein is an anti-IL1β antibody or antigen-binding portion thereof, wherein the anti-IL1β antibody or antigen-binding portion thereof binds specifically to human IL1β and (a) inhibits secretion of one or more pro-inflammatory cytokines (e.g., IL6, TNFα, and/or IL17), and/or (b) reduces pyroptosis of target cells (e.g., IL1β+ cells or NLRP3+ cells). In some embodiments, provided herein is an anti-IL13 antibody or antigen-binding portion thereof, wherein the anti-IL13 antibody or antigen-binding portion thereof binds specifically to human IL13 and (a) inhibits secretion of one or more pro-inflammatory cytokines (e.g., IL4, IL5, and/or IgE), and/or (b) reduces the activation of target cells (e.g., B cells, eosinophils, and/or mast cells). In some embodiments, provided herein is an anti-TSLP antibody or antigen-binding portion thereof, wherein the anti-TSLP antibody or antigen-binding portion thereof binds specifically to human TSLP and (a) inhibits the release of pro-inflammatory cytokines (e.g., CCL17, CCL22, IL4, IL5, IL13, and/or TNFα), (b) inhibits STAT5 activation, and/or (c) reduces the activation of target cells (e.g., mast cells, basophils, and eosinophils) involved in allergic inflammatory responses. In some embodiments, provided herein is an anti-IL15 antibody or antigen-binding portion thereof, wherein the anti-IL15 antibody or antigen-binding portions thereof binds specifically to human IL15 and (a) inhibit the secretion of pro-inflammatory cytokines (e.g. TNFα, IL6, and IL1β, and IFNγ), and/or (b) reduces the proliferation of target cells (e.g., T and/or NK cells) to suppress IL15-mediated over activation of the immune system.

In some embodiments, an antibody or antigen-binding portion provided herein has one or more desirable pharmacokinetic properties. In some embodiments, an anti-TL1A antibody or antigen-binding portion provided herein exhibits one or more improved pharmacokinetic properties compared to an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12). In some embodiments, an anti-TL1A antibody or antigen-binding portion provided herein exhibits one or more improved pharmacokinetic properties compared to an anti-TL1A antibody comprising a VH region of SEQ ID NO: 31 and a VL region of SEQ ID NO: 16.

In some embodiments, an antibody or an antigen-binding portion has increased binding affinity for a human recycling receptor compared to a comparator antibody. For example, in some embodiments, an anti-TL1A antibody or an antigen-binding portion has increased binding affinity for a human recycling receptor compared to that of an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12). In some embodiments, an anti-TL1A antibody or an antigen-binding portion has increased binding affinity for a human recycling receptor compared to that of an anti-TL1A antibody comprising a VH region of SEQ ID NO: 31 and a VL region of SEQ ID NO: 16. In some embodiments, an anti-TL1A antibody or an antigen-binding portion has increased binding affinity for a human neonatal (FcRn) receptor. In some embodiments, the increased affinity is at pH 6.0. In some embodiments, binding affinity of an anti-TL1A antibody or an antigen-binding portion thereof for a human FcRn receptor may be measured by BIACORE® analysis (e.g., by Biacore® 8K using a CM5 chip) and may be represented by K_{D}. In some embodiments, binding affinity of an anti-TL1A antibody or an antigen-binding portion thereof for a human FcγRn receptor may be about 1.59E−07 M at pH 6.0. In some embodiments, binding affinity of an anti-TL1A antibody or an antigen-binding portion thereof for a human FcRn receptor may be about 4.53E−08 M at pH 6.0. In some embodiments, binding affinity of an anti-TL1A antibody or an antigen-binding portion thereof for a human FcRn receptor may be increased by about 5-fold to about 100-fold. In some embodiments, binding affinity of an anti-TL1A antibody or an antigen-binding portion thereof for a human FcRn receptor may be increased by about 10-fold to about 60-fold. In some embodiments, binding affinity of an anti-TL1A antibody or an antigen-binding portion thereof for a human FcRn receptor may be increased by at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 25-fold, at least 30-fold, at least 35-fold, at least 40-fold, at least 45-fold, at least 50-fold, or at least 55-fold.

In some embodiments, an antibody or an antigen-binding portion (e.g., an anti-TL1A antibody or antigen binding portion) has at least one improved pharmacokinetic property compared to a comparator antibody (e.g., RVT-3101). In some embodiments, the comparator antibody is RVT-3101, AVTX-009, APG-777, APG-777×AIO-001, or CALY-002. In some embodiments, the one or more pharmacokinetic properties are determined by predictive software. In some embodiments, the predictive software is PK-Sim and/or MOBI. In some embodiments, the one or more pharmacokinetic properties comprise half-life, Cmax, clearance (CL), volume of distribution (V_{d}), volume of distribution at steady state (Vₛₛ), and/or area under the curve (AUC)_{0-∞} or (AUC)₀₋ₜ.

In some embodiments, the half-life of the antibody or antigen-binding portion provided herein is increased compared to that of a comparator antibody. For example, in some embodiments, the half-life of the anti-TL1A antibody or antigen-binding portion thereof provided herein is increased compared to that of an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12). In some embodiments, the half-life of the anti-TL1A antibody or antigen-binding portion provided herein is increased compared to that of an anti-TL1A antibody comprising a VH region of SEQ ID NO: 31 and a VL region of SEQ ID NO: 16. In some embodiments, the half-life of the antibody or antigen-binding portion provided herein is increased by about 20 to about 150 days. In some embodiments, the half-life of the antibody or antigen-binding portion provided herein is increased by about 50 to about 100 days. In some embodiments, the half-life of the antibody or antigen-binding portion provided herein is increased by about 50 days, about 55 days, about 60 days, about 65 days, about 70 days, about 75 days, about 80 days, about 85 days, about 90 days, about 95 days, or about 100 days. In some embodiments, the half-life of the antibody or antigen-binding portion provided herein is increased by about 50 days. In some embodiments, the half-life of the antibody or antigen-binding portion provided herein is increased by about 100 days.

In some embodiments, an antibody or antigen-binding portion (e.g., an anti-TL1A antibody or antigen binding portion) provided herein has reduced immunogenicity compared to that of a comparator antibody. For example, in some embodiments, an anti-TL1A antibody or antigen-binding portion provided herein has reduced immunogenicity compared to that of an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12). In some embodiments, an anti-TL1A antibody or antigen-binding portion exhibits reduced immunogenicity compared to that of an anti-TL1A antibody comprising a VH region of SEQ ID NO: 31 and a VL region of SEQ ID NO: 16.

In some embodiments, an antibody or antigen-binding portion (e.g., an anti-TL1A antibody or antigen binding portion) provided herein exhibits immunogenicity that is substantially similar to that of a comparator antibody. For example, in some embodiments, an anti-TL1A antibody or antigen-binding portion provided herein has substantially similar immunogenicity compared to that of an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12). In some embodiments, an anti-TL1A antibody or antigen-binding portion exhibits immunogenicity that is substantially similar to that of an anti-TL1A antibody comprising a VH region of SEQ ID NO: 31 and a VL region of SEQ ID NO: 16. As used herein, “substantially similar immunogenicity” refers to an immunogenicity value that is below 200% of that of the comparator antibody.

In some examples, the reduced or substantially similar immunogenicity is predicted in silico or is measured in vitro, in vivo, or ex vivo. In some embodiments, immunogenicity is predicted in silico by AlphaMHC software (WeComput Technology). In some embodiments, the predictive software provides an immunogenicity score.

In some embodiments, the immunogenicity score of the antibody or an antigen-binding portion provided herein is reduced by at least 5%, at least 10%, at least 15%, at least 20%, at least 25%, at least 30%, at least 35%, at least 40%, at least 45%, at least 50%, at least 60%, at least 70%, at least 80%, or at least 90%, compared to a comparator antibody. For example, in some embodiments, the immunogenicity score of an anti-TL1A antibody or antigen-binding portion provided herein is reduced compared to that of an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12). In some embodiments, the immunogenicity score of the anti-TL1A antibody or an antigen-binding portion is increased by no more than 5%, compared to an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12). In some embodiments, the immunogenicity score of the anti-TL1A antibody or an antigen-binding portion is increased by no more than 30%, compared to an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12).

In some embodiments, the immunogenicity score of the antibody or antigen-binding portion provided herein is increased by no more than 5%, no more than 10%, no more than 15%, no more than 20%, no more than 25%, no more than 30%, no more than 35%, no more than 40%, no more than 45%, no more than 50%, no more than 60%, no more than 70%, no more than 80%, no more than 90%, no more than 100%, no more than 120%, no more than 140%, no more than 160%, no more than 180%, or no more than 200%, compared to a comparator antibody. For example, in some embodiments, the immunogenicity score of an anti-TL1A antibody or antigen-binding portion provided herein is increased compared to that of an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12). In some embodiments, the immunogenicity score of the anti-TL1A antibody or an antigen-binding portion is increased by no more than 5%, compared to an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12). In some embodiments, the immunogenicity score of the anti-TL1A antibody or an antigen-binding portion is increased by no more than 30%, compared to an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12).

In some embodiments, the immunogenicity is measured via cytokine release from cells (e.g., whole blood or PBMCs from healthy donors) contacted with the antibody or antigen-binding portion thereof. In some embodiments, immunogenicity by cytokine release from cells is reduced by at least 5%, at least 10%, at least 15%, at least 20%, at least 25%, at least 30%, at least 35%, at least 40%, at least 45%, at least 50%, at least 60%, at least 70%, at least 80%, or at least 90%, compared to an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12). In some embodiments, immunogenicity by cytokine release from cells is increased by no more than 20%, no more than 30%, no more than 40%, no more than 50%, no more than 60%, no more than 70%, no more than 80%, no more than 90%, no more than 100%, no more than 120%, no more than 140%, no more than 160%, no more than 180%, no more than 200%, no more than 250%, or no more than 300%, compared to an anti-TL1A antibody comprising an HCDR1 of YYGIS (SEQ ID NO: 25), HCDR2 of WISTYNGNTHYARMLQG (SEQ ID NO: 30), HCDR3 of ENYYGSGAYRGGMDV (SEQ ID NO: 3), LCDR1 of RASQSVSSYLA (SEQ ID NO: 4), LCDR2 of DASNRAT (SEQ ID NO: 5), and LCDR3 of QQRSNWPWT (SEQ ID NO: 12).

Further provided herein is an immunoconjugate comprising an antibody or an antigen-binding portion thereof disclosed herein, linked to a therapeutic agent. In some embodiments, the therapeutic agent is an immunomodulatory agent or a therapeutic nucleic acid.

Examples of suitable therapeutic agents include, but are not limited to, immunomodulatory agents. Further therapeutic agents include a therapeutic nucleic acid, such as a gene encoding an immunomodulatory agent. These drug descriptors are not mutually exclusive, and thus a therapeutic agent may be described using one or more of the above terms.

### Variant Immunoglobulin FC Regions

Also provided herein are polypeptides comprising variant IgG Fc regions. Exemplary amino acid sequences of Fc regions are provided in Table 9. In some embodiments, the variant IgG Fc region comprises amino acid substitutions at: a) position Q311 and/or M428; and b) position H433 and/or N434; relative to a wild-type IgG Fc region, wherein numbering is according to the EU index as in Kabat. In some embodiments, the wild-type IgG Fc region is a mammalian IgG Fc region. In some embodiments, the wild-type IgG Fc region is a human IgG Fc region. In some embodiments, the variant IgG Fc region comprises amino acid substitutions at: a) position Q311 and/or M428; and b) position H433 and/or N434; relative to a reference IgG Fc region, wherein numbering is according to the EU index as in Kabat. In some embodiments, the reference IgG Fc region comprises the amino acid sequence of SEQ ID NO: 42, 107, 108, 109, or 110. In some embodiments, the reference IgG Fc region comprises the amino acid sequence of SEQ ID NO: 42. In some embodiments, the reference IgG Fc region comprises the amino acid sequence of SEQ ID NO: 107. In some embodiments, the reference IgG Fc region comprises the amino acid sequence of SEQ ID NO: 108. In some embodiments, the reference IgG Fc region comprises the amino acid sequence of SEQ ID NO: 109. In some embodiments, the reference IgG Fc region comprises the amino acid sequence of SEQ ID NO: 110.

In some embodiments, a position of an amino acid residue in a constant region of an immunoglobulin molecule is numbered according to the EU index as in Kabat (Ward et al., 1995 Therap. Immunol. 2:77-94).

In some embodiments, the variant IgG Fc region comprises amino acid substitutions at: a) Q311 and H433; b) M428 and N434; c) Q311 and N434; d) M428 and H433; e) Q311, M428, and H433; f) Q311, M428, and N434; g) Q311, H433, and N434; or h) M428, H433, and N434.

In some embodiments, the variant IgG Fc region comprises amino acid substitutions at Q311 and H433. In some embodiments, the variant IgG Fc region comprises amino acid substitutions at M428 and N434. In some embodiments, the variant IgG Fc region comprises amino acid substitutions at Q311 and N434. In some embodiments, the variant IgG Fc region comprises amino acid substitutions at M428 and H433. In some embodiments, the variant IgG Fc region comprises amino acid substitutions at Q311, M428, and H433. In some embodiments, the variant IgG Fc region comprises amino acid substitutions at Q311, M428, and N434. In some embodiments, the variant IgG Fc region comprises amino acid substitutions at M428, H433, and N434. In some embodiments, the variant IgG Fc region comprises amino acid substitutions at Q311, H433, and N434; or M428, H433, and N434.

In some embodiments, the variant IgG Fc region comprises amino acid substitutions at Q311, M428, H433, and N434.

In some embodiments, the amino acid substitutions comprise: a) Q311R and/or M428L; and b) H433K and/or N434F. In some embodiments, the amino acid substitutions comprise: a) Q311R and H433K; b) M428L and N434F; c) Q311R and N434F; d) M428L and H433K; e) Q311R, M428L, and H433K; f) Q311R, M428L, and N434F; g) Q311R, H433K, and N434F; or h) M428L, H433K, and N434F.

In some embodiments, the amino acid substitutions comprise Q311R and H433K. In some embodiments, the amino acid substitutions comprise M428L and N434F. In some embodiments, the amino acid substitutions comprise Q311R and N434F. In some embodiments, the amino acid substitutions comprise M428L and H433K. In some embodiments, the amino acid substitutions comprise Q311R, M428L, and H433K. In some embodiments, the amino acid substitutions comprise Q311R, M428L, and N434F. In some embodiments, the amino acid substitutions comprise Q311R, H433K, and N434F. In some embodiments, the amino acid substitutions comprise M428L, H433K, and N434F.

In some embodiments, the amino acid substitutions comprise: a) Q311R and/or M428L; and b) H433K and/or N434S. In some embodiments, the amino acid substitutions comprise: a) Q311R and H433K; b) M428L and N434S; c) Q311R and N434S; d) M428L and H433K; e) Q311R, M428L, and H433K; f) Q311R, M428L, and N434S; g) Q311R, H433K, and N434S; or h) M428L, H433K, and N434S.

In some embodiments, the amino acid substitutions comprise Q311R and H433K. In some embodiments, the amino acid substitutions comprise M428L and N434S. In some embodiments, the amino acid substitutions comprise Q311R and N434S. In some embodiments, the amino acid substitutions comprise M428L and H433K. In some embodiments, the amino acid substitutions comprise Q311R, M428L, and H433K. In some embodiments, the amino acid substitutions comprise Q311R, M428L, and N434S. In some embodiments, the amino acid substitutions comprise Q311R, H433K, and N434S. In some embodiments, the amino acid substitutions comprise M428L, H433K, and N434S.

In some embodiments, the amino acid substitutions comprise Q311R, M428L, H433K, and N434F. In some embodiments, the amino acid substitutions comprise M428L and N434S.

In some embodiments, the variant IgG Fc region further comprises one or more amino acid substitutions selected from the group consisting of L234A, L235A, and G237A.

In some embodiments, the variant IgG Fc region is an IgG1, IgG2, IgG3, or IgG4, IgA1 or IgA2 Fc region. In some embodiments, the variant Fc region is an IgG1 Fc region. In some embodiments, the variant Fc region is an IgG4 Fc region.

In some embodiments, the variant IgG Fc region is a mammalian IgG Fc region. human IgG Fc region.

In some embodiments, the polypeptide comprising a variant IgG Fc region exhibits one or more improved pharmacokinetic properties compared to a wild-type IgG Fc region. In some embodiments, the polypeptide comprising a variant IgG Fc region exhibits one or more improved pharmacokinetic properties compared to a reference IgG Fc region. In some embodiments, the reference IgG Fc region comprises the amino acid sequence of SEQ ID NO: 42, 107, 108, 109, or 110. In some embodiments, the one or more pharmacokinetic properties are determined by predictive software. In some embodiments, the predictive software comprises PK-Sim. In some embodiments, the predictive software comprises MOBI. In some embodiments, the one or more pharmacokinetic properties comprise half-life, Cmax, clearance (CL), volume of distribution (V_{d}), volume of distribution at steady state (Vₛₛ), and/or area under the curve (AUC)_{0-∞} or (AUC)₀₋ₜ.

In some embodiments, the polypeptide comprising a variant IgG Fc region has increased affinity for an FcRn receptor, compared to a wild-type IgG Fc region. In some embodiments, the polypeptide comprising a variant IgG Fc region has increased affinity for an FcRn receptor, compared to an IgG having a reference IgG Fc region. In some embodiments, the reference IgG Fc region comprises the amino acid sequence of SEQ ID NO: 42, 107, 108, 109, or 110. In some embodiments, the FcRn receptor is a human FcRn receptor (e.g., UniProt #Q8N166). In some embodiments, the increased affinity is measured at pH 6.0. In some embodiments, binding affinity of a polypeptide comprising a variant IgG Fc region for an FcRn receptor may be measured by BIACORE® analysis (e.g., by Biacore® 8K using a CM5 chip) and may be represented by K_{D}. In some embodiments, binding affinity of a polypeptide comprising a variant IgG Fc region for an FcRn receptor may be about 4.53E−08 M at pH 6.0. In some embodiments, binding affinity of a polypeptide comprising a variant IgG Fc region for an FcRn receptor may be about 1.59E−07 M at pH 6.0. In some embodiments, binding affinity of a polypeptide comprising a variant IgG Fc region for an FcRn receptor may be increased by about 5-fold to about 100-fold. In some embodiments, binding affinity of a polypeptide comprising a variant IgG Fc region for an FcRn receptor may be increased by about 10-fold to about 60-fold. In some embodiments, binding affinity of a polypeptide comprising a variant IgG Fc region for an FcRn receptor may be increased by at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 25-fold, at least 30-fold, at least 35-fold, at least 40-fold, at least 45-fold, at least 50-fold, or at least 55-fold.

In some embodiments, the polypeptide comprising a variant IgG Fc region has an increased half-life compared to the half-life of an IgG having a wild-type IgG Fc region. In some embodiments, the polypeptide comprising a variant IgG Fc region has an increased half-life compared to the half-life of an IgG having a reference Fc region. In some embodiments, the reference IgG Fc region comprises the amino acid sequence of SEQ ID NO: 42, 107, 108, 109, or 110. In some embodiments, the half-life is measured using predictive software (e.g., PK-Sim and/or MOBI). In some embodiments, the half-life of the polypeptide comprising a variant IgG Fc region is increased by about 2-fold to about 30-fold. In some embodiments, the half-life of the polypeptide comprising a variant IgG Fc region is increased by about 5-fold to about 15-fold. In some embodiments, the half-life of the polypeptide comprising a variant IgG Fc region is increased by about 5-fold to about 15-fold. In some embodiments, the half-life of the polypeptide comprising a variant IgG Fc region is increased by about 5-fold, about 6-fold, about 7-fold, about 8-fold, about 9-fold, about 10-fold, about 11-fold, about 12-fold, about 13-fold, about 14-fold, or about 15-fold. In some embodiments, the half-life of the polypeptide comprising a variant IgG Fc region is increased by about 6-fold. In some embodiments, the half-life of the polypeptide comprising a variant IgG Fc region is increased by about 11-fold. In some embodiments, the half-life of the polypeptide comprising a variant IgG Fc region is increased by about 20 to about 150 days. In some embodiments, the half-life of the polypeptide comprising a variant IgG Fc region is increased by about 50 to about 100 days. In some embodiments, the half-life of the polypeptide comprising a variant IgG Fc region is increased by about 50 days, about 55 days, about 60 days, about 65 days, about 70 days, about 75 days, about 80 days, about 85 days, about 90 days, about 95 days, or about 100 days. In some embodiments, the half-life of the polypeptide comprising a variant IgG Fc region is increased by about 50 days. In some embodiments, the half-life of the polypeptide comprising a variant IgG Fc region is increased by about 100 days.

In some embodiments, the variant IgG Fc region comprises the amino acid sequence of SEQ ID NO: 40. In some embodiments, the variant IgG Fc region comprises the amino acid sequence of SEQ ID NO: 41. In some embodiments, the variant IgG Fc region comprises the amino acid sequence of SEQ ID NO: 99. In some embodiments, the variant IgG Fc region comprises the amino acid sequence of SEQ ID NO: 100. In some embodiments, the variant IgG Fc region comprises the amino acid sequence of SEQ ID NO: 101. In some embodiments, the variant IgG Fc region comprises the amino acid sequence of SEQ ID NO: 102. In some embodiments, the variant IgG Fc region comprises the amino acid sequence of SEQ ID NO: 133. In some embodiments, the variant IgG Fc region comprises the amino acid sequence of SEQ ID NO: 134. In some embodiments, the variant IgG Fc region comprises the amino acid sequence of SEQ ID NO: 135. In some embodiments, the variant IgG Fc region comprises the amino acid sequence of SEQ ID NO: 136. In some embodiments, the variant IgG Fc region comprises the amino acid sequence of SEQ ID NO: 137.

In some embodiments, the polypeptide comprises an antibody or antigen-binding fragment thereof. In some embodiments, the polypeptide is an antibody. In some embodiments, the antibody is a human antibody, a humanized antibody, or monoclonal antibody. In some embodiments, the antibody or antigen-binding fragment thereof binds tumor necrosis factor-like ligand 1A (TL1A). In some embodiments, the antibody or antigen-binding fragment thereof binds interleukin-1β (IL1β). In some embodiments, the antibody or antigen-binding fragment thereof binds interleukin-13 (IL13). In some embodiments, the antibody or antigen-binding fragment thereof binds thymic stromal lymphopoietin (TSLP). In some embodiments, the antibody or antigen-binding fragment thereof binds interleukin-15 (IL15).

In some embodiments, the antibody comprises two or more antigen binding regions. In some embodiments, a first binding region binds a first antigen and a second binding region binds a second antigen. In some embodiments, one of the two or more binding regions binds IL13 or TSLP. In some embodiments, the antibody comprises a first binding region that binds IL13 and a second binding region that binds TSLP.

Also provided herein is any polypeptide described herein, for use as a medicament.

Also provided herein is an immunoconjugate comprising a polypeptide described herein and a therapeutic agent, wherein the polypeptide is linked to the therapeutic agent.

### Pharmaceutical Compositions

The antibodies, antigen-binding portions, and polypeptides provided herein (also referred to herein as “active compounds”) can be incorporated into pharmaceutical compositions suitable for administration. Such compositions typically comprise an antibody, antigen-binding portion, or polypeptide described herein (or an immunoconjugate comprising said antibody or portion), and a pharmaceutically acceptable carrier, diluent or excipient. Such materials should be non-toxic and should not interfere with the efficacy of the antibody, antigen-binding fragment thereof, or polypeptide. The precise nature of the carrier or other material will depend on the route of administration, which may be by injection, bolus, infusion, or any other suitable route, as discussed below.

As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions. A pharmaceutically acceptable carrier, diluent or excipient may be a compound or a combination of compounds that does not provoke secondary reactions and that allows, for example, facilitation of the administration of the antibodies, antigen-binding portions, or polypeptides provided herein, an increase in its lifespan and/or in its efficacy in the body or an increase in its solubility in solution.

Provided herein is a pharmaceutical composition comprising (i) an antibody or an antigen-binding portion thereof, wherein the antibody or antigen-binding portion comprises a VH region and a VL region, wherein: (a) the VH region amino acid sequence comprises any combination disclosed herein of an HCDR1, an HCDR2, an HCDR3, and the VL region amino acid sequence comprises any combination disclosed herein of an LCDR1, an LCDR2, and an LCDR3; and (ii) a pharmaceutically acceptable carrier, diluent or excipient. For example, provided herein is a pharmaceutical composition comprising (i) an anti-TL1A antibody or antigen-binding portion thereof, wherein the antibody or antigen-binding portion comprises a VH region and a VL region, wherein: (a) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6; and (ii) a pharmaceutically acceptable carrier, diluent or excipient; (b) the VH region amino acid sequence comprises a HCDR1 comprising the amino acid sequence of SEQ ID NO: 1, a HCDR2 comprising the amino acid sequence of SEQ ID NO: 11 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 12; (c) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 6; (d) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 12; (e) the VH region amino acid sequence comprises a HCDR1 comprising the amino acid sequence of SEQ ID NO: 1, a HCDR2 comprising the amino acid sequence of SEQ ID NO: 22 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 12; (f) the VH region amino acid sequence comprises a HCDR1 comprising the amino acid sequence of SEQ ID NO: 25, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6; or (g) the VH region amino acid sequence comprises a HCDR1 comprising the amino acid sequence of SEQ ID NO: 25, a HCDR2 comprising the amino acid sequence of SEQ ID NO: 2 and a HCDR3 comprising the amino acid sequence of SEQ ID NO: 27; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ5 ID NO: 6; and (ii) a pharmaceutically acceptable carrier, diluent or excipient.

Provided herein is a pharmaceutical composition comprising (i) an anti-TL1A antibody or an antigen-binding portion thereof, wherein the antibody or antigen-binding portion comprises a VH region and a VL region, wherein: (a) the VH region amino acid sequence comprises or consists of SEQ ID NO: 7 and the VL region amino acid sequence comprises or consists of SEQ ID NO: 8; (b) the VH region amino acid sequence comprises or consists of SEQ ID NO: 13 and the VL region amino acid sequence comprises or consists of SEQ ID NO: 14; (c) the VH region amino acid sequence comprises or consists of SEQ ID NO: 7 and the VL region amino acid sequence comprises or consists of SEQ ID NO: 18; (d) the VH region amino acid sequence comprises or consists of SEQ ID NO: 7 and the VL region amino acid sequence comprises or consists of SEQ ID NO: 20; (e) the VH region amino acid sequence comprises or consists of SEQ ID NO: 23 and the VL region amino acid sequence comprises or consists of SEQ ID NO: 20; or (f) the VH region amino acid sequence comprises or consists of SEQ ID NO: 28 and the VL region amino acid sequence comprises or consists of SEQ ID NO: 20; and (ii) a pharmaceutically acceptable carrier, diluent or excipient.

Also provided herein is a pharmaceutical composition comprising (i) a polypeptide comprising a variant IgG Fc region described herein; and (ii) a pharmaceutically acceptable carrier, diluent or excipient. In some embodiments, the pharmaceutical composition comprises a polypeptide comprising a variant IgG Fc region comprising substitutions at a) position Q311 and/or M428; and b) position H433 and/or N434; relative to a wild-type IgG Fc region, wherein numbering is according to the EU index as in Kabat. In some embodiments, the pharmaceutical composition comprises a polypeptide comprising a variant IgG Fc region comprising amino acid substitutions at Q311, M428, H433, and N434. In some embodiments, the pharmaceutical composition comprises a polypeptide comprising a variant IgG Fc region comprising amino acid substitutions: a) Q311R and/or M428L; and b) H433K and/or N434F. In some embodiments, the pharmaceutical composition comprises a polypeptide comprising a variant IgG Fc region comprising amino acid substitutions: a) Q311R and H433K; b) M428L and N434F; c) Q311R and N434F; d) M428L and H433K; e) Q311R, M428L, and H433K; f) Q311R, M428L, and N434F; g) Q311R, H433K, and N434F; or h) M428L, H433K, and N434F. In some embodiments, the pharmaceutical composition comprises a polypeptide comprising a variant IgG Fc region comprising amino acid substitutions Q311R, M428L, H433K, and N434F. In some embodiments, the pharmaceutical composition comprises a polypeptide comprising a variant IgG Fc region comprising amino acid substitutions M428L and N434S.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the antibody, antigen-binding portion thereof, or polypeptide described herein may be provided in a lyophilized form for reconstitution prior to administration. For example, lyophilized antibody molecules may be reconstituted in sterile water and mixed with saline prior to administration to an individual.

The pharmaceutical compositions provided herein can be included in a container, pack, or dispenser together with instructions for administration.

### Nucleic Acid Molecules, Vectors, Host Cells and Methods of Producing Antibodies

Provided herein is a nucleic acid molecule (e.g., an isolated nucleic acid molecule) encoding an amino acid sequence of an antibody or anti-TL1A antigen-binding portion described herein (or an amino acid sequence of a (i) VH region, (ii) a VL region, or (iii) both a VH region and a VL region of an antibody or antigen-binding portion). Further provided herein is a nucleic acid molecule (e.g., an isolated nucleic acid molecule) encoding (i) a heavy chain, (ii) a light chain, or (iii) both a heavy chain and a light chain of an antibody or antigen-binding portion described herein. In some embodiments, a nucleic acid molecule encoding a VH region, a VL region, a heavy chain or a light chain comprises a signal sequence. In some embodiments, a nucleic acid molecule encoding a VH region, a VL region, a heavy chain or a light chain does not comprise a signal sequence.

In some embodiments, a nucleic acid molecule encodes an amino acid sequence of a VH region and a VL region of an antibody or an antigen-binding portion thereof, wherein: (a) the VH region amino acid sequence comprises any combination disclosed herein of an HCDR1, an HCDR2, an HCDR3, and (b) the VL region amino acid sequence comprises any combination disclosed herein of an LCDR1, an LCDR2, and an LCDR3. For example, provided herein a nucleic acid molecule encoding an amino acid sequence of a VH region and a VL region of an anti-TL1A antibody or antigen-binding portion thereof, wherein: (a) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6; and (ii) a pharmaceutically acceptable carrier, diluent or excipient; (b) the VH region amino acid sequence comprises a HCDR1 comprising the amino acid sequence of SEQ ID NO: 1, a HCDR2 comprising the amino acid sequence of SEQ ID NO: 11 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 12; (c) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 6; (d) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 12; (e) the VH region amino acid sequence comprises a HCDR1 comprising the amino acid sequence of SEQ ID NO: 1, a HCDR2 comprising the amino acid sequence of SEQ ID NO: 22 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 12; (f) the VH region amino acid sequence comprises a HCDR1 comprising the amino acid sequence of SEQ ID NO: 25, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6; or (g) the VH region amino acid sequence comprises a HCDR1 comprising the amino acid sequence of SEQ ID NO: 25, a HCDR2 comprising the amino acid sequence of SEQ ID NO: 2 and a HCDR3 comprising the amino acid sequence of SEQ ID NO: 27; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6. In some embodiments, a nucleic acid molecule further encodes a human framework region amino acid sequence.

In some embodiments, a nucleic acid molecule encodes an amino acid sequence of a VH region and a VL region of an anti-TL1A antibody or an antigen-binding portion thereof, wherein (a) the VH region amino acid sequence comprises or consists of SEQ ID NO: 7 and the VL region amino acid sequence comprises or consists of SEQ ID NO: 8; (b) the VH region amino acid sequence comprises or consists of SEQ ID NO: 13 and the VL region amino acid sequence comprises or consists of SEQ ID NO: 14; (c) the VH region amino acid sequence comprises or consists of SEQ ID NO: 7 and the VL region amino acid sequence comprises or consists of SEQ ID NO: 18; (d) the VH region amino acid sequence comprises or consists of SEQ ID NO: 7 and the VL region amino acid sequence comprises or consists of SEQ ID NO: 20; (e) the VH region amino acid sequence comprises or consists of SEQ ID NO: 23 and the VL region amino acid sequence comprises or consists of SEQ ID NO: 20; or (f) the VH region amino acid sequence comprises or consists of SEQ ID NO: 28 and the VL region amino acid sequence comprises or consists of SEQ ID NO: 20.

Also provided herein is nucleic acid molecule encoding a polypeptide comprising a variant IgG Fc region described herein. In some embodiments, the nucleic acid molecule encodes a variant IgG Fc region comprising substitutions at a) position Q311 and/or M428; and b) position H433 and/or N434; relative to a wild-type IgG Fc region, wherein numbering is according to the EU index as in Kabat. In some embodiments, the nucleic acid molecule encodes a variant IgG Fc region comprising amino acid substitutions at Q311, M428, H433, and N434. In some embodiments, the nucleic acid molecule encodes a variant IgG Fc region comprising amino acid substitutions: a) Q311R and/or M428L; and b) H433K and/or N434F. In some embodiments, the nucleic acid molecule encodes a variant IgG Fc region comprising amino acid substitutions: a) Q311R and H433K; b) M428L and N434F; c) Q311R and N434F; d) M428L and H433K; e) Q311R, M428L, and H433K; f) Q311R, M428L, and N434F; g) Q311R, H433K, and N434F; or h) M428L, H433K, and N434F. In some embodiments, the nucleic acid molecule encodes a variant IgG Fc region comprising amino acid substitutions Q311R, M428L, H433K, and N434F. In some embodiments, the nucleic acid molecule encodes a variant IgG Fc region comprising amino acid substitutions M428L and N434S.

Also provided herein is an expression vector comprising a nucleic acid molecule described herein. In certain vectors, a nucleic acid molecule is operatively linked to one or more regulatory sequences suitable for expression of the nucleic acid segment in a host cell. In some cases, an expression vector comprises sequences that mediate replication and comprises one or more selectable markers. As used herein, “vector” means a construct that is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Provided herein is a recombinant host cell comprising an expression vector or a nucleic acid molecule disclosed herein. A “host cell” includes an individual cell, a cell line or cell culture that can be or has been a recipient for vector(s) for incorporation of polynucleotide inserts. Host cells include progeny of a single host cell. The progeny may not necessarily be completely identical (in morphology or in genomic DNA complement) to the original parent cell due to natural, accidental, or deliberate mutation. An expression vector can be transfected into a host cell by standard techniques. Non-limiting examples include electroporation, calcium-phosphate precipitation, DEAE-dextran transfection and the like. In some embodiments, a recombinant host cell comprises a single vector or a single nucleic acid molecule encoding both a VH region and a VL region of an anti-TL1A antibody or an antigen-binding portion thereof. In some embodiments, a recombinant host cell comprises (i) a first vector or a first nucleic acid molecule encoding a VH region of an anti-TL1A antibody or an antigen-binding portion thereof and (ii) a second vector or a second nucleic acid molecule encoding a VL region of an anti-TL1A antibody or an antigen-binding portion thereof. In some embodiments, a recombinant host cell comprises a single vector or a single nucleic acid molecule encoding a polypeptide comprising a variant Fc region described herein.

Antibody molecules of the invention, or antigen-binding portion thereof, or polypeptides comprising a variant Fc region, can be produced using techniques well known in the art, for example, recombinant technologies, phage display technologies, synthetic technologies, computational technologies or combinations of such technologies or other technologies readily known in the art.

Further provided herein is a method for producing an anti-TL1A antibody or an antigen-binding portion thereof, the method comprising: culturing a recombinant host cell comprising an expression vector described herein under conditions whereby the nucleic acid segment is expressed, thereby producing the anti-TL1A antibody or antigen-binding portion. The antibody or antigen-binding portion may then be isolated from the host cell or culture. Anti-TL1A antibodies and antigen-binding portions thereof can be produced by any of a variety of methods known to those skilled in the art. In certain embodiments, anti-TL1A antibodies and antigen-binding portions thereof can be produced recombinantly. For example, nucleic acid sequences encoding one or more of SEQ ID NOs: 1-19, or portions thereof, may be introduced into a bacterial cell (e.g., E. coli, B. subtilis) or a eukaryotic cell (e.g., a yeast such as S. cerevisiae, or a mammalian cell such as a CHO cell line, various Cos cell lines, a HeLa cell, a HEK293 cell, various myeloma cell lines, or a transformed B-cell or hybridoma), or into an in vitro translation system, and the translated polypeptide may be isolated. In some embodiments, antibody light chain proteins and heavy chain proteins are produced in a cell with a signal sequence that is removed upon production of a mature anti-TL1A antibody or antigen-binding portion thereof.

Further provided herein is a method for producing a polypeptide comprising a variant IgG Fc region, the method comprising: culturing a recombinant host cell comprising an expression vector described herein under conditions whereby the nucleic acid segment is expressed, thereby producing the polypeptide comprising a variant IgG Fc region. The polypeptide comprising a variant IgG Fc region may then be isolated from the host cell or culture. Polypeptides comprising a variant IgG Fc region can be produced by any of a variety of methods known to those skilled in the art. In certain embodiments, polypeptides comprising a variant IgG Fc region can be produced recombinantly. For example, nucleic acid sequences comprising a sequence encoding SEQ ID NO: 40, SEQ ID NO: 41, SEQ ID NO: 99, SEQ ID NO: 100, SEQ ID NO: 101, SEQ ID NO: 102, SEQ ID NO: 133, SEQ ID NO: 134, SEQ ID NO: 135, SEQ ID NO: 136, or SEQ ID NO: 137, or portions thereof, may be introduced into a bacterial cell (e.g., E. coli, B. subtilis) or a eukaryotic cell (e.g., a yeast such as S. cerevisiae, or a mammalian cell such as a CHO cell line, various Cos cell lines, a HeLa cell, a HEK293 cell, various myeloma cell lines, or a transformed B-cell or hybridoma), or into an in vitro translation system, and the translated polypeptide may be isolated. In some embodiments, polypeptides comprising a variant IgG Fc region are produced in a cell with a signal sequence that is removed upon production of a mature polypeptide comprising a variant IgG Fc region.

Those skilled in the art will be able to determine whether an antibody or antigen-binding portion comprising a given polypeptide sequence binds to TL1A protein without undue experimentation using standard methodologies, for example, Western blots, ELISA, and the like.

### Uses of Antibodies

Provided herein are methods and uses of the anti-TL1A antibodies, anti-TL1A antigen-binding portions, immunoconjugates and pharmaceutical compositions described herein for providing a therapeutic benefit to a subject with an immune-mediated disease or disorder.

Provided herein is a method for treating or preventing a disease or condition in a subject, comprising administering to the subject a therapeutically effective amount of an antibody, antigen-binding portion, immunoconjugate or pharmaceutical composition disclosed herein. In some embodiments, the antibody or an antigen-binding portion thereof comprises a VH region and a VL region, wherein: (a) the VH region amino acid sequence comprises any combination disclosed herein of a HCDR1, a HCDR2, and an HCDR3; and (b) the VL region amino acid sequence comprises any combination disclosed herein of a LCDR1, a LCDR2, and a LCDR3. For example, in some embodiments, the antibody or antigen-binding portion thereof comprises a VH region and a VL region, wherein (a) the VH region comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6; and (ii) a pharmaceutically acceptable carrier, diluent or excipient; (b) the VH region amino acid sequence comprises a HCDR1 comprising the amino acid sequence of SEQ ID NO: 1, a HCDR2 comprising the amino acid sequence of SEQ ID NO: 11 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 12; (c) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 6; (d) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 12; (e) the VH region amino acid sequence comprises a HCDR1 comprising the amino acid sequence of SEQ ID NO: 1, a HCDR2 comprising the amino acid sequence of SEQ ID NO: 22 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 12; (f) the VH region amino acid sequence comprises a HCDR1 comprising the amino acid sequence of SEQ ID NO: 25, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6; or (g) the VH region amino acid sequence comprises a HCDR1 comprising the amino acid sequence of SEQ ID NO: 25, a HCDR2 comprising the amino acid sequence of SEQ ID NO: 2 and a HCDR3 comprising the amino acid sequence of SEQ ID NO: 27; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6. In some embodiments, the method comprises administering to the subject a therapeutically effective amount of an anti-TL1A antibody or an antigen-binding portion thereof, wherein the antibody or antigen-binding portion comprises a VH region and a VL region, wherein: (a) the VH region amino acid sequence comprises or consists of SEQ ID NO: 7 and the VL region amino acid sequence comprises or consists of SEQ ID NO: 8; (b) the VH region amino acid sequence comprises or consists of SEQ ID NO: 13 and the VL region amino acid sequence comprises or consists of SEQ ID NO: 14; (c) the VH region amino acid sequence comprises or consists of SEQ ID NO: 7 and the VL region amino acid sequence comprises or consists of SEQ ID NO: 18; (d) the VH region amino acid sequence comprises or consists of SEQ ID NO: 7 and the VL region amino acid sequence comprises or consists of SEQ ID NO: 20; (e) the VH region amino acid sequence comprises or consists of SEQ ID NO: 23 and the VL region amino acid sequence comprises or consists of SEQ ID NO: 20; or (f) the VH region amino acid sequence comprises or consists of SEQ ID NO: 28 and the VL region amino acid sequence comprises or consists of SEQ ID NO: 20.

Provided herein is a method for reducing or improving at least one symptom of a disease or condition in a subject, the method comprising administering to the subject a therapeutically effective amount of an antibody, antigen-binding portion, immunoconjugate or pharmaceutical composition disclosed herein. In some embodiments, the antibody or an antigen-binding portion thereof comprises a VH region and a VL region, wherein: (a) the VH region amino acid sequence comprises any combination disclosed herein of a HCDR1, a HCDR2, and an HCDR3; and (b) the VL region amino acid sequence comprises any combination disclosed herein of a LCDR1, a LCDR2, and a LCDR3. For example, in some embodiments, the antibody or antigen-binding portion thereof comprises a VH region and a VL region, wherein (a) the VH region comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6; and (ii) a pharmaceutically acceptable carrier, diluent or excipient; (b) the VH region amino acid sequence comprises a HCDR1 comprising the amino acid sequence of SEQ ID NO: 1, a HCDR2 comprising the amino acid sequence of SEQ ID NO: 11 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 12; (c) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 6; (d) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 12; (e) the VH region amino acid sequence comprises a HCDR1 comprising the amino acid sequence of SEQ ID NO: 1, a HCDR2 comprising the amino acid sequence of SEQ ID NO: 22 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 12; (f) the VH region amino acid sequence comprises a HCDR1 comprising the amino acid sequence of SEQ ID NO: 25, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6; or (g) the VH region amino acid sequence comprises a HCDR1 comprising the amino acid sequence of SEQ ID NO: 25, a HCDR2 comprising the amino acid sequence of SEQ ID NO: 2 and a HCDR3 comprising the amino acid sequence of SEQ ID NO: 27; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6.

In some embodiments, the method comprises administering to the subject a therapeutically effective amount of an antibody or an antigen-binding portion thereof, wherein the antibody or antigen-binding portion comprises a VH region and a VL region, wherein: (a) the VH region amino acid sequence comprises or consists of SEQ ID NO: 7 and the VL region amino acid sequence comprises or consists of SEQ ID NO: 8; (b) the VH region amino acid sequence comprises or consists of SEQ ID NO: 13 and the VL region amino acid sequence comprises or consists of SEQ ID NO: 14; (c) the VH region amino acid sequence comprises or consists of SEQ ID NO: 7 and the VL region amino acid sequence comprises or consists of SEQ ID NO: 18; (d) the VH region amino acid sequence comprises or consists of SEQ ID NO: 7 and the VL region amino acid sequence comprises or consists of SEQ ID NO: 20; (e) the VH region amino acid sequence comprises or consists of SEQ ID NO: 23 and the VL region amino acid sequence comprises or consists of SEQ ID NO: 20; or (f) the VH region amino acid sequence comprises or consists of SEQ ID NO: 28 and the VL region amino acid sequence comprises or consists of SEQ ID NO: 20.

In some embodiments, the method results in improvement of one or more pharmacokinetic properties. In some embodiments, the method results in an improvement in immunogenicity. In some embodiments wherein the method comprises administering to the subject a therapeutically effective amount of an anti-TL1A antibody or an antigen-binding portion thereof, the method results in reduced DR3 signaling. In some embodiments wherein the method comprises administering to the subject a therapeutically effective amount of an anti-IL13 antibody or an antigen-binding portion thereof, the method results in reduced IL4/IL13 signaling. In some embodiments wherein the method comprises administering to the subject a therapeutically effective amount of an anti-IL1β antibody or an antigen-binding portion thereof, the method results in reduced IL6 release and/or reduced pro-inflammatory responses. In some embodiments wherein the method comprises administering to the subject a therapeutically effective amount of an anti-IL15 antibody or an antigen-binding portion thereof, the method results in reduced pro-inflammatory responses. In some embodiments wherein the method comprises administering to the subject a therapeutically effective amount of an anti-TSLP antibody or an antigen-binding portion thereof, the method results in reduced release of inflammatory cytokines. In some embodiments, the disease or condition is associated with increased production of IFNγ, GM-CSF, and/or TNFα.

In some embodiments, the disease or condition is inflammatory bowel disease. In some embodiments, the disease or condition is rheumatoid arthritis, inflammatory bowel disease, psoriasis, primary biliary cirrhosis, systemic lupus erythematosus, and ankylosing spondylitis.

Provided herein is method for reducing DR3 signalling, wherein the method comprises contacting a cell with an anti-TL1A antibody, antigen-binding portion, immunoconjugate or pharmaceutical composition disclosed herein. In some embodiments, the antibody or an antigen-binding portion thereof comprises a VH region and a VL region, wherein: (a) the VH region amino acid sequence comprises any combination disclosed herein of a HCDR1, a HCDR2, and an HCDR3; and (b) the VL region amino acid sequence comprises any combination disclosed herein of a LCDR1, a LCDR2, and a LCDR3. For example, in some embodiments, the antibody or antigen-binding portion thereof comprises a VH region and a VL region, wherein (a) the VH region comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6; (b) the VH region amino acid sequence comprises a HCDR1 comprising the amino acid sequence of SEQ ID NO: 1, a HCDR2 comprising the amino acid sequence of SEQ ID NO: 11 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 12; (c) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 6; (d) the VH region amino acid sequence comprises a HCDR1 comprising the amino acid sequence of SEQ ID NO: 1, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 12; (e) the VH region amino acid sequence comprises a HCDR1 comprising the amino acid sequence of SEQ ID NO: 1, a HCDR2 comprising the amino acid sequence of SEQ ID NO: 22 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 17 and a LCDR3 comprising the amino acid sequence of SEQ ID NO: 12; (f) the VH region amino acid sequence comprises a HCDR1 comprising the amino acid sequence of SEQ ID NO: 25, a HCDR2 comprising the amino acid sequence of SEQ ID NO: 2 and a HCDR3 comprising the amino acid sequence of SEQ ID NO: 3; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6; or (g) the VH region amino acid sequence comprises a HCDR1 comprising the amino acid sequence of SEQ ID NO: 25, a HCDR2 comprising the amino acid sequence of SEQ ID NO: 2 and a HCDR3 comprising the amino acid sequence of SEQ ID NO: 27; and the VL region amino acid sequence comprises a LCDR1 comprising the amino acid sequence of SEQ ID NO: 4, a LCDR2 comprising the amino acid sequence of SEQ ID NO: 5 and a LCDR3 comprising the amino acid sequence of SEQ ID NO: 6. In some embodiments, the method comprises administering to the subject a therapeutically effective amount of an anti-TL1A antibody or an antigen-binding portion thereof, wherein the antibody or antigen-binding portion comprises a VH region and a VL region, wherein: (a) the VH region amino acid sequence comprises or consists of SEQ ID NO: 7 and the VL region amino acid sequence comprises or consists of SEQ ID NO: 8; (b) the VH region amino acid sequence comprises or consists of SEQ ID NO: 13 and the VL region amino acid sequence comprises or consists of SEQ ID NO: 14; (c) the VH region amino acid sequence comprises or consists of SEQ ID NO: 7 and the VL region amino acid sequence comprises or consists of SEQ ID NO: 18; (d) the VH region amino acid sequence comprises or consists of SEQ ID NO: 7 and the VL region amino acid sequence comprises or consists of SEQ ID NO: 20; (e) the VH region amino acid sequence comprises or consists of SEQ ID NO: 23 and the VL region amino acid sequence comprises or consists of SEQ ID NO: 20; or (f) the VH region amino acid sequence comprises or consists of SEQ ID NO: 28 and the VL region amino acid sequence comprises or consists of SEQ ID NO: 20.

Also provided herein is method for reducing IL4/IL13 signalling, wherein the method comprises contacting a cell with an anti-IL13 antibody, antigen-binding portion, immunoconjugate or pharmaceutical composition disclosed herein. In some embodiments, the anti-IL13 antibody or an antigen-binding portion thereof comprises a VH region and a VL region, wherein: (a) the VH region amino acid sequence comprises any combination disclosed herein of a HCDR1, a HCDR2, and an HCDR3; and the VL region amino acid sequence comprises any combination disclosed herein of a LCDR1, a LCDR2, and a LCDR3. For example, in some embodiments, the anti-IL13 antibody or antigen-binding portion thereof comprises a VH region and a VL region, wherein (a) the VH region comprises a HCDR1 comprising the amino acid sequence of SEQ ID NO: 53, a HCDR2 comprising the amino acid sequence of SEQ ID NO: 54 and a HCDR3 comprising the amino acid sequence of SEQ ID NO: 55; and the VL region amino acid sequence comprises a LCDR1 comprising the amino acid sequence of SEQ ID NO: 56, a LCDR2 comprising the amino acid sequence of SEQ ID NO: 57 and a LCDR3 comprising the amino acid sequence of SEQ ID NO: 58; or (b) the VH region comprises a HCDR1 comprising the amino acid sequence of SEQ ID NO: 53, a HCDR2 comprising the amino acid sequence of SEQ ID NO: 54 and a HCDR3 comprising the amino acid sequence of SEQ ID NO: 82; and the VL region amino acid sequence comprises a LCDR1 comprising the amino acid sequence of SEQ ID NO: 56, a LCDR2 comprising the amino acid sequence of SEQ ID NO: 57 and a LCDR3 comprising the amino acid sequence of SEQ ID NO: 58. In some embodiments, the anti-IL13 antibody or an antigen-binding portion thereof binds specifically to human IL13. In some embodiments, the anti-IL13 antibody or an antigen-binding portion thereof inhibits secretion of one or more cytokines (e.g., IL4 or IL5). In some embodiments, at least one of the one or more cytokines is a pro-inflammatory cytokine. In some embodiments, the anti-IL13 antibody or an antigen-binding portion thereof inhibits secretion of IgE. In some embodiments, the anti-IL13 antibody or an antigen-binding portion thereof reduces activation of target cells (e.g., B cells, eosinophils, and/or mast cells).

Also provided herein is method for reducing IL6 release, wherein the method comprises contacting a cell with an anti-IL1β antibody, antigen-binding portion, immunoconjugate or pharmaceutical composition disclosed herein. In some embodiments, the anti-IL1β antibody or an antigen-binding portion thereof comprises a VH region and a VL region, wherein the VH region amino acid sequence comprises any combination disclosed herein of a HCDR1, a HCDR2, and an HCDR3; and the VL region amino acid sequence comprises any combination disclosed herein of a LCDR1, a LCDR2, and a LCDR3. For example, in some embodiments, the anti-IL1β antibody or antigen-binding portion thereof comprises a VH region and a VL region, wherein (a) the VH region comprises a HCDR1 comprising the amino acid sequence of SEQ ID NO: 43, a HCDR2 comprising the amino acid sequence of SEQ ID NO: 44 and a HCDR3 comprising the amino acid sequence of SEQ ID NO: 45; and the VL region amino acid sequence comprises a LCDR1 comprising the amino acid sequence of SEQ ID NO: 46, a LCDR2 comprising the amino acid sequence of SEQ ID NO: 47 and a LCDR3 comprising the amino acid sequence of SEQ ID NO: 48. In some embodiments, the anti-IL1β antibody or an antigen-binding portion thereof binds specifically to human IL1β. In some embodiments, the anti-IL1β antibody or an antigen-binding portion thereof inhibits secretion of one or more pro-inflammatory cytokines (e.g., IL6, TNFα, and/or IL17). In some embodiments, the anti-IL1β antibody or an antigen-binding portion thereof reduces pyroptosis of target cells (e.g., IL1β+ cells or NLRP3+ cells).

Also provided herein is method for reducing inflammatory responses, wherein the method comprises contacting a cell with an anti-IL15 antibody, antigen-binding portion, immunoconjugate or pharmaceutical composition disclosed herein. In some embodiments, the anti-IL15 antibody or an antigen-binding portion thereof comprises a VH region and a VL region, wherein the VH region amino acid sequence comprises any combination disclosed herein of a HCDR1, a HCDR2, and an HCDR3; and the VL region amino acid sequence comprises any combination disclosed herein of a LCDR1, a LCDR2, and a LCDR3. For example, in some embodiments, the anti-IL15 antibody or antigen-binding portion thereof comprises a VH region and a VL region, wherein (a) the VH region comprises a HCDR1 comprising the amino acid sequence of SEQ ID NO: 66, a HCDR2 comprising the amino acid sequence of SEQ ID NO: 67 and a HCDR3 comprising the amino acid sequence of SEQ ID NO: 68; and the VL region amino acid sequence comprises a LCDR1 comprising the amino acid sequence of SEQ ID NO: 69, a LCDR2 comprising the amino acid sequence of SEQ ID NO: 70 and a LCDR3 comprising the amino acid sequence of SEQ ID NO: 71. In some embodiments, the anti-IL15 antibody or an antigen-binding portion thereof binds specifically to human IL15. In some embodiments, the anti-IL15 antibody or an antigen-binding portion thereof inhibits release of one or more cytokines (e.g., TNFα, IL6, IL1β, and/or IFNγ). In some embodiments, at least one of the one or more cytokines is a pro-inflammatory cytokine. In some embodiments, the anti-IL15 antibody or an antigen-binding portion thereof reduces proliferation of target cells (e.g., T cells and/or NK cells). In some embodiments, the target cells are cells known to be involved in IL15-mediated activation of the immune system.

Also provided herein is method for reducing release of inflammatory cytokines, wherein the method comprises contacting a cell with an anti-TSLP antibody, antigen-binding portion, immunoconjugate or pharmaceutical composition disclosed herein. In some embodiments, the anti-TSLP antibody or an antigen-binding portion thereof comprises a VH region and a VL region, wherein the VH region amino acid sequence comprises any combination disclosed herein of a HCDR1, a HCDR2, and an HCDR3; and the VL region amino acid sequence comprises any combination disclosed herein of a LCDR1, a LCDR2, and a LCDR3. For example, in some embodiments, the anti-TSLP antibody or antigen-binding portion thereof comprises a VH region and a VL region, wherein (a) the VH region comprises a HCDR1 comprising the amino acid sequence of SEQ ID NO: 76, a HCDR2 comprising the amino acid sequence of SEQ ID NO: 77 and a HCDR3 comprising the amino acid sequence of SEQ ID NO: 78; and the VL region amino acid sequence comprises a LCDR1 comprising the amino acid sequence of SEQ ID NO: 79, a LCDR2 comprising the amino acid sequence of SEQ ID NO: 80 and a LCDR3 comprising the amino acid sequence of SEQ ID NO: 81. In some embodiments, the anti-TSLP antibody or an antigen-binding portion thereof inhibits release of one or more cytokines (e.g., CCL17, CCL22, IL4, IL5, IL13, and/or TNFα). In some embodiments, at least one of the one or more cytokines is a pro-inflammatory cytokine. In some embodiments, the anti-TSLP antibody or an antigen-binding portion thereof reduces activation of target cells (e.g., mast cells, basophils, and/or eosinophils). In some embodiments, the target cells are cells known to be involved in allergic inflammatory responses.

In some embodiments, the method results in reduced proliferation of the cell. In some embodiments, the method results in reduced production of a cytokine. In some embodiments, the cytokine is IFNγ, GM-CSF, TNFα, IL6, IL17, IL4, IL5, CCL17, CCL22, IL13, and/or IL1β. In some embodiments, the cytokine is IL6, TNFα, and/or IL17. In some embodiments, the cytokine is IL4 and/or IL5. In some embodiments, the cytokine is CCL17, CCL22, IL4, IL5, IL13, and/or TNFα. In some embodiments, the cytokine is TNFα, IL6, and IL1β, and/or IFNγ. In some embodiments, the cytokine is IFNγ, GM-CSF, and/or TNFα. In some embodiments, the method results in reduced apoptotic cell death of the cell. In some embodiments, the method results in reduced activation of target cells.

As used herein, the term “effective amount” or “therapeutically effective amount” refers to the amount of a pharmaceutical agent, e.g., an antibody or an antigen-binding portion thereof, which is sufficient to reduce or ameliorate the severity and/or duration of a disease, e.g., inflammatory bowel disease, or one or more symptoms thereof, prevent the advancement of a disease, cause regression of a disease, prevent the recurrence, development, onset or progression of one or more symptoms associated with a disease, detect a disease, or enhance or improve the prophylactic or therapeutic effect(s) of another related therapy (e.g., prophylactic or therapeutic agent) for a disease (e.g., a TL1A-mediated disease).

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of general practitioners and other medical doctors and may depend on the severity of the symptoms and/or progression of a disease being treated. Appropriate doses of antibody molecules are known in the art (Ledermann J. A. et al., 1991, Int. J. Cancer 47: 659-664; Bagshawe K. D. et al., 1991, Antibody, Immunoconjugates and Radiopharmaceuticals 4: 915-922). Specific dosages may be indicated herein or in the Physician's Desk Reference (2003) as appropriate for the type of medicament being administered may be used. A therapeutically effective amount or suitable dose of an antibody molecule may be determined by comparing its in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g., whole antibody, fragment) and the nature of any detectable label or other molecule attached to the antibody.

In some embodiments, antibody molecules and antigen-binding portions as described herein may be administered as sub-cutaneous injections. Sub-cutaneous injections may be administered using an auto-injector, for example for long term prophylaxis/treatment.

In some embodiments, the therapeutic effect of an antibody or an antigen-binding portion thereof may persist for several half-lives, depending on the dose. For example, the therapeutic effect of a single dose of an antibody or an antigen-binding portion thereof may persist in a subject for 2 months or more, 3 months or more, 4 months or more, 5 months or more, 6 months or more, 7 months or more, 8 months or more, 9 months or more, 10 months or more, 11 months or more, or 12 months or more.

In some embodiments, a subject may be treated with an antibody or an antigen-binding portion, an immunoconjugate or a pharmaceutical composition described herein and an additional therapeutic agent or therapy that is used to treat a disease or disorder or a symptom or complication of a disease or disorder. The antibody or an antigen-binding portion and the additional therapeutic agent or therapy may be administered simultaneously or sequentially.

In some embodiments, a subject is a human, a non-human primate, a pig, a horse, a cow, a dog, a cat, a guinea pig, a mouse or a rat. In some embodiments, a subject is an adult human. In some embodiments, a subject is a pediatric human.

Further provided herein is an antibody or an antigen-binding portion, an immunoconjugate or a pharmaceutical composition described herein, for use in the treatment of a disease or a disorder.

Provided herein is an antibody or an antigen-binding portion, an immunoconjugate or a pharmaceutical composition described herein, for use as a medicament.

### Definitions

Unless otherwise noted, the terms used herein have definitions as ordinarily used in the art. Some terms are defined below, and additional definitions can be found within the rest of the detailed description.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 Oct. 11) Molecular systems biology 7:539. For the purposes of calculating identity to the sequence, extensions, such as tags, are not included.

As used herein, the term “HCDR” refers to a heavy chain complementarity determining region. As used herein, the term “LCDR” refers to a light chain complementarity determining region.

As used herein, the term “conservative substitution” refers to replacement of an amino acid with another amino acid which does not significantly deleteriously change the functional activity. A preferred example of a “conservative substitution” is the replacement of one amino acid with another amino acid which has a value ≥0 in the following BLOSUM 62 substitution matrix (see Henikoff & Henikoff, 1992, PNAS 89: 10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4. |

“Antibody-drug conjugate” and “immunoconjugate” refer to an antibody molecule, or antigen-binding portion thereof, including antibody derivatives, that binds to TL1A and is conjugated to cytotoxic, cytostatic and/or therapeutic agents.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The term “epitope” refers to that portion of a molecule capable of being recognized by and bound by an antibody molecule, or antigen-binding portion thereof, at one or more of the antibody molecule's antigen-binding regions. Epitopes can consist of defined regions of primary secondary or tertiary protein structure and includes combinations of secondary structural units or structural domains of the target recognized by the antigen binding regions of the antibody, or antigen-binding portion thereof. Epitopes can likewise consist of a defined chemically active surface grouping of molecules such as amino acids or sugar side chains and have specific three-dimensional structural characteristics as well as specific charge characteristics. The term “antigenic epitope” as used herein, is defined as a portion of a polypeptide to which an antibody molecule can specifically bind as determined by any method well known in the art, for example, by conventional immunoassays, antibody competitive binding assays or by x-ray crystallography or related structural determination methods (for example, nuclear magnetic resonance spectroscopy).

The term “potency” is a measurement of biological activity and may be designated as IC₅₀, EC₅₀, or effective concentration of an antibody or antibody drug conjugate to the antigen TL1A to inhibit 50% of activity measured in a TL1A activity assay as described herein.

The term “inhibit” or “neutralize” as used herein with respect to bioactivity of an antibody disclosed herein means the ability of the antibody to substantially antagonize, prohibit, prevent, restrain, slow, disrupt, eliminate, stop, reduce or reverse for example progression or severity of that which is being inhibited including, but not limited to, a biological activity or binding interaction of the antibody molecule to TL1A.

In the present description, any concentration range, percentage range, ratio range, or integer range is to be understood to include the value of any integer within the recited range and, when appropriate, fractions thereof (such as one tenth and one hundredth of an integer), unless otherwise indicated. The use of the alternative (e.g., “or”) should be understood to mean either one, both, or any combination thereof of the alternatives. As used herein, the terms “include” and “comprise” are used synonymously.

The term “comprising” is intended to include examples encompassed by the terms “consisting essentially of” and “consisting of;” similarly, the term “consisting essentially of” is intended to include examples encompassed by the term “consisting of.”

The phrase “immunospecifically binds” refers to the ability of the disclosed antibodies and antigen-binding fragments thereof to preferentially bind to TL1A without preferentially binding other molecules. Antibodies and antigen-binding fragments thereof that immunospecifically bind TL1A are substantially free of other antibodies or antigen-binding fragments having different antigenic specificities. Antibodies or antigen-binding fragments thereof that immunospecifically bind TL1A, however, can have cross-reactivity to other antigens, such as orthologs of human TL1A. The antibodies and antigen-binding fragments thereof are able to immunospecifically bind both naturally-produced human TL1A and to TL1A which is recombinantly produced in mammalian or prokaryotic cells.

“Antigen-binding fragments” refer to a portion of the TL1A antibodies that retain the antigen binding properties of the parental full length TL1A antibody. Exemplary antigen-binding fragments can comprise heavy chain complementarity determining regions (HCDR) 1, 2, and 3, and light chain complementarity determining regions (LCDR) 1, 2, and 3. Antigen-binding fragments can include, for example, a Fab fragment, a F(ab)₂ fragment, a Fv fragment, a single chain Fv (scFv), and a diabody. Antigen-binding fragments can be obtained using techniques well known to those of skill in the art, and the fragments can be screened for utility in the same manner as are full length antibodies.

The section headings used herein are for organizational purposes only and are not to be construed as limiting the subject matter described.

All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

Any of the aspects and embodiments described herein can be combined with any other aspect or embodiment as disclosed here in the Summary, in the Drawings, and/or in the Detailed Description, including the below specific, non-limiting, examples/embodiments of the present disclosure.

TABLE 1
Annotated amino acid sequences of anti-TLIA Antibodies

| SEQ | Name (of antibody | |
|---|---|---|
| ID NO | and domain or region) | Sequence |
| 1 | AB_TL1A_02_004 | GYDFTYYGIS |
| | HCDR1 | |
| 2 | AB_TL1A_02_004 | WISPYNGNTHYAQKLQG |
| | HCDR2 | |
| 3 | AB_TL1A_02_004 | ENYYGSGAYRGGMDV |
| | HCDR3 | |
| 4 | AB_TL1A_02_004 | RASQSVSSYLA |
| | LCDR1 | |
| 5 | AB_TL1A_02_004 | DASNRAT |
| | LCDR2 | |
| 6 | AB_TL1A_02_004 | QQRSSWPWT |
| | LCDR3 | |
| 7 | AB_TL1A_02_004 | QVQLVQSGAEVKKPGASVKVSCKASGYDFTYYGISWVRQ |
| | VH | APGQGLEWMGWISPYNGNTHYAQKLQGRVTMTTDTSTRT |
| | | AYMELRSLRSDDTAVYYCARENYYGSGAYRGGMDVWGQ |
| | | GTTVTVSS |
| 8 | AB_TL1A_02_004 | EIVLTQSPATLSLSPGERATLSCRASQSVSSYLAWYQQKPG |
| | VL | QAPRLLIYDASNRATGIPARFSGSGSGTDFTLTISSLEPEDFA |
| | | VYYCQQRSSWPWTFGQGTKVEIK |
| 9 | AB_TL1A_02_004 | QVQLVQSGAEVKKPGASVKVSCKASGYDFTYYGISWVRQ |
| | HC | APGQGLEWMGWISPYNGNTHYAQKLQGRVTMTTDTSTRT |
| | | AYMELRSLRSDDTAVYYCARENYYGSGAYRGGMDVWGQ |
| | | GTTVTVSSASTKGPSVFPLAPSSKSTSGGTAALGCLVKDYF |
| | | PEPVTVSWNSGALTSGVHTFPAVLQSSGLYSLSSVVTVPSS |
| | | SLGTQTYICNVNHKPSNTKVDKKVEPKSCDKTHTCPPCPAP |
| | | EAAGAPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPP |
| | | SREEMTKNQVSLTCLVKGFYPSDIAVEWESNGQPENNYKT |
| | | TPPVLDSDGSFFLYSKLTVDKSRWQQGNVFSCSVMHEALH |
| | | NHYTQKSLSLSPG |
| 10 | AB_TL1A_02_004 | EIVLTQSPATLSLSPGERATLSCRASQSVSSYLAWYQQKPG |
| | LC | QAPRLLIYDASNRATGIPARFSGSGSGTDFTLTISSLEPEDFA |
| | | VYYCQQRSSWPWTFGQGTKVEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 1 | AB_TL1A_01_005 | GYDFTYYGIS |
| | HCDR1 | |
| 11 | AB_TL1A_01_005 | WISPYNGNTHYARMLQG |
| | HCDR2 | |
| 3 | AB_TL1A_01_005 | ENYYGSGAYRGGMDV |
| | HCDR3 | |
| 4 | AB_TL1A_01_005 | RASQSVSSYLA |
| | LCDR1 | |
| 5 | AB_TL1A_01_005 | DASNRAT |
| | LCDR2 | |
| 12 | AB_TL1A_01_005 | QQRSNWPWT |
| | LCDR3 | |
| 13 | AB_TL1A_01_005 VH | QVQLVQSGAEVKKPGASVKVSCKASGYDFTYYGISWVRQ |
| | | APGQGLEWMGWISPYNGNTHYARMLQGRVTMTTDTSTRT |
| | | AYMELRSLRSDDTAVYYCARENYYGSGAYRGGMDVWGQ |
| | | GTTVTVSS |
| 14 | AB_TL1A_01_005 VL | EIVLTQSPATLSLSPGERATLSCRASQSVSSYLAWYQQKPG |
| | | QAPRLLIYDASNRATGIPARFSGSGSGTDFTLTISSLEPEDFA |
| | | VYYCQQRSNWPWTFGQGTKVEIK |
| 15 | AB_TL1A_01_005 HC | QVQLVQSGAEVKKPGASVKVSCKASGYDFTYYGISWVRQ |
| | | APGQGLEWMGWISPYNGNTHYARMLQGRVTMTTDTSTRT |
| | | AYMELRSLRSDDTAVYYCARENYYGSGAYRGGMDVWGQ |
| | | GTTVTVSSASTKGPSVFPLAPSSKSTSGGTAALGCLVKDYF |
| | | PEPVTVSWNSGALTSGVHTFPAVLQSSGLYSLSSVVTVPSS |
| | | SLGTQTYICNVNHKPSNTKVDKKVEPKSCDKTHTCPPCPAP |
| | | EAAGAPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPP |
| | | SREEMTKNQVSLTCLVKGFYPSDIAVEWESNGQPENNYKT |
| | | TPPVLDSDGSFFLYSKLTVDKSRWQQGNVFSCSVMHEALH |
| | | NHYTQKSLSLSPG |
| 16 | AB_TL1A_01_005 LC | EIVLTQSPATLSLSPGERATLSCRASQSVSSYLAWYQQKPG |
| | | QAPRLLIYDASNRATGIPARFSGSGSGTDFTLTISSLEPEDFA |
| | | VYYCQQRSNWPWTFGQGTKVEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 1 | AB_TL1A_02_002 | GYDFTYYGIS |
| | HCDR1 | |
| 2 | AB_TL1A_02_002 | WISPYNGNTHYAQKLQG |
| | HCDR2 | |
| 3 | AB_TL1A_02_002 | ENYYGSGAYRGGMDV |
| | HCDR3 | |
| 4 | AB_TL1A_02_002 | RASQSVSSYLA |
| | LCDR1 | |
| 17 | AB_TL1A_02_002 | DASTRAT |
| | LCDR2 | |
| 6 | AB_TL1A_02_002 | QQRSSWPWT |
| | LCDR3 | |
| 7 | AB_TL1A_02_002 VH | QVQLVQSGAEVKKPGASVKVSCKASGYDFTYYGISWVRQ |
| | | APGQGLEWMGWISPYNGNTHYAQKLQGRVTMTTDTSTRT |
| | | AYMELRSLRSDDTAVYYCARENYYGSGAYRGGMDVWGQ |
| | | GTTVTVSS |
| 18 | AB_TL1A_02_002 VL | EIVLTQSPATLSLSPGERATLSCRASQSVSSYLAWYQQKPG |
| | | QAPRLLIYDASTRATGIPARFSGSGSGTDFTLTISSLEPEDFA |
| | | VYYCQQRSSWPWTFGQGTKVEIK |
| 9 | AB_TL1A_02_002 HC | QVQLVQSGAEVKKPGASVKVSCKASGYDFTYYGISWVRQ |
| | | APGQGLEWMGWISPYNGNTHYAQKLQGRVTMTTDTSTRT |
| | | AYMELRSLRSDDTAVYYCARENYYGSGAYRGGMDVWGQ |
| | | GTTVTVSSASTKGPSVFPLAPSSKSTSGGTAALGCLVKDYF |
| | | PEPVTVSWNSGALTSGVHTFPAVLQSSGLYSLSSVVTVPSS |
| | | SLGTQTYICNVNHKPSNTKVDKKVEPKSCDKTHTCPPCPAP |
| | | EAAGAPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPP |
| | | SREEMTKNQVSLTCLVKGFYPSDIAVEWESNGQPENNYKT |
| | | TPPVLDSDGSFFLYSKLTVDKSRWQQGNVFSCSVMHEALH |
| | | NHYTQKSLSLSPG |
| 19 | AB_TL1A_02_002 LC | EIVLTQSPATLSLSPGERATLSCRASQSVSSYLAWYQQKPG |
| | | QAPRLLIYDASTRATGIPARFSGSGSGTDFTLTISSLEPEDFA |
| | | VYYCQQRSSWPWTFGQGTKVEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 1 | AB_TL1A_02_005 | GYDFTYYGIS |
| | HCDR1 | |
| 2 | AB_TL1A_02_005 | WISPYNGNTHYAQKLQG |
| | HCDR2 | |
| 3 | AB_TL1A_02_005 | ENYYGSGAYRGGMDV |
| | HCDR3 | |
| 4 | AB_TL1A_02_005 | RASQSVSSYLA |
| | LCDR1 | |
| 17 | AB_TL1A_02_005 | DASTRAT |
| | LCDR2 | |
| 12 | AB_TL1A_02_005 | QQRSNWPWT |
| | LCDR3 | |
| 7 | AB_TL1A_02_005 VH | QVQLVQSGAEVKKPGASVKVSCKASGYDFTYYGISWVRQ |
| | | APGQGLEWMGWISPYNGNTHYAQKLQGRVTMTTDTSTRT |
| | | AYMELRSLRSDDTAVYYCARENYYGSGAYRGGMDVWGQ |
| | | GTTVTVSS |
| 20 | AB_TL1A_02_005 VL | EIVLTQSPATLSLSPGERATLSCRASQSVSSYLAWYQQKPG |
| | | QAPRLLIYDASTRATGIPARFSGSGSGTDFTLTISSLEPEDFA |
| | | VYYCQQRSNWPWTFGQGTKVEIK |
| 9 | AB_TL1A_02_005 HC | QVQLVQSGAEVKKPGASVKVSCKASGYDFTYYGISWVRQ |
| | | APGQGLEWMGWISPYNGNTHYAQKLQGRVTMTTDTSTRT |
| | | AYMELRSLRSDDTAVYYCARENYYGSGAYRGGMDVWGQ |
| | | GTTVTVSSASTKGPSVFPLAPSSKSTSGGTAALGCLVKDYF |
| | | PEPVTVSWNSGALTSGVHTFPAVLQSSGLYSLSSVVTVPSS |
| | | SLGTQTYICNVNHKPSNTKVDKKVEPKSCDKTHTCPPCPAP |
| | | EAAGAPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPP |
| | | SREEMTKNQVSLTCLVKGFYPSDIAVEWESNGQPENNYKT |
| | | TPPVLDSDGSFFLYSKLTVDKSRWQQGNVFSCSVMHEALH |
| | | NHYTQKSLSLSPG |
| 21 | AB_TL1A_02_005 LC | EIVLTQSPATLSLSPGERATLSCRASQSVSSYLAWYQQKPG |
| | | QAPRLLIYDASTRATGIPARFSGSGSGTDFTLTISSLEPEDFA |
| | | VYYCQQRSNWPWTFGQGTKVEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 1 | AB_TL1A_02_009 | GYDFTYYGIS |
| | HCDR1 | |
| 22 | AB_TL1A_02_009 | WISPYNGNTHYAQMLQG |
| | HCDR2 | |
| 3 | AB_TL1A_02_009 | ENYYGSGAYRGGMDV |
| | HCDR3 | |
| 4 | AB_TL1A_02_009 | RASQSVSSYLA |
| | LCDR1 | |
| 17 | AB_TL1A_02_009 | DASTRAT |
| | LCDR2 | |
| 12 | AB_TL1A_02_009 | QQRSNWPWT |
| | LCDR3 | |
| 23 | AB_TL1A_02_009 VH | QVQLVQSGAEVKKPGASVKVSCKASGYDFTYYGISWVRQ |
| | | APGQGLEWMGWISPYNGNTHYAQMLQGRVTMTTDTSTRT |
| | | AYMELRSLRSDDTAVYYCARENYYGSGAYRGGMDVWGQ |
| | | GTTVTVSS |
| 20 | AB_TL1A_02_009 VL | EIVLTQSPATLSLSPGERATLSCRASQSVSSYLAWYQQKPG |
| | | QAPRLLIYDASTRATGIPARFSGSGSGTDFTLTISSLEPEDFA |
| | | VYYCQQRSNWPWTFGQGTKVEIK |
| 24 | AB_TL1A_02_009 HC | QVQLVQSGAEVKKPGASVKVSCKASGYDFTYYGISWVRQ |
| | | APGQGLEWMGWISPYNGNTHYAQMLQGRVTMTTDTSTRT |
| | | AYMELRSLRSDDTAVYYCARENYYGSGAYRGGMDVWGQ |
| | | GTTVTVSSASTKGPSVFPLAPSSKSTSGGTAALGCLVKDYF |
| | | PEPVTVSWNSGALTSGVHTFPAVLQSSGLYSLSSVVTVPSS |
| | | SLGTQTYICNVNHKPSNTKVDKKVEPKSCDKTHTCPPCPAP |
| | | EAAGAPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPP |
| | | SREEMTKNQVSLTCLVKGFYPSDIAVEWESNGQPENNYKT |
| | | TPPVLDSDGSFFLYSKLTVDKSRWQQGNVFSCSVMHEALH |
| | | NHYTQKSLSLSPG |
| 21 | AB_TL1A_02_009 LC | EIVLTQSPATLSLSPGERATLSCRASQSVSSYLAWYQQKPG |
| | | QAPRLLIYDASTRATGIPARFSGSGSGTDFTLTISSLEPEDFA |
| | | VYYCQQRSNWPWTFGQGTKVEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 25 | AB_TL1A_HL2_HCD | YYGIS |
| | R1 | |
| 2 | AB_TL1A_HL2_HCD | WISPYNGNTHYAQKLQG |
| | R2 | |
| 3 | AB_TL1A_HL2_HCD | ENYYGSGAYRGGMDV |
| | R3 | |
| 4 | AB_TL1A_HL2_LCD | RASQSVSSYLA |
| | R1 | |
| 5 | AB_TL1A_HL2_LCD | DASNRAT |
| | R2 | |
| 6 | AB_TL1A_HL2_LCD | QQRSSWPWT |
| | R3 | |
| 7 | AB TL1A_HL2_VH | QVQLVQSGAEVKKPGASVKVSCKASGYDFTYYGISWVRQ |
| | | APGQGLEWMGWISPYNGNTHYAQKLQGRVTMTTDTSTRT |
| | | AYMELRSLRSDDTAVYYCARENYYGSGAYRGGMDVWGQ |
| | | GTTVTVSS |
| 8 | AB_TL1A_HL2_VL | EIVLTQSPATLSLSPGERATLSCRASQSVSSYLAWYQQKPG |
| | | QAPRLLIYDASNRATGIPARFSGSGSGTDFTLTISSLEPEDFA |
| | | VYYCQQRSSWPWTFGQGTKVEIK |
| 26 | AB_TL1A_HL2_HC | QVQLVQSGAEVKKPGASVKVSCKASGYDFTYYGISWVRQ |
| | | APGQGLEWMGWISPYNGNTHYAQKLQGRVTMTTDTSTRT |
| | | AYMELRSLRSDDTAVYYCARENYYGSGAYRGGMDVWGQ |
| | | GTTVTVSSASTKGPSVFPLAPSSKSTSGGTAALGCLVKDYF |
| | | PEPVTVSWNSGALTSGVHTFPAVLQSSGLYSLSSVVTVPSS |
| | | SLGTQTYICNVNHKPSNTKVDKKVEPKSCDKTHTCPPCPAP |
| | | EAAGAPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALPAPIEKTISKAKGQPREPQVYTLPP |
| | | SREEMTKNQVSLTCLVKGFYPSDIAVEWESNGQPENNYKT |
| | | TPPVLDSDGSFFLYSKLTVDKSRWQQGNVFSCSVLHEALH |
| | | SHYTQKSLSLSPG |
| 10 | AB_TL1A_HL2_LC | EIVLTQSPATLSLSPGERATLSCRASQSVSSYLAWYQQKPG |
| | | QAPRLLIYDASNRATGIPARFSGSGSGTDFTLTISSLEPEDFA |
| | | VYYCQQRSSWPWTFGQGTKVEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 25 | AB_TL1A_HL9_002_ | YYGIS |
| | HCDR1 | |
| 2 | AB_TL1A_HL9_002 | WISPYNGNTHYAQKLQG |
| | HCDR2 | |
| 27 | AB_TL1A_HL9_002_ | EDYYGSGSYRGGMDV |
| | HCDR3 | |
| 4 | AB_TL1A_HL9_002_ | RASQSVSSYLA |
| | LCDR1 | |
| 5 | AB_TL1A_HL9_002_ | DASNRAT |
| | LCDR2 | |
| 6 | AB_TL1A_HL9_002_ | QQRSSWPWT |
| | LCDR3 | |
| 28 | AB_TL1A_HL9_002_ | QVQLVQSGAEVKKPGASVKVSCKASGYDFTYYGISWVRQ |
| | VH | APGQGLEWMGWISPYNGNTHYAQKLQGRVTMTTDTSTRT |
| | | AYMELRSLRSDDTAVYYCAREDYYGSGSYRGGMDVWGQ |
| | | GTTVTVSS |
| 20 | AB_TL1A_HL9_002_ | EIVLTQSPATLSLSPGERATLSCRASQSVSSYLAWYQQKPG |
| | VL | QAPRLLIYDASTRATGIPARFSGSGSGTDFTLTISSLEPEDFA |
| | | VYYCQQRSNWPWTFGQGTKVEIK |
| 29 | AB_TL1A_HL9_002_ | QVQLVQSGAEVKKPGASVKVSCKASGYDFTYYGISWVRQ |
| | HC | APGQGLEWMGWISPYNGNTHYAQKLQGRVTMTTDTSTRT |
| | | AYMELRSLRSDDTAVYYCAREDYYGSGSYRGGMDVWGQ |
| | | GTTVTVSSASTKGPSVFPLAPSSKSTSGGTAALGCLVKDYF |
| | | PEPVTVSWNSGALTSGVHTFPAVLQSSGLYSLSSVVTVPSS |
| | | SLGTQTYICNVNHKPSNTKVDKKVEPKSCDKTHTCPPCPAP |
| | | EAAGAPSVFLFPPKPKDTLMISRTPEVTCVVVDVSHEDPEV |
| | | KFNWYVDGVEVHNAKTKPREEQYNSTYRVVSVLTVLHRD |
| | | WLNGKEYKCKVSNKALPAPIEKTISKAKGQPREPQVYTLPP |
| | | SREEMTKNQVSLTCLVKGFYPSDIAVEWESNGQPENNYKT |
| | | TPPVLDSDGSFFLYSKLTVDKSRWQQGNVFSCSVLHEALK |
| | | FHYTQKSLSLSPG |
| 21 | AB_TL1A_HL9_002_ | EIVLTQSPATLSLSPGERATLSCRASQSVSSYLAWYQQKPG |
| | LC | QAPRLLIYDASNRATGIPARFSGSGSGTDFTLTISSLEPEDFA |
| | | VYYCQQRSSWPWTFGQGTKVEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |

TABLE 2
Annotated amino acid sequences of anti-IL1β antibodies

| SEQ | Name (of antibody | |
|---|---|---|
| ID NO | and domain or region) | Sequence |
| 43 | AB_IL1b_02_003_HL9 | DHYMS |
| | HCDR1 | |
| 44 | AB_IL1b_02_003_HL9 | YISSSGSTIYYADSVKG |
| | HCDR2 | |
| 45 | AB_IL1b_02_003_HL9 | EYDSSGYYYVGVDF |
| | HCDR3 | |
| 46 | AB_IL1b_02_003_HL9 | RASQSISSYLN |
| | LCDR1 | |
| 47 | AB_IL1b_02_003_HL9 | GASSDQS |
| | LCDR2 | |
| 48 | AB_IL1b_02_003_HL9 | QQGYYFPPT |
| | LCDR3 | |
| 49 | AB_IL1b_02_003_HL | EVQLVESGGGLVQPGGSLRLSCAASGFTFSDHYMSWIRQA |
| | VH | PGKGLEWVSYISSSGSTIYYADSVKGRFTISRDNAKNSLYL |
| | | QMNSLRAEDTAVYYCAREYDSSGYYYVGVDFWGQGTLV |
| | | TVSS |
| 50 | AB_IL1b_02_003_HL9 | DIQMTQSPSSLSASVGDRVTITCRASQSISSYLNWYQQKPG |
| | VL | KAPKLLIYGASSDQSGVPSRFSGSGSGTDFTLTISSLQPEDF |
| | | ATYYCQQGYYFPPTFGGGTKVEIK |
| 51 | AB_IL1b_02_003_HL9 | EVQLVESGGGLVQPGGSLRLSCAASGFTFSDHYMSWIRQA |
| | HC | PGKGLEWVSYISSSGSTIYYADSVKGRFTISRDNAKNSLYL |
| | | QMNSLRAEDTAVYYCAREYDSSGYYYVGVDFWGQGTLV |
| | | TVSSASTKGPSVFPLAPCSRSTSESTAALGCLVKDYFPEPVT |
| | | VSWNSGALTSGVHTFPAVLQSSGLYSLSSVVTVPSSSLGTK |
| | | TYTCNVDHKPSNTKVDKRVESKYGPPCPPCPAPEAAGGPS |
| | | VFLFPPKPKDTLMISRTPEVTCVVVDVSQEDPEVQFNWYV |
| | | DGVEVHNAKTKPREEQFNSTYRVVSVLTVLHRDWLNGKE |
| | | YKCKVSNKGLPSSIEKTISKAKGQPREPQVYTLPPSQEEMT |
| | | KNQVSLTCLVKGFYPSDIAVEWESNGQPENNYKTTPPVLD |
| | | SDGSFFLYSRLTVDKSRWQEGNVFSCSVLHEALKFHYTQK |
| | | SLSLSLG |
| 52 | AB_IL1b_02_003_HL9 | DIQMTQSPSSLSASVGDRVTITCRASQSISSYLNWYQQKPG |
| | LC | KAPKLLIYGASSDQSGVPSRFSGSGSGTDFTLTISSLQPEDF |
| | | ATYYCQQGYYFPPTFGGGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC |

TABLE 3
Annotated amino acid sequences of anti-IL13 antibodies

| SEQ | Name (of antibody | |
|---|---|---|
| ID NO | and domain or region) | Sequence |
| 53 | AB_IL13_EE_02_001_ | AYSVN |
| | HL9 HCDR1 | |
| 54 | AB_IL13_EE_02_001_ | MIWGDGKIVYNSALKS |
| | HL9 HCDR2 | |
| 55 | AB_IL13_EE_02_001_ | DGYYPYFMDN |
| | HL9 HCDR3 | |
| 56 | AB_IL13_EE_02_001_ | RASKSVDSYGNSFMH |
| | HL9 LCDR1 | |
| 57 | AB_IL13_EE_02_001_ | LASNLES |
| | HL9 LCDR2 | |
| 58 | AB_IL13_EE_02_001_ | QQNNEDPRT |
| | HL9 LCDR3 | |
| 59 | AB_IL13_EE_02_001_ | EVQLQESGPGLVKPSETLSLTCTVSGFSLNAYSVNWIRQPP |
| | HL9 VH | GKGLEWLGMIWGDGKIVYNSALKSRLTISKDSSKNQVSLK |
| | | LSSVTAADTAVYYCAGDGYYPYFMDNWGQGTTVTVSS |
| 60 | AB_IL13_EE_02_001_ | DIQLTQSPSSLSASVGDRVTITCRASKSVDSYGNSFMHWYQ |
| | HL9 VL | QKPGKAPKLLIYLASNLESGVPSRFSGSGSGTDFTLTISSLQP |
| | | EDFATYYCQQNNEDPRTFGGGTKVEIK |
| 61 | AB_IL13_EE_02_001_ | EVQLQESGPGLVKPSETLSLTCTVSGFSLNAYSVNWIRQPP |
| | HL9 HC | GKGLEWLGMIWGDGKIVYNSALKSRLTISKDSSKNQVSLK |
| | | LSSVTAADTAVYYCAGDGYYPYFMDNWGQGTTVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICNV |
| | | NHKPSNTKVDKKVEPKSCDKTHTCPPCPAPEAAGGPSVFLF |
| | | PPKPKDTLMISRTPEVTCVVVDVSHEDPEVKFNWYVDGVE |
| | | VHNAKTKPREEQYNSTYRVVSVLTVLHRDWLNGKEYKCK |
| | | VSNKALPAPIEKTISKAKGQPREPQVYTLPPSRDELTKNQVS |
| | | LTCLVKGFYPSDIAVEWESNGQPENNYKTTPPVLDSDGSFF |
| | | LYSKLTVDKSRWQQGNVFSCSVLHEALKFHYTQKSLSLSP |
| | | G |
| 62 | AB_IL13_EE_02_001_ | DIQLTQSPSSLSASVGDRVTITCRASKSVDSYGNSFMHWYQ |
| | HL9 LC | QKPGKAPKLLIYLASNLESGVPSRFSGSGSGTDFTLTISSLQP |
| | | EDFATYYCQQNNEDPRTFGGGTKVEIKRTVAAPSVFIFPPS |
| | | DEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 53 | AB_IL13_EE_02_004_ | AYSVN |
| | HL9 HCDR1 | |
| 54 | AB_IL13_EE_02_004_ | MIWGDGKIVYNSALKS |
| | HL9 HCDR2 | |
| 63 | AB_IL13_EE_02_004_ | DGYYPYFMDN |
| | HL9 HCDR3 | |
| 56 | AB_IL13_EE_02_004_ | RASKSVDSYGNSFMH |
| | HL9 LCDR1 | |
| 57 | AB_IL13_EE_02_004_ | LASNLES |
| | HL9 LCDR2 | |
| 58 | AB_IL13_EE_02_004_ | QQNNEDPRT |
| | HL9 LCDR3 | |
| 64 | AB_IL13_EE_02_004_ | EVQLQESGPGLVKPSETLSLTCTVSGFSLNAYSVNWIRQPP |
| | HL9 VH | GKGLEWLGMIWGDGKIVYNSALKSRLTISKDSSKNQVSLK |
| | | LSSVTAADTAVYYCAGDGYYPYAMDGWGQGTTVTVSS |
| 60 | AB_IL13_EE_02_004_ | DIQLTQSPSSLSASVGDRVTITCRASKSVDSYGNSFMHWYQ |
| | HL9 VL | QKPGKAPKLLIYLASNLESGVPSRFSGSGSGTDFTLTISSLQP |
| | | EDFATYYCQQNNEDPRTFGGGTKVEIK |
| 65 | AB_IL13_EE_02_004_ | EVQLQESGPGLVKPSETLSLTCTVSGFSLNAYSVNWIRQPP |
| | HL9 HC | GKGLEWLGMIWGDGKIVYNSALKSRLTISKDSSKNQVSLK |
| | | LSSVTAADTAVYYCAGDGYYPYAMDGWGQGTTVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICNV |
| | | NHKPSNTKVDKKVEPKSCDKTHTCPPCPAPEAAGGPSVFLF |
| | | PPKPKDTLMISRTPEVTCVVVDVSHEDPEVKFNWYVDGVE |
| | | VHNAKTKPREEQYNSTYRVVSVLTVLHRDWLNGKEYKCK |
| | | VSNKALPAPIEKTISKAKGQPREPQVYTLPPSRDELTKNQVS |
| | | LTCLVKGFYPSDIAVEWESNGQPENNYKTTPPVLDSDGSFF |
| | | LYSKLTVDKSRWQQGNVFSCSVLHEALKFHYTQKSLSLSP |
| | | G |
| 62 | AB_IL13_EE_02_004_ | DIQLTQSPSSLSASVGDRVTITCRASKSVDSYGNSFMHWYQ |
| | HL9 LC | QKPGKAPKLLIYLASNLESGVPSRFSGSGSGTDFTLTISSLQP |
| | | EDFATYYCQQNNEDPRTFGGGTKVEIKRTVAAPSVFIFPPS |
| | | DEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |

TABLE 4
Annotated amino acid sequences of anti-IL15 antibodies

| SEQ | Name (of antibody | |
|---|---|---|
| ID NO | and domain or region) | Sequence |
| 66 | AB_IL15_AI_02_077_ | NYAMS |
| | HL9 HCDR1 | |
| 67 | AB_IL15_AI_02_077_ | TISRGGDYHYYPDSVKG |
| | HL9 HCDR2 | |
| 68 | AB_IL15_AI_02_077_ | RVSMITGGWAHDY |
| | HL9 HCDR3 | |
| 69 | AB_IL15_AI_02_077_ | RSSQSIVDITGNTYLE |
| | HL9 LCDR1 | |
| 70 | AB_IL15_AI_02_077_ | KVFNRFS |
| | HL9 LCDR2 | |
| 71 | AB_IL15_AI_02_077_ | FQDSFVPYT |
| | HL9 LCDR3 | |
| 72 | AB_IL15_AI_02_077_ | EVQLVESGGGLVQPGGSLRLSCAASEFTFSNYAMSWVRQA |
| | HL VH | PGKGLEWVATISRGGDYHYYPDSVKGRFTISRDNSKNTLY |
| | | LQMNSLRAEDTAVYYCARRVSMITGGWAHDYWGQGTLV |
| | | TVSS |
| 73 | AB_IL15_AI_02_077_ | DVVMTQSPLSLPVTLGQPASISCRSSQSIVDITGNTYLEWYQ |
| | HL9 VL | QRPGQSPRLLIYKVFNRFSGVPDRFSGSGSGTDFTLKISRVE |
| | | AEDVGVYYCFQDSFVPYTFGQGTKLEIK |
| 74 | AB_IL15_AI_02_077_ | EVQLVESGGGLVQPGGSLRLSCAASEFTFSNYAMSWVRQA |
| | HL9 HC | PGKGLEWVATISRGGDYHYYPDSVKGRFTISRDNSKNTLY |
| | | LQMNSLRAEDTAVYYCARRVSMITGGWAHDYWGQGTLV |
| | | TVSSASTKGPSVFPLAPSSKSTSGGTAALGCLVKDYFPEPVT |
| | | VSWNSGALTSGVHTFPAVLQSSGLYSLSSVVTVPSSSLGTQ |
| | | TYICNVNHKPSNTKVDKRVEPKSCDKTHTCPPCPAPEAAG |
| | | GPSVFLFPPKPKDTLMISRTPEVTCVVVDVSHEDPEVKFNW |
| | | YVDGVEVHNAKTKPREEQYNSTYRVVSVLTVLHRDWLNG |
| | | KEYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVLHEALKFHYT |
| | | QKSLSLSPGK |
| 75 | AB_IL15_AI_02_077_ | DVVMTQSPLSLPVTLGQPASISCRSSQSIVDITGNTYLEWYQ |
| | HL9 LC | QRPGQSPRLLIYKVFNRFSGVPDRFSGSGSGTDFTLKISRVE |
| | | AEDVGVYYCFQDSFVPYTFGQGTKLEIKRTVAAPSVFIFPPS |
| | | DEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |

TABLE 5
Annotated amino acid sequences of bispecific anti-IL13/anti-TSLP antibodies

| SEQ | Name (of antibody | |
|---|---|---|
| ID NO | and domain or region) | Sequence |
| 76 | AB_IL13_TSLP_BsAb_ | NYLIE |
| | ISTF_003_HL9 TSLP | |
| | HCDR1 | |
| 77 | AB_IL13_TSLP_BsAb_ | VIDPGVGDTNYNENFKG |
| | ISTF_003_HL9 TSLP | |
| | HCDR2 | |
| 78 | AB_IL13_TSLP_BsAb_ | EDNPGTAFDY |
| | ISTF_003_HL9 TSLP | |
| | HCDR3 | |
| 79 | AB_IL13_TSLP_BsAb_ | KASQSVSSDVT |
| | ISTF_003_HL9 TSLP | |
| | LCDR1 | |
| 80 | AB_IL13_TSLP_BsAb_ | YVSEHYT |
| | ISTF_003_HL9 TSLP | |
| | LCDR2 | |
| 81 | AB_IL13_TSLP_BsAb_ | QQHHRFPLT |
| | ISTF_003_HL9 TSLP | |
| | LCDR3 | |
| 53 | AB_IL13_TSLP_BsAb_ | AYSVN |
| | ISTF_003_IL13 | |
| | HCDR1 | |
| 54 | AB_IL13_TSLP_BsAb_ | MIWGDGKIVYNSALKS |
| | ISTF_003_IL13 | |
| | HCDR2 | |
| 82 | AB_IL13_TSLP_BsAb_ | DGYYPYAMRN |
| | ISTF_003_IL13 | |
| | HCDR3 | |
| 56 | AB_IL13_TSLP_BsAb_ | RASKSVDSYGNSFMH |
| | ISTF_003_IL13 | |
| | LCDR1 | |
| 57 | AB_IL13_TSLP_BsAb_ | LASNLES |
| | ISTF_003_IL13 | |
| | LCDR2 | |
| 58 | AB_IL13_TSLP_BsAb_ | QQNNEDPRT |
| | ISTF_003_IL13 | |
| | LCDR3 | |
| 84 | AB_IL13_TSLP_BsAb_ | EVQLVQSGAEVKKPGSSVKVSCKASGYTFSNYLIEWVRQA |
| | ISTF_003_TSLP VH | PGQGLEWIGVIDPGVGDTNYNENFKGRATLTADKSTSTAYI |
| | | ELSSLRSEDTAVYYCAREDNPGTAFDYWGQGTTVTVSS |
| 85 | AB_IL13_TSLP_BsAb_ | SIVMTQTPLSLSVTPGQPASISCKASQSVSSDVTWYLQKPG |
| | ISTF_003_TSLP VL | QSPQLLIYYVSEHYTGVPDRFSGSGYGTDFTLKISRVEAED |
| | | VGVYYCQQHHRFPLTFGQGTKLEIK |
| 86 | AB_IL13_TSLP_BsAb_ | EVQLQESGPGLVKPSETLSLTCTVSGFSLNAYSVNWIRQPP |
| | ISTF_003_IL13 VH | GKCLEWLGMIWGDGKIVYNSALKSRLTISKDSSKNQVSLK |
| | | LSSVTAADTAVYYCAGDGYYPYAMRNWGQGTTVTVSS |
| 87 | AB_IL13_TSLP_BsAb_ | DIQLTQSPSSLSASVGDRVTITCRASKSVDSYGNSFMHWYQ |
| | ISTF_003_IL13 VL | QKPGKAPKLLIYLASNLESGVPSRFSGSGSGTDFTLTISSLQP |
| | | EDFATYYCQQNNEDPRTFGCGTKVEIK |
| 88 | AB_IL13_TSLP_BsAb_ | EVQLVQSGAEVKKPGSSVKVSCKASGYTFSNYLIEWVRQA |
| | ISTF_003_HC | PGQGLEWIGVIDPGVGDTNYNENFKGRATLTADKSTSTAYI |
| | | ELSSLRSEDTAVYYCAREDNPGTAFDYWGQGTTVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICNV |
| | | NHKPSNTKVDKRVEPKSCDKTHTCPPCPAPEAAGGPSVFLF |
| | | PPKPKDTLMISRTPEVTCVVVDVSHEDPEVKFNWYVDGVE |
| | | VHNAKTKPREEQYNSTYRVVSVLTVLHRDWLNGKEYKCK |
| | | VSNKALPAPIEKTISKAKGQPREPQVYTLPPCREEMTKNQV |
| | | SLWCLVKGFYPSDIAVEWESNGQPENNYKTTPPVLDSDGS |
| | | FFLYSKLTVDKSRWQQGNVFSCSVLHEALKFHYTQKSLSL |
| | | SPGK |
| 89 | AB_IL13_TSLP_BsAb_ | SIVMTQTPLSLSVTPGQPASISCKASQSVSSDVTWYLQKPG |
| | ISTF_003_LC | QSPQLLIYYVSEHYTGVPDRFSGSGYGTDFTLKISRVEAED |
| | | VGVYYCQQHHRFPLTFGQGTKLEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| 90 | AB_IL13_TSLP_BsAb_ | DIQLTQSPSSLSASVGDRVTITCRASKSVDSYGNSFMHWYQ |
| | ISTF_003_ScFv-Fc | QKPGKAPKLLIYLASNLESGVPSRFSGSGSGTDFTLTISSLQP |
| | | EDFATYYCQQNNEDPRTFGCGTKVEIKGGGGSGGGGSGGG |
| | | GSGGGGSEVQLQESGPGLVKPSETLSLTCTVSGFSLNAYSV |
| | | NWIRQPPGKCLEWLGMIWGDGKIVYNSALKSRLTISKDSS |
| | | KNQVSLKLSSVTAADTAVYYCAGDGYYPYAMRNWGQGT |
| | | TVTVSSEPKSSDKTHTCPPCPAPEAAGGPSVFLFPPKPKDTL |
| | | MISRTPEVTCVVVDVSHEDPEVKFNWYVDGVEVHNAKTK |
| | | PREEQYNSTYRVVSVLTVLHRDWLNGKEYKCKVSNKALP |
| | | APIEKTISKAKGQPREPQVCTLPPSREEMTKNQVSLSCAVK |
| | | GFYPSDIAVEWESNGQPENNYKTTPPVLDSDGSFFLVSKLT |
| | | VDKSRWQQGNVFSCSVLHEALKFHYTQKSLSLSPGK |

TABLE 6
Annotated amino acid sequences of Comparator Antibodies

| SEQ ID | | |
|---|---|---|
| NO | Domain or Region | Sequence |
| 25 | RVT-3101 Heavy | YYGIS |
| | chain CDR1 | |
| 30 | RVT-3101 Heavy | WISTYNGNTHYARMLQG |
| | chain CDR2 | |
| 3 | RVT-3101 Heavy | ENYYGSGAYRGGMDV |
| | chain CDR3 | |
| 4 | RVT-3101 Light chain | RASQSVSSYLA |
| | CDR1 | |
| 5 | RVT-3101 Light chain | DASNRAT |
| | CDR2 | |
| 12 | RVT-3101 Light chain | QQRSNWPWT |
| | CDR3 | |
| 31 | RVT-3101 VH | QVQLVQSGAEVKKPGASVKVSCKASGYDFTYYGISWV |
| | | RQAPGQGLEWMGWISTYNGNTHYARMLQGRVTMTTD |
| | | TSTRTAYMELRSLRSDDTAVYYCARENYYGSGAYRGG |
| | | MDVWGQGTTVTVSS |
| 14 | RVT-3101 VL | EIVLTQSPATLSLSPGERATLSCRASQSVSSYLAWYQQK |
| | | PGQAPRLLIYDASNRATGIPARFSGSGSGTDFTLTISSLEP |
| | | EDFAVYYCQQRSNWPWTFGQGTKVEIK |
| 32 | RVT-3101 HC | QVQLVQSGAEVKKPGASVKVSCKASGYDFTYYGISWV |
| | | RQAPGQGLEWMGWISTYNGNTHYARMLQGRVTMTTD |
| | | TSTRTAYMELRSLRSDDTAVYYCARENYYGSGAYRGG |
| | | MDVWGQGTTVTVSSASTKGPSVFPLAPSSKSTSGGTAA |
| | | LGCLVKDYFPEPVTVSWNSGALTSGVHTFPAVLQSSGL |
| | | YSLSSVVTVPSSSLGTQTYICNVNHKPSNTKVDKKVEPK |
| | | SCDKTHTCPPCPAPEAAGAPSVFLFPPKPKDTLMISRTPE |
| | | VTCVVVDVSHEDPEVKFNWYVDGVEVHNAKTKPREEQ |
| | | YNSTYRVVSVLTVLHQDWLNGKEYKCKVSNKALPAPIE |
| | | KTISKAKGQPREPQVYTLPPSREEMTKNQVSLTCLVKGF |
| | | YPSDIAVEWESNGQPENNYKTTPPVLDSDGSFFLYSKLT |
| | | VDKSRWQQGNVFSCSVMHEALHNHYTQKSLSLSPG |
| 16 | RVT-3101 LC | EIVLTQSPATLSLSPGERATLSCRASQSVSSYLAWYQQK |
| | | PGQAPRLLIYDASNRATGIPARFSGSGSGTDFTLTISSLEP |
| | | EDFAVYYCQQRSNWPWTFGQGTKVEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 43 | AVTX-009 Heavy | DHYMS |
| | chain CDR1 | |
| 44 | AVTX-009 Heavy | YISSSGSTIYYADSVKG |
| | chain CDR2 | |
| 111 | AVTX-009 Heavy | EADSSGYYYVGVDV |
| | chain CDR3 | |
| 46 | AVTX-009 Light | RASQSISSYLN |
| | chain CDR1 | |
| 47 | AVTX-009 Light | GASSDQS |
| | chain CDR2 | |
| 48 | AVTX-009 Light | QQGYYFPPT |
| | chain CDR3 | |
| 53 | APG-777 Heavy chain | AYSVN |
| | CDR1 | |
| 54 | APG-777 Heavy chain | MIWGDGKIVYNSALKS |
| | CDR2 | |
| 112 | APG-777 Heavy chain | DGYYPYAMDN |
| | CDR3 | |
| 56 | APG-777 Light chain | RASKSVDSYGNSFMH |
| | CDR1 | |
| 57 | APG-777 Light chain | LASNLES |
| | CDR2 | |
| 58 | APG-777 Light chain | QQNNEDPRT |
| | CDR3 | |
| 76 | AIO-001 Heavy chain | NYLIE |
| | CDR1 | |
| 77 | AIO-001 Heavy chain | VIDPGVGDTNYNENFKG |
| | CDR2 | |
| 113 | AIO-001 Heavy chain | EDNTGTAFDY |
| | CDR3 | |
| 79 | AIO-001 Light chain | KASQSVSSDVT |
| | CDR1 | |
| 80 | AIO-001 Light chain | YVSEHYT |
| | CDR2 | |
| 81 | AIO-001 Light chain | QQHHRFPLT |
| | CDR3 | |
| 66 | CALY-002 Heavy | NYAMS |
| | chain CDR1 | |
| 114 | CALY-002 Heavy | TISRGGDYTYYPDSVKG |
| | chain CDR2 | |
| 115 | CALY-002 Heavy | RVSMITGGWAMDY |
| | chain CDR3 | |
| 69 | CALY-002 Light | RSSQSIVDITGNTYLE |
| | chain CDR1 | |
| 70 | CALY-002 Light | KVFNRFS |
| | chain CDR2 | |
| 71 | CALY-002 Light | FQDSFVPYT |
| | chain CDR3 | |

TABLE 7
Exemplary antibody constant region amino acid sequences

| SEQ ID | | |
|---|---|---|
| NO | Domain or Region | Sequence |
| 33 | Heavy chain constant | ASTKGPSVFPLAPSSKSTSGGTAALGCLVKDYFPEPVTV |
| | region | SWNSGALTSGVHTFPAVLQSSGLYSLSSVVTVPSSSLGT |
| | | QTYICNVNHKPSNTKVDKKVEPKSCDKTHTCPPCPAPEA |
| | | AGAPSVFLFPPKPKDTLMISRTPEVTCVVVDVSHEDPEV |
| | | KFNWYVDGVEVHNAKTKPREEQYNSTYRVVSVLTVLH |
| | | QDWLNGKEYKCKVSNKALPAPIEKTISKAKGQPREPQV |
| | | YTLPPSREEMTKNQVSLTCLVKGFYPSDIAVEWESNGQP |
| | | ENNYKTTPPVLDSDGSFFLYSKLTVDKSRWQQGNVFSC |
| | | SVMHEALHNHYTQKSLSLSPG |
| 34 | Heavy chain constant | ASTKGPSVFPLAPSSKSTSGGTAALGCLVKDYFPEPVTV |
| | region | SWNSGALTSGVHTFPAVLQSSGLYSLSSVVTVPSSSLGT |
| | | QTYICNVNHKPSNTKVDKKVEPKSCDKTHTCPPCPAPEA |
| | | AGAPSVFLFPPKPKDTLMISRTPEVTCVVVDVSHEDPEV |
| | | KFNWYVDGVEVHNAKTKPREEQYNSTYRVVSVLTVLH |
| | | QDWLNGKEYKCKVSNKALPAPIEKTISKAKGQPREPQV |
| | | YTLPPSREEMTKNQVSLTCLVKGFYPSDIAVEWESNGQP |
| | | ENNYKTTPPVLDSDGSFFLYSKLTVDKSRWQQGNVFSC |
| | | SVLHEALHSHYTQKSLSLSPG |
| 35 | Heavy chain constant | ASTKGPSVFPLAPSSKSTSGGTAALGCLVKDYFPEPVTV |
| | region | SWNSGALTSGVHTFPAVLQSSGLYSLSSVVTVPSSSLGT |
| | | QTYICNVNHKPSNTKVDKKVEPKSCDKTHTCPPCPAPEA |
| | | AGAPSVFLFPPKPKDTLMISRTPEVTCVVVDVSHEDPEV |
| | | KFNWYVDGVEVHNAKTKPREEQYNSTYRVVSVLTVLH |
| | | RDWLNGKEYKCKVSNKALPAPIEKTISKAKGQPREPQV |
| | | YTLPPSREEMTKNQVSLTCLVKGFYPSDIAVEWESNGQP |
| | | ENNYKTTPPVLDSDGSFFLYSKLTVDKSRWQQGNVFSC |
| | | SVLHEALKFHYTQKSLSLSPG |
| 95 | Heavy chain constant | ASTKGPSVFPLAPSSKSTSGGTAALGCLVKDYFPEPVTV |
| | region Q311 | SWNSGALTSGVHTFPAVLQSSGLYSLSSVVTVPSSSLGT |
| | | QTYICNVNHKPSNTKVDKKVEPKSCDKTHTCPPCPAPEA |
| | | AGAPSVFLFPPKPKDTLMISRTPEVTCVVVDVSHEDPEV |
| | | KFNWYVDGVEVHNAKTKPREEQYNSTYRVVSVLTVLH |
| | | QDWLNGKEYKCKVSNKALPAPIEKTISKAKGQPREPQV |
| | | YTLPPSREEMTKNQVSLTCLVKGFYPSDIAVEWESNGQP |
| | | ENNYKTTPPVLDSDGSFFLYSKLTVDKSRWQQGNVFSC |
| | | SVLHEALKFHYTQKSLSLSPG |
| 96 | Heavy chain constant | ASTKGPSVFPLAPSSKSTSGGTAALGCLVKDYFPEPVTV |
| | region M428 | SWNSGALTSGVHTFPAVLQSSGLYSLSSVVTVPSSSLGT |
| | | QTYICNVNHKPSNTKVDKKVEPKSCDKTHTCPPCPAPEA |
| | | AGAPSVFLFPPKPKDTLMISRTPEVTCVVVDVSHEDPEV |
| | | KFNWYVDGVEVHNAKTKPREEQYNSTYRVVSVLTVLH |
| | | RDWLNGKEYKCKVSNKALPAPIEKTISKAKGQPREPQV |
| | | YTLPPSREEMTKNQVSLTCLVKGFYPSDIAVEWESNGQP |
| | | ENNYKTTPPVLDSDGSFFLYSKLTVDKSRWQQGNVFSC |
| | | SVMHEALKFHYTQKSLSLSPG |
| 97 | Heavy chain constant | ASTKGPSVFPLAPSSKSTSGGTAALGCLVKDYFPEPVTV |
| | region H433 | SWNSGALTSGVHTFPAVLQSSGLYSLSSVVTVPSSSLGT |
| | | QTYICNVNHKPSNTKVDKKVEPKSCDKTHTCPPCPAPEA |
| | | AGAPSVFLFPPKPKDTLMISRTPEVTCVVVDVSHEDPEV |
| | | KFNWYVDGVEVHNAKTKPREEQYNSTYRVVSVLTVLH |
| | | RDWLNGKEYKCKVSNKALPAPIEKTISKAKGQPREPQV |
| | | YTLPPSREEMTKNQVSLTCLVKGFYPSDIAVEWESNGQP |
| | | ENNYKTTPPVLDSDGSFFLYSKLTVDKSRWQQGNVFSC |
| | | SVLHEALHFHYTQKSLSLSPG |
| 98 | Heavy chain constant | ASTKGPSVFPLAPSSKSTSGGTAALGCLVKDYFPEPVTV |
| | region N434 | SWNSGALTSGVHTFPAVLQSSGLYSLSSVVTVPSSSLGT |
| | | QTYICNVNHKPSNTKVDKKVEPKSCDKTHTCPPCPAPEA |
| | | AGAPSVFLFPPKPKDTLMISRTPEVTCVVVDVSHEDPEV |
| | | KFNWYVDGVEVHNAKTKPREEQYNSTYRVVSVLTVLH |
| | | RDWLNGKEYKCKVSNKALPAPIEKTISKAKGQPREPQV |
| | | YTLPPSREEMTKNQVSLTCLVKGFYPSDIAVEWESNGQP |
| | | ENNYKTTPPVLDSDGSFFLYSKLTVDKSRWQQGNVFSC |
| | | SVLHEALKNHYTQKSLSLSPG |
| 128 | AB_IL1b_02_003_HL9 | ASTKGPSVFPLAPCSRSTSESTAALGCLVKDYFPEPVTVS |
| | Heavy Chain | WNSGALTSGVHTFPAVLQSSGLYSLSSVVTVPSSSLGTK |
| | Constant | TYTCNVDHKPSNTKVDKRVESKYGPPCPPCPAPEAAGG |
| | | PSVFLFPPKPKDTLMISRTPEVTCVVVDVSQEDPEVQFN |
| | | WYVDGVEVHNAKTKPREEQFNSTYRVVSVLTVLHRDW |
| | | LNGKEYKCKVSNKGLPSSIEKTISKAKGQPREPQVYTLP |
| | | PSQEEMTKNQVSLTCLVKGFYPSDIAVEWESNGQPENN |
| | | YKTTPPVLDSDGSFFLYSRLTVDKSRWQEGNVFSCSVLH |
| | | EALKFHYTQKSLSLSLG |
| 129 | AB_IL13_EE_02_001_ | ASTKGPSVFPLAPSSKSTSGGTAALGCLVKDYFPEPVTV |
| | HL9 Heavy Chain | SWNSGALTSGVHTFPAVLQSSGLYSLSSVVTVPSSSLGT |
| | Constant | QTYICNVNHKPSNTKVDKKVEPKSCDKTHTCPPCPAPEA |
| | | AGGPSVFLFPPKPKDTLMISRTPEVTCVVVDVSHEDPEV |
| | | KFNWYVDGVEVHNAKTKPREEQYNSTYRVVSVLTVLH |
| | | RDWLNGKEYKCKVSNKALPAPIEKTISKAKGQPREPQV |
| | | YTLPPSRDELTKNQVSLTCLVKGFYPSDIAVEWESNGQP |
| | | ENNYKTTPPVLDSDGSFFLYSKLTVDKSRWQQGNVFSC |
| | | SVLHEALKFHYTQKSLSLSPG |
| 130 | AB_IL13_EE_02_004_ | ASTKGPSVFPLAPSSKSTSGGTAALGCLVKDYFPEPVTV |
| | HL9 Heavy Chain | SWNSGALTSGVHTFPAVLQSSGLYSLSSVVTVPSSSLGT |
| | Constant | QTYICNVNHKPSNTKVDKKVEPKSCDKTHTCPPCPAPEA |
| | | AGGPSVFLFPPKPKDTLMISRTPEVTCVVVDVSHEDPEV |
| | | KFNWYVDGVEVHNAKTKPREEQYNSTYRVVSVLTVLH |
| | | RDWLNGKEYKCKVSNKALPAPIEKTISKAKGQPREPQV |
| | | YTLPPSRDELTKNQVSLTCLVKGFYPSDIAVEWESNGQP |
| | | ENNYKTTPPVLDSDGSFFLYSKLTVDKSRWQQGNVFSC |
| | | SVLHEALKFHYTQKSLSLSPG |
| 131 | AB_IL13_TSLP_BsAb_ | ASTKGPSVFPLAPSSKSTSGGTAALGCLVKDYFPEPVTV |
| | ISTF_003_HL9 | SWNSGALTSGVHTFPAVLQSSGLYSLSSVVTVPSSSLGT |
| | Heavy Chain Constant | QTYICNVNHKPSNTKVDKRVEPKSCDKTHTCPPCPAPEA |
| | | AGGPSVFLFPPKPKDTLMISRTPEVTCVVVDVSHEDPEV |
| | | KFNWYVDGVEVHNAKTKPREEQYNSTYRVVSVLTVLH |
| | | RDWLNGKEYKCKVSNKALPAPIEKTISKAKGQPREPQV |
| | | YTLPPCREEMTKNQVSLWCLVKGFYPSDIAVEWESNGQ |
| | | PENNYKTTPPVLDSDGSFFLYSKLTVDKSRWQQGNVFS |
| | | CSVLHEALKFHYTQKSLSLSPGK |
| 132 | AB_IL15_AI_02_077_ | ASTKGPSVFPLAPSSKSTSGGTAALGCLVKDYFPEPVTV |
| | HL9 Heavy Chain | SWNSGALTSGVHTFPAVLQSSGLYSLSSVVTVPSSSLGT |
| | Constant | QTYICNVNHKPSNTKVDKRVEPKSCDKTHTCPPCPAPEA |
| | | AGGPSVFLFPPKPKDTLMISRTPEVTCVVVDVSHEDPEV |
| | | KFNWYVDGVEVHNAKTKPREEQYNSTYRVVSVLTVLH |
| | | RDWLNGKEYKCKVSNKALPAPIEKTISKAKGQPREPQV |
| | | YTLPPSREEMTKNQVSLTCLVKGFYPSDIAVEWESNGQP |
| | | ENNYKTTPPVLDSDGSFFLYSKLTVDKSRWQQGNVFSC |
| | | SVLHEALKFHYTQKSLSLSPGK |
| 36 | Light chain constant | RTVAAPSVFIFPPSDEQLKSGTASVVCLLNNFYPREAKV |
| | region | QWKVDNALQSGNSQESVTEQDSKDSTYSLSSTLTLSKA |
| | | DYEKHKVYACEVTHQGLSSPVTKSFNRGEC |

TABLE 8
Exemplary Target Antigen sequences

| SEQ ID | | |
|---|---|---|
| NO | Domain or Region | Sequence |
| 37 | Human TL1A | MAEDLGLSFGETASVEMLPEHGSCRPKARSSSARWALTC |
| | | CLVLLPFLAGLTTYLLVSQLRAQGEACVQFQALKGQEFA |
| | | PSHQQVYAPLRADGDKPRAHLTVVRQTPTQHFKNQFPAL |
| | | HWEHELGLAFTKNRMNYTNKFLLIPESGDYFIYSQVTFR |
| | | GMTSECSEIRQAGRPNKPDSITVVITKVTDSYPEPTQLLM |
| | | GTKSVCEVGSNWFQPIYLGAMFSLQEGDKLMVNVSDISL |
| | | VDYTKEDKTFFGAFLL |
| 38 | Rhesus monkey | MAEDLGLSFGETASVEMLPEHGSCRPKARSSSACWALTC |
| | TL1A | CLVLLPFLAGLTTYLLVSQLRAQGEACVQLQ |
| | | DLKGQEFAPSHQQVYAPLRADGDKPRAHLTVVRQTPTQ |
| | | HLKNQFPALHWEHELGLAFTKNRMNYTNKFLL |
| | | IPESGDYFVYSQVTFRGMTSECSEIRQAGRPNKPDSITVVI |
| | | TKVTDSYPEPTQLLMGTKSVCEVGSNWFQ |
| | | PIYLGAMFSLQEGDKLMVNVSDISLVDYTKEDKTFFGAF |
| | | LL |
| 39 | Mouse TL1A | MAEELGLGFGEGVPVEVLPEGCRHRPEARAGLAARSKA |
| | | CLALTCCLLSFPILAGLSTLLMAGQLRVPGKDCMLRAITE |
| | | ERSEPSPQQVYSPPRGKPRAHLTIKKQTPAPHLKNQLSAL |
| | | HWEHDLGMAFTKNGMKYINKSLVIPESGDYFIYSQITFRG |
| | | TTSVCGDISRGRRPNKPDSITMVITKVADSYPEPARLLTGS |
| | | KSVCEISNNWFQSLYLGATFSLEEGDRLMVNVSDISLVD |
| | | YTKEDKTFFGAFLL |
| 116 | Human IL1β | MAEVPELASEMMAYYSGNEDDLFFEADGPKQMKCSFQD |
| | | LDLCPLDGGIQLRISDHHYSKGFRQAASVVVAMDKLRK |
| | | MLVPCPQTFQENDLSTFFPFIFEEEPIFFDTWDNEAYVHD |
| | | APVRSLNCTLRDSQQKSLVMSGPYELKALHLQGQDMEQ |
| | | QVVFSMSFVQGEESNDKIPVALGLKEKNLYLSCVLKDDK |
| | | PTLQLESVDPKNYPKKKMEKRFVFNKIEINNKLEFESAQF |
| | | PNWYISTSQAENMPVFLGGTKGGQDITDFTMQFVSS |
| 117 | Rhesus monkey IL1β | MAEVPELASEMMAYYSGNEDDLFFDVDGPKQMKCSFQ |
| | | DLDLCPLGGGIQLQISHEHYNEGFRQAVSVVVAMEKLRK |
| | | MLVPCPQIFQDNDLSTLIPFIFEEEPVFLDTRNNDACVHDA |
| | | PVRSLHCTLRDAQLKSLVMSGPYELKALHLQGQDLEQQ |
| | | VVFSMSFVQGEESNDKIPVALGLKAKNLYLSCVLKDDKP |
| | | TLQLESVDPKNYPKKKMEKRFVFNKIEINNKLEFESAQFP |
| | | NWYISTSQAENMPVFLGGTRGGQDITDFTMQFVSS |
| 118 | Mouse IL1β | MATVPELNCEMPPFDSDENDLFFEVDGPQKMKGCFQTFD |
| | | LGCPDESIQLQISQQHINKSFRQAVSLIVAVEKLWQLPVSF |
| | | PWTFQDEDMSTFFSFIFEEEPILCDSWDDDDNLLVCDVPI |
| | | RQLHYRLRDEQQKSLVLSDPYELKALHLNGQNINQQVIF |
| | | SMSFVQGEPSNDKIPVALGLKGKNLYLSCVMKDGTPTLQ |
| | | LESVDPKQYPKKKMEKRFVFNKIEVKSKVEFESAEFPNW |
| | | YISTSQAEHKPVFLGNNSGQDIIDFTMESVSS |
| 119 | Human IL13 | MHPLLNPLLLALGLMALLLTTVIALTCLGGFASPGPVPPS |
| | | TALRELIEELVNITQNQKAPLCNGSMVWSINLTAGMYCA |
| | | ALESLINVSGCSAIEKTQRMLSGFCPHKVSAGQFSSLHVR |
| | | DTKIEVAQFVKDLLLHLKKLFREGQFN |
| 120 | Rhesus monkey IL13 | MALLLTTVIALTCLGGFASPSPVPRSTALKELIEELVNITQ |
| | | NQKAPLCNGSMVWSINLTAGVYCAALESLINVSGCSAIE |
| | | KTQRMLNGFCPHKVSAGQFSSLRVRDTKIEVAQFVKDLL |
| | | VHLKKLFREGREN |
| 121 | Mouse IL13 | MALWVTAVLALACLGGLAAPGPVPRSVSLPLTLKELIEE |
| | | LSNITQDQTPLCNGSMVWSVDLAAGGFCVALDSLTNISN |
| | | CNAIYRTQRILHGLCNRKAPTTVSSLPDTKIEVAHFITKLL |
| | | SYTKQLFRHGPF |
| 122 | Human TSLP | MFPFALLYVLSVSFRKIFILQLVGLVLTYDFTNCDFEKIKA |
| | | AYLSTISKDLITYMSGTKSTEFNNTVSCSNRPHCLTEIQSL |
| | | TFNPTAGCASLAKEMFAMKTKAALAIWCPGYSETQINAT |
| | | QAMKKRRKRKVTTNKCLEQVSQLQGLWRRFNRPLLKQ |
| | | Q |
| 123 | Rhesus monkey | MKSLGQSKKEEVSFRKFFIFQLVGLVLTYDFTNCDFEKIE |
| | TSLP | ADYLRTISKDLITYMSGTKSTDFNNTVSCSNRPHCLTEIQS |
| | | LTFNPTPRCASLAKEMFARKTKATLALWCPGYSETQINA |
| | | TQAMKKRRKRKVTTNKCLEQVSQLLGLWRRFIRTLLKK |
| | | Q |
| 124 | Mouse TSLP | MVLLRSLFILQVLVRMGLTYNFSNCNFTSITKIYCNIIFHD |
| | | LTGDLKGAKFEQIEDCESKPACLLKIEYYTLNPIPGCPSLP |
| | | DKTFARRTREALNDHCPGYPETERNDGTQEMAQEVQNIC |
| | | LNQTSQILRLWYSFMQSPE |
| 125 | Human IL15 | MRISKPHLRSISIQCYLCLLLNSHFLTEAGIHVFILGCFSAG |
| | | LPKTEANWVNVISDLKKIEDLIQSMHIDATLYTESDVHPS |
| | | CKVTAMKCFLLELQVISLESGDASIHDTVENLIILANNSLS |
| | | SNGNVTESGCKECEELEEKNIKEFLQSFVHIVQMFINTS |
| 126 | Rhesus monkey IL15 | MRISKPHLRSVSIQCYLCLLLNSHFLTEAGIHVFILGCFSA |
| | | GLPKTEANWVNVISDLKKIEDLIQSMHIDATLYTESDVHP |
| | | SCKVTAMKCFLLELQVISHESGDTDIHDTVENLIILANNIL |
| | | SSNGNITESGCKECEELEEKNIKEFLQSFVHIVQMFINTS |
| 127 | Mouse IL15 | MKILKPYMRNTSISCYLCFLLNSHFLTEAGIHVFILGCVSV |
| | | GLPKTEANWIDVRYDLEKIESLIQSIHIDTTLYTDSDFHPS |
| | | CKVTAMNCFLLELQVILHEYSNMTLNETVRNVLYLANST |
| | | LSSNKNVAESGCKECEELEEKTFTEFLQSFIRIVQMFINTS |

TABLE 9
Exemplary Fc region sequences

| SEQ ID | | |
|---|---|---|
| NO | Domain or Region | Sequence |
| 40 | AB_TL1A_HL9_002 | TCPPCPAPEAAGAPSVFLFPPKPKDTLMISRTPEVTCVVV |
| | Fc region | DVSHEDPEVKFNWYVDGVEVHNAKTKPREEQYNSTYRV |
| | | VSVLTVLHRDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRWQQ |
| | | GNVFSCSVLHEALKFHYTQKSLSLSPG |
| 41 | AB_TL1A_02_004 | TCPPCPAPEAAGAPSVFLFPPKPKDTLMISRTPEVTCVVV |
| | HL2 Fc region | DVSHEDPEVKFNWYVDGVEVHNAKTKPREEQYNSTYRV |
| | | VSVLTVLHQDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRWQQ |
| | | GNVFSCSVLHEALHSHYTQKSLSLSPG |
| 99 | AB_TL1A_HL9_002_ | TCPPCPAPEAAGAPSVFLFPPKPKDTLMISRTPEVTCVVV |
| | Q318 | DVSHEDPEVKFNWYVDGVEVHNAKTKPREEQYNSTYRV |
| | | VSVLTVLHQDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRWQQ |
| | | GNVFSCSVLHEALKFHYTQKSLSLSPG |
| 100 | AB_TL1A_HL9_002_ | TCPPCPAPEAAGAPSVFLFPPKPKDTLMISRTPEVTCVVV |
| | M435 | DVSHEDPEVKFNWYVDGVEVHNAKTKPREEQYNSTYRV |
| | | VSVLTVLHRDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRWQQ |
| | | GNVFSCSVMHEALKFHYTQKSLSLSPG |
| 101 | AB_TL1A_HL9_002_ | TCPPCPAPEAAGAPSVFLFPPKPKDTLMISRTPEVTCVVV |
| | H440 | DVSHEDPEVKFNWYVDGVEVHNAKTKPREEQYNSTYRV |
| | | VSVLTVLHRDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRWQQ |
| | | GNVFSCSVLHEALHFHYTQKSLSLSPG |
| 102 | AB_TL1A_HL9_002_ | TCPPCPAPEAAGAPSVFLFPPKPKDTLMISRTPEVTCVVV |
| | N441 | DVSHEDPEVKFNWYVDGVEVHNAKTKPREEQYNSTYRV |
| | | VSVLTVLHRDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRWQQ |
| | | GNVFSCSVLHEALKNHYTQKSLSLSPG |
| 42 | RVT-3101 Fc region | TCPPCPAPEAAGAPSVFLFPPKPKDTLMISRTPEVTCVVV |
| | | DVSHEDPEVKFNWYVDGVEVHNAKTKPREEQYNSTYRV |
| | | VSVLTVLHQDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRWQQ |
| | | GNVFSCSVMHEALHNHYTQKSLSLSPG |
| 133 | AB_IL1b_02_003_ | PCPPCPAPEAAGGPSVFLFPPKPKDTLMISRTPEVTCVVV |
| | HL9 Fc | DVSQEDPEVQFNWYVDGVEVHNAKTKPREEQFNSTYRV |
| | | VSVLTVLHRDWLNGKEYKCKVSNKGLPSSIEKTISKAKG |
| | | QPREPQVYTLPPSQEEMTKNQVSLTCLVKGFYPSDIAVE |
| | | WESNGQPENNYKTTPPVLDSDGSFFLYSRLTVDKSRWQE |
| | | GNVFSCSVLHEALKFHYTQKSLSLSLG |
| 134 | AB_IL13_EE_02_001_ | TCPPCPAPEAAGGPSVFLFPPKPKDTLMISRTPEVTCVVV |
| | HL9 Fc | DVSHEDPEVKFNWYVDGVEVHNAKTKPREEQYNSTYRV |
| | | VSVLTVLHRDWLNGKEYKCKVSNKALPAPIEKTISKAKG |
| | | QPREPQVYTLPPSRDELTKNQVSLTCLVKGFYPSDIAVEW |
| | | ESNGQPENNYKTTPPVLDSDGSFFLYSKLTVDKSRWQQG |
| | | NVFSCSVLHEALKFHYTQKSLSLSPG |
| 135 | AB_IL13_EE_02_004_ | TCPPCPAPEAAGGPSVFLFPPKPKDTLMISRTPEVTCVVV |
| | HL9 Fc | DVSHEDPEVKFNWYVDGVEVHNAKTKPREEQYNSTYRV |
| | | VSVLTVLHRDWLNGKEYKCKVSNKALPAPIEKTISKAKG |
| | | QPREPQVYTLPPSRDELTKNQVSLTCLVKGFYPSDIAVEW |
| | | ESNGQPENNYKTTPPVLDSDGSFFLYSKLTVDKSRWQQG |
| | | NVFSCSVLHEALKFHYTQKSLSLSPG |
| 136 | AB_IL13_TSLP_BsAb_ | TCPPCPAPEAAGGPSVFLFPPKPKDTLMISRTPEVTCVVV |
| | ISTF_003_HL9 | DVSHEDPEVKFNWYVDGVEVHNAKTKPREEQYNSTYRV |
| | Fc | VSVLTVLHRDWLNGKEYKCKVSNKALPAPIEKTISKAKG |
| | | QPREPQVYTLPPCREEMTKNQVSLWCLVKGFYPSDIAVE |
| | | WESNGQPENNYKTTPPVLDSDGSFFLYSKLTVDKSRWQQ |
| | | GNVFSCSVLHEALKFHYTQKSLSLSPGK |
| 137 | AB_IL15_AI_02_077_ | TCPPCPAPEAAGGPSVFLFPPKPKDTLMISRTPEVTCVVV |
| | HL9 Fc | DVSHEDPEVKFNWYVDGVEVHNAKTKPREEQYNSTYRV |
| | | VSVLTVLHRDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRWQQ |
| | | GNVFSCSVLHEALKFHYTQKSLSLSPGK |

### EXAMPLES

The following examples are provided to further describe some of the embodiments disclosed herein. The examples are intended to illustrate, not to limit, the disclosed embodiments.

### Example 1. FcRn Binding Affinities of Antibodies Comprising Fe Substitutions

DNA constructs encoding antibodies with a C-terminal fused assay tag were ordered from Gene Fragments. These antibodies were expressed using a prokaryotic in vitro translation system with a disulfide bond enhancer.

Materials utilized in the assay included K Buffer (Assay Buffer) containing 0.02% BSA and 0.002% Tween-20 in PBS, pH 7.4, sourced from Gator Bio, Regen Buffer comprising 10 mM Glycine, pH 1.7, from Gator Bio, and Strep-Tactin XT probes. The protocol involved preparing samples of interest by diluting them in Assay Buffer, then performing analyte dilution series by diluting recombinant purified antigen into assay buffer to the correct target concentration. Samples were loaded into a sample plate and positioned in the plate positions on the BLI device. The assay was conducted in a temperature-controlled environment at 25° C. with shaking conditions set to 1000 rpm, recording data at a frequency of 5 Hz. Protocol cycles included Initial Baseline, Sample Loading, Baseline, Second Baseline, Association, and Dissociation steps. Probes were regenerated after each cycle following a recommended protocol of regeneration and neutralization. This cycle was repeated for every subsequent analyte concentration, typically ranging from 4 to 6 concentrations.

Using Biolayer Interferometry (BLI), AB_TL1A_02_004 was shown to have the binding affinities provided in Table 10 below.

TABLE 10
Binding affinity of AB_TL1A_02_004

| | | | | K_{D} |
|---|---|---|---|---|
| Ab | Kₒₙ | K_{off} | K_{D} (M) | (nM) |
| AB_TL1A_02_004 | 3.798E+05 | 7.381E−04 | 5.169E−09 | 5 |

To perform SPR analysis using the Biacore 8K and Protein A Chip, the chip was equilibrated with Running Buffer (1×HBS-EP+). A solution of the capture ligand (0.35 μg/mL antibody) was prepared in Running Buffer and injected over the chip surface at a flow rate of 10 μL/min for 30 sec until a stable capture level of about 210-470 RU was achieved, followed by washing to remove excess ligand. For sample analysis, analyte samples were prepared in Running Buffer and injected over the chip surface at a flow rate of 30 μL/min for association (180 seconds) and dissociation (3600 seconds). The chip surface was regenerated between sample injections using a regeneration buffer such as 10 mM Gly-HCl, pH 1.5 at a flow rate of 30 μL/min. Sensorgram data was analyzed to determine binding kinetics and affinity, and the steps were repeated for additional samples as needed. The chip was thoroughly washed and regenerated after the experiment, and stored according to manufacturer's recommendations. The data was analyzed using Biacore evaluation software to calculate kinetic parameters for observed interactions.

Using surface plasmon resonance (SPR), AB_TL1A_02_004, AB_TL1A_02_004_HL2, and AB_TL1A_HL9_002 were shown to have the binding affinities provided in Table 11 below.

TABLE 11
Binding affinities of anti-TL1A antibodies

| | | | | K_{D} |
|---|---|---|---|---|
| Ab | Kₒₙ | K_{off} | K_{D} (M) | (pM) |
| AB_TL1A_ | 3.90E+05 | <1.00E−05 | <2.56E−11 | <25.6 |
| 02_004 | 2.13E+05 | <1.00E−05 | <4.69E−11 | <46.9 |
| AB_TL1A_ | 3.82E+05 | <1.00E−05 | <2.62E−11 | <26.2 |
| 02_004_HL2 | | | | |
| AB_TL1A_ | 1.41 + 05 | <1.00E−05 | <7.09E−11 | <70.9 |
| HL9_002 | | | | |

### Example 2. Anti-TL1A Antibodies with Superior Inhibition of TL1A-Mediated Apoptosis

TF-1 cells were seeded at a density of 3E4 cells per well in 96-Well White Opaque Plates, with 50 μper well. A titration of antibody was prepared by 3-fold dilution with a top concentration of 200 nM. The antibody dilutions were then mixed cycloheximide and TL1A to achieve final concentrations of 33 M and 10 nM, respectively, with 25 μL added per well. Plates were subsequently incubated for 6 hours at 37° C. in a humidified incubator with 5% CO2. Following the incubation period, apoptosis was detected using the Caspase-Glo® 3/7 Assay kit, and luminescence was measured using an Envision plate reader. Data analysis was performed using GraphPad Prism 9.0. anti-TL1A antibodies were shown to inhibit TL1A-induced apoptosis with IC50s provided in Table 12 below.

TABLE 12
Neutralization of TL1A by anti-TL1A antibodies

| Ab | IC50 (nM) |
|---|---|
| AB_TL1A_02_004 | 0.82-0.88 |
| | (90-102% max inhibition) |
| AB_TL1A_02_004_ | 0.87 |
| HL2 | (95% max inhibition) |
| AB_TL1A_HL9_002 | 1.10 |
| | (95% max inhibition) |

### Example 3. Anti-TL1A Antibodies with Superior TL1A Receptor Blocking

Plates were coated with 4 g/mL of DR3/Fc or 1 g/mL of DcR3/Fc by adding 100 μL per well, and were incubated overnight at 4° C. 1×PBST was added at 200 μL per well, and after a 10-second incubation, the liquid was poured out and the plate was pat dried on blotting paper. This process was repeated three times. The plates were then blocked with 2% BSA in 1×PBST at 200 μL per well for 2 hours at room temperature (RT), followed by a similar washing step. Antibodies were diluted, and test antibodies were serially diluted and pre-incubated with biotinylated recombinant human TL1A at 3.5 nM for 30 minutes. This mixture was then added to the DR3/Fc or DcR3/Fc coated wells and incubated at RT for 1 hour. Further washing steps were performed, followed by the addition of a secondary antibody at 100 μL per well, which was incubated at RT for 0.5 hours. Additional washing steps were conducted, and TMB was added to each well at 100 μL and incubated at RT for 15 minutes. The reaction was stopped with 100 μL of 2M HCl stop solution per well, and the plates were read with a microplate reader at 450 nm. For buffer preparation, coating buffer consisted of 1×PBS, washing buffer was 0.05% tween-20 in 1×PBS, blocking buffer was 1% BSA in 1×PBST, and both test antibody dilution buffer and secondary antibody dilution buffer were prepared using blocking buffer.

Anti-TL1A antibodies were shown to neutralize binding of TL1A to DR3/Fc or DcR3/Fc with IC50s as provided in Table 13 below.

TABLE 13
Blocking of TL1A receptor binding by anti-TL1A antibodies

| | DR3/Fc IC50 | DcR3/Fc IC50 |
|---|---|---|
| Ab | (nM) | (nM) |
| AB_TL1A_02_004 | 0.92-1.01 | 1.20-1.51 |
| | (96.9-98.1 max | (98.9 max |
| | inhibition) | inhibition) |
| AB_TL1A_02_004_HL2 | 0.58 | 1.30 |
| | (96.7% max | (99.4% max |
| | inhibition) | inhibition) |
| AB_TL1A_HL9_002 | 1.22 | 1.55 |
| | (97.8% max | (98.5% max |
| | inhibition) | inhibition) |

### Example 4. Anti-TL1A Antibodies with Low Immunogenicity

Immunogenicity of anti-TL1A antibody candidates AB_TL1A_02_004_HL2 and AB_TL1A_HL9_002 was predicted using AlphaMHC v4.0.1(WeComput Technology), and is shown in Table 14A below. AB_TL1A_02_004_HL2 is predicted to have comparable immunogenicity, and AB_TL1A_HL9_002 is predicted to have low immunogenicity, compared to that of comparator antibody RVT-3101.

TABLE 14A
Immunogenicity of anti-TL1A antibodies

| Ab | Immunogenicity Score |
|---|---|
| AB_TL1A_02_004_ | 0.22 |
| HL2 | |
| AB_TL1A_HL9_002 | 0.15 |

Immunogenicity of antibodies was predicted using an AlphaMHC v4.1.0 (WeComput Technology). Antibodies comprising a domain capable of binding TL1A were assessed. Immunogenicity data is shown in Table 14B below. Antibodies with the Q311R, M428L, H433K, and N434F substitutions had lower immunogenicity or substantially similar immunogenicity to RVT-3101.

TABLE 14B
Immunogenicity of anti-TL1A antibodies

| | HLE | Immunogenicity |
|---|---|---|
| Antibody | Mutation † | Score |
| Target | — | <0.20 |
| RVT-3101 | — | 0.27 |
| AB_TL1A_02_ | Q311R, M428L, | 0.32 |
| 004_HL9 | H433K, N434F | |
| AB_TL1A_ | Q311R, M428L, | 0.15 |
| HL9_002 | H433K, N434F | |
| AB_TL1A_02_ | H-M252Y, H-S254T, | 0.25 |
| 004_HL1 | H-T256E | |
| AB_TL1A_02_ | H-M428L, H-N434S | 0.22 |
| 004_HL2 | | |

Immunogenicity of antibodies disclosed herein is tested by cytokine release assay with PBMCs and/or whole blood from healthy donors. One or more antibodies produce comparable or lower levels of pro-inflammatory cytokines compared to comparator antibodies (e.g., RVT-3101).

### Example 5. Antibodies with Increased FcRn Affinity

Binding affinities of anti-TL1A antibody candidates AB_TL1A_02_004_HL2 and AB_TL1A_HL9_002 for FcRn was measured by SPR. FcRn binding kinetics at pH 6.0 and pH 7.4 were measured using a Biacore® 8K instrument (Cytiva) with a CM5 chip. Binding affinities are presented in Table 15 below.

TABLE 15
Binding affinities of anti-TL1A antibodies for FcRn

| Ab | K_{D} at pH 6.0 | K_{D} at pH 7.4 |
|---|---|---|
| AB_TL1A_02_004_HL2 | 1.59E−07 M | No or weak |
| | 1.90E−07 M | binding |
| AB_TL1A_HL9_002 | 4.53E−08 M | No or weak |
| | 5.09E−08 M | binding |

To perform SPR analysis using a Biacore 8K and CM5 chip, the TL1A antibody was immobilized at 10 g/mL concentration and binding to the human FcRn analyte at various concentrations (2-fold dilutions from 6000 nM) was tested. The 1×PBST running buffer was prepared at both pH 6.0 and pH 7.4. Kinetic measurements were obtained for association and dissociation times of 60 and 90 seconds, respectively, at a flow rate of 30 μL/min. The CM5 chips were regenerated with 1×PBS (pH 7.4). Kinetic parameters for the concentration series at both pHs were fit to a steady state affinity model and averaged using the Biacore Evaluation Software. Each concentration series was fit independently to obtain the average rates and binding affinities. The half-life was compared to that of the commercial antibody, RVT-3101, which is known to have a 3-week half-life. Binding affinities for the following TL1A-binding antibodies are provided in Table 16 below.

TABLE 16
Binding affinities of anti-TL1A antibodies for FcRn

| | | Human | Human | Human |
|---|---|---|---|---|
| | | FcRn | FcRn | FcRn |
| | HLE | pH 6.0 | pH 6.0 | pH 7.4 |
| Antibody | Mutation † | (M) | (FI)* | (M) |
| RVT-3101 | — | 2.44E−06 | 1.0 | No or weak |
| | | | | binding |
| | | 2.45E−06 | 1.0 | No or weak |
| | | | | binding |
| | | 2.46E−06 | 1.0 | No or weak |
| | | | | binding |
| AB_TL1A_ | — | 2.43E−06 | 1.0 | No or weak |
| 02_004 | | | | binding |
| | | 2.24E−06 | 1.1 | No or weak |
| | | | | binding |
| AB_TL1A_ | Q311R, | 4.61E−07 | 5.3 | No or weak |
| 02_004_HL3 | M428L | | | binding |
| AB_TL1A_02_ | H433K, | 1.32E−07 | 18.5 | >1.50E+6, |
| 004 HL4 | N434F | | | No or weak |
| | | | | binding |
| AB_TL1A_ | Q311R, | 3.60E−08 | 67.8 | >1.50E+6, |
| 02_004_HL9 | M428L, | | | No or weak |
| | H433K, | | | binding |
| | N434F | 4.33E−08 | 56.4 | No or weak |
| | | | | binding |
| AB_TL1A_ | Q311R, | 4.53E−08 | 53.9 | No or weak |
| HL9_002 | M428L, | | | binding |
| | H433K, | 5.09E−08 | 48.0 | No or weak |
| | N434F | | | binding |
| AB_TL1A_ | M428L, | 6.08E−08 | 40.7 | No or weak |
| HL9_002_ | H433K, | | | binding |
| Q318 | N434F | | | |
| AB_TL1A_ | Q311R, | 8.47E−08 | 28.8 | No or weak |
| HL9_002_ | H433K, | | | binding |
| M435 | N434F | | | |
| AB_TL1A_ | Q311R, | 5.77E−08 | 42.3 | No or weak |
| HL9_002_ | M428L, | | | binding |
| H440 | N434F | | | |
| AB_TL1A_ | Q311R, | 2.88E−07 | 8.5 | No or weak |
| HL9_002_ | M428L, | | | binding |
| N441 | H433K | | | |

*Fold Increase (FI) relative to RVT-3101 (Roche)

Antibodies comprising the Q311R and M428L substitutions (TL1A_02_004_HL3) or the H433K and N434F substitutions (AB_TL1A_02_004_HL4 containing) resulted in a 5.3-fold and 18.5-fold increase, respectively, in FcRn binding affinity at pH 6.0 compared to RVT-3101. Antibody constructs comprising all four substitutions (Q311R, M428L, H433K, and N434F) demonstrated a synergistic effect, providing a 48.0-fold to 67.8-fold increase in FcRn binding affinity at pH 6.0 compared to RVT-3101. Removing the Q311R or H433K mutations had minimal impact on the FcRn binding affinity; such constructs (AB_TL1A_HL9_002_Q318 and AB_TL1A_HL9_002_H440) maintained FcRn binding affinities at pH 6.0 that were 40.7-fold and 42.3-fold higher, respectively, than RVT-3101. Reverting the M428L mutation (e.g., AB_TL1A_HL9_002_M435) had an intermediate impact on the FcRn binding affinity (28.8-fold higher than RVT-3101 at pH 6.0). Removing the N434F mutation (e.g., AB_TL1A_HL9_002_N441) had the largest impact on FcRn binding affinity, which was only 8.5-fold higher than RVT-3101 at pH 6.0.

Collectively, these data indicate that the N434F substitution contributes strongly to increased FcRn binding affinity. The Q311R, M428L and H433K mutations provide a more moderate contribution to increased FcRn binding affinity. However, constructs with the N434F mutation and any two of the Q311R, M428L, and H433K substitutions provide increased binding affinity compared to the combination of Q311R and M428L, or the combination of H433K and N434F, alone.

Antibodies comprising a binding domain for any one of IL1β, IL13, and IL15 were modified to include the Q311R, M428L, H433K, and N434F substitutions. A bispecific antibody comprising a binding domain for TSLP and a binding domain for IL13 was also modified to include the Q311R, M428L, H433K, and N434F substitutions. SPR analysis was performed using a Bioacore 8K instrument and a CM5 with the antibody immobilized at 10 g/mL concentration, as above. Binding affinities for the antibodies are provided in Table 17 below.

TABLE 17
Binding affinities of anti-TL1A, anti- IL1B, anti-IL13,
anti-IL13 × TSLP and anti-IL15 antibodies

| | | Human | Human | Human |
|---|---|---|---|---|
| | HLE | FcRn | FcRn | FcRn |
| | Muta- | pH 6.0 | pH 6.0 | pH 7.4 |
| Antibody | tion † | (M) | (FI)* | (M) |
| Rituximab † | — | 2.13E−06 | 1.0 | No/weak |
| | | | | binding |
| RVT-3101¹ | — | 2.44E−06 | 0.9 | No/weak |
| | | | | binding |
| | | 2.45E−06 | 0.9 | No/weak |
| | | | | binding |
| | | 2.46E−06 | 0.9 | No/weak |
| | | | | binding |
| AB_TL1A_ | — | 2.43E−06 | 0.9 | No/weak |
| 02_004 | | | | binding |
| | | 2.24E−06 | 1.0 | No/weak |
| | | | | binding |
| AB_TL1A_02_ | M252Y, | 2.18E−07 | 9.8 | No/weak |
| 004_HL1 | S254T, | | | binding |
| | T256E | | | |
| AB_TL1A_02_ | M428L, | 1.59E−07 | 13.4 | No/weak |
| 004_HL2 | N434S | | | binding |
| | | 1.90E−07 | 11.2 | No/weak |
| | | | | binding |
| AB_TL1A_02_ | Q311R, | 3.60E−08 | 59.2 | No/weak |
| 004_HL9 | M428L, | | | binding^{‡} |
| | H433K, | 4.33E−08 | 49.2 | No/weak |
| | N434F | | | binding |
| AB_TL1A_ | Q311R, | 4.53E−08 | 47.0 | No/weak |
| HL9_002 | M428L, | | | binding |
| | H433K, | 5.09E−08 | 41.8 | No/weak |
| | N434F | | | binding |
| AVTX-009 | — | 2.19E−06 | 1.0 | No/weak |
| (Avalo)² | | | | binding |
| AB_IL1b_ | M428L, | 1.57E−07 | 13.6 | No/weak |
| 02_003 | N434S | | | binding |
| AB_IL1b_02_ | Q311R, | 4.23E−08 | 50.4 | No/weak |
| 003_HL9 | M428L, | | | binding |
| | H433K, | | | |
| | N434F | | | |
| APG-777 | M252Y, | 2.02E−07 | 10.5 | No/weak |
| (Apogee)³ | S254T, | | | binding |
| | T256E | | | |
| AB_IL13_ | M252Y, | 2.07E−07 | 10.3 | No/weak |
| 02_001 | S254T, | | | binding |
| | T256E | | | |
| AB_IL13_ | M252Y, | 2.11E−07 | 10.1 | No/weak |
| 02_004 | S254T, | | | binding |
| | T256E | | | |
| AB_IL13_ | Q311R, | 4.48E−08 | 47.5 | No/weak |
| 02_001_HL9 | M428L, | | | binding |
| | H433K, | | | |
| | N434F | | | |
| AB_IL13_02_ | Q311R, | 4.64E−08 | 45.9 | No/weak |
| 004_HL9 | M428L, | | | binding |
| | H433K, | | | |
| | N434F | | | |
| APG-777 × | M428L, | NT | NA | NT |
| AIO-001 | N434S | | | |
| (Aiolos/GSK)⁴ | | | | |
| AB_IL13_TSLP_ | M428L, | 1.03E−07 | 20.7 | No/weak |
| BsAb_ISTF_003 | N434S | | | binding |
| AB_IL13_TSLP_ | Q311R, | 4.39E−08 | 48.5 | No/weak |
| BsAb_ISTF_003_ | M428L, | | | binding |
| HL9 | H433K, | | | |
| | N434F | | | |
| CALY-002 | — | 1.95E−06 | 1.1 | No/weak |
| (Calypso/ | | | | binding |
| Novartis)⁵ | | | | |
| AB_IL15_AI_ | M428L, | NT | NA | NT |
| 02_077 | N434S | | | |
| AB_IL15_AI_ | Q311R, | 5.59E−08 | 38.1 | No/weak |
| 02_077_HL9 | M428L, | | | binding |
| | H433K, | | | |
| | N434F | | | |

*Fold Increase (FI) relative to Rituximab
† Average results from multiple experiments
^{‡}<1.50E+6
¹anti-TL1A comparator antibody;
²anti-IL1ß comparator antibody;
³anti-IL13 comparator antibody;
⁴Bispecific anti-IL13 × TSLP comparator antibody;
⁵anti-IL15 comparator antibody

Antibodies comprising the Q311R, M428L, H43K, and N434F substitutions demonstrated higher FcRn binding affinity compared to commercial antibody rituximab, which does not comprise any of these substitutions. The effect of these substitutions on FcRn binding affinity was consistent for antibodies with different antigen specificities. FcRn binding affinity at pH 6.0 was increased by 38.1-fold to 59.2-fold, compared to rituximab, for antibodies comprising a binding domain for any one of IL1β, IL13, and IL15, as well as for a bispecific antibody comprising a binding domain for TSLP and a binding domain for IL13. The combination of Q311R, M428L, H433K, and N434F substitutions compared favorably to other combinations of substitutions. For example, FcRn binding affinity for antibodies comprising the Q311R, M428L, H433K, and N434F substitutions was consistently higher than for antibodies comprising the M428L and N434S substitutions (also called “LS” substitutions) or the M252Y, S254T, and T256E substitutions (also called “YTE” substitutions). The FcRn binding affinity of antibodies with the LS substitutions were only 11.2-fold to 20.7-fold higher than rituximab, and the FcRn binding affinity of antibodies with the YTE substitutions were only 9.8-fold to 10.5-fold higher than rituximab, whereas the FcRn binding affinity of antibodies with the Q311R, M428L, H433K, and N434F substitutions were 38.1-fold to 59.2-fold higher than rituximab. These results indicate that the combination of the Q311R, M428L, H433K, and N434F substitutions can be used as a platform technology for increasing the FcRn binding affinity of therapeutic antibodies.

### Example 6. Antibodies with Improved Pharmacokinetic Properties

Pharmacokinetics of anti-TL1A antibody candidates AB_TL1A_02_004_HL2 and AB_TL1A_HL9_002 were predicted using PK-Sim and MOBI, which are physiologically based pharmacokinetic (PBPK) modeling software, based on clinical data from ongoing trials. This in silico approach allows determination of pharmacokinetics (PK) and translation of in vitro data for first-in-human (FIH) starting dose selection. As shown in Table 18 below, both AB_TL1A_02_004_HL2 and AB_TL1A_HL9_002 are predicted to have a longer dosing interval (once every 6 months, and once every 12 months, respectively) compared to RVT-3101, which is dosed once monthly.

TABLE 18
Predicted dosing interval of anti-TL1A antibodies

| | AB_TL1A_ | AB_TL1A_ |
|---|---|---|
| | 02_004HL2 | HL9_002 |
| Dosing Interval | Q6M | Q12M |

### Example 7. Production Titers and Recovery Yields

Production titers and recovery yields are provided in Tables 19A and 19B below.

TABLE 19A
Production titers and recovery yields

| | | | | | SDS-PAGE | Endotoxin |
|---|---|---|---|---|---|---|
| | Yield | SEC 214 | SEC 280 | SDS-PAGE | (Non- | level |
| Ab | (mg) | nm | nm | (Reducing) | reducing) | (EU/mg) |
| AB_TL1A_02_004 | 7.35 | 93.342% | 94.025% | >95% | >95% | 0-1 |

| | | | | Non- | |
|---|---|---|---|---|---|
| | | | | Reducing | |
| | Titer | Yield | SEC-HPLC | Caliper | Endotoxin |
| Ab | (mg/mL) | (mg) | (% Main) | (% Main) | (EU/mg) |
| AB_TL1A_02_004 | 1.20 | 182.0 | >95% | >95% | 0-1 |
| AB_TL1A_02_004_HL2 | 2.12 | 306.4 | >95% | >95% | 0-1 |
| AB_TLIA_HL9_002 | 1.89 | 228.2 | >95% | >95% | 0-1 |

Production was carried out in 200 ml culture volume in CHO. Purity was checked using non-reducing Caliper and SEC analysis.

TABLE 19B
Production titers and recovery yields

| | HL | Titer | Yield |
|---|---|---|---|
| Asset | Mutation | (g/L) | (mg/L) |
| Target | — | 0.5-2.0 | 250-1000 |
| RVT-3101 | — | 2.0 | 1439 |
| (Roche)¹ | | | |
| AB_TL1A_ | Q311R, | 2.4 | 675 |
| 02_004_HL3 | M428L | | |
| AB_TL1A_ | H433K, | 2.4 | 489 |
| 02_004_HL4 | N434F | | |
| AB_TL1A_ | Q311R, | 1.9 | 295 |
| 02_004_HL9 | M428L, | | |
| | H433K, | | |
| | N434F | | |
| AB_TL1A_ | Q311R, | 1.5 | 785 |
| HL9_002 | M428L, | | |
| | H433K, | | |
| | N434F | | |
| AB_TL1A_ | M428L, | 2.2 | 1006 |
| HL9_002_ | H433K, | | |
| Q318 | N434F | | |
| AB_TL1A_ | Q311R, | 1.7 | 699 |
| HL9_002_ | H433K, | | |
| M435 | N434F | | |
| AB_TL1A_ | Q311R, | 1.7 | 458 |
| HL9_002_ | M428L, | | |
| H440 | N434F | | |
| AB_TL1A_ | Q311R, | 2.2 | 964 |
| HL9_002_ | M428L, | | |
| N441 | H433K | | |
| AVTX-009 | — | 2.2 | 1144 |
| (Avalo)² | | | |
| APG-777 | H-M252Y, | 2.2 | 1080 |
| (Apogee)³ | H-S254T, | | |
| | H-T256E | | |
| APG-777 | H-M428L, | 1.2 | 266 |
| (Apogee) × | H-N434S | | |
| AIO-001 | | | |
| (Aiolos/ | | | |
| GSK)⁴ | | | |
| AB_TL1A_ | H-M252Y, | 2.6 | 844 |
| 02_004_HL1 | H-S254T, | | |
| | H-T256E | | |
| AB_TL1A_ | H-M428L, | 2.1 | 645 |
| 02_004_HL2 | H-N434S | | |
| AB_IL1b_ | H-M428L, | 1.6 | 1012 |
| 02_003 | H-N434S | | |
| AB_IL13_ | H-M252Y, | 1.8 | 991 |
| 02_001 | H-S254T, | | |
| | H-T256E | | |
| AB_IL13_ | H-M252Y, | 1.5 | 714 |
| 02_004 | H-S254T, | | |
| | H-T256E | | |
| AB_IL13_ | H-M428L, | 1.0 | 238 |
| TSLP_BsAb_ | H-N434S | | |
| ISTF_003 | | | |
| AB_TL1A_ | H-Q311R, | 1.9 | 295 |
| 02_004_HL9 | H-M428L, | | |
| | H-H433K, | | |
| | H-N434F | | |
| AB_IL1b_ | H-Q311R, | 0.9 | 437 |
| 02_003_HL9 | H-M428L, | | |
| | H-H433K, | | |
| | H-N434F | | |
| AB_IL13_ | H-Q311R, | 1.4 | 908 |
| 02_001_HL9 | H-M428L, | | |
| | H-H433K, | | |
| | H-N434F | | |
| AB_IL13_ | H-Q311R, | 1.5 | 842 |
| 02_004_HL9 | H-M428L, | | |
| | H-H433K, | | |
| | H-N434F | | |
| AB_IL13_ | H-Q311R, | 0.9 | 93 |
| TSLP_BsAb_ | H-M428L, | | |
| ISTF_003_ | H-H433K, | | |
| HL9 | H-N434F | | |
| IL15 REF⁵ | — | NT | 161 |
| AB-IL15-AI- | H-M428L, | NT | 38 |
| 02-077 | H-N434S | | |
| AB_IL15_ | H-Q311R, | 0.07 | 31 |
| AI_02_077_ | H-M428L, | | |
| HL9 | H-H433K, | | |
| | H-N434F | | |

¹anti-TL1A comparator antibody;
²anti-IL1ß comparator antibody;
³anti-IL13 comparator antibody;
⁴Bispecific anti-IL13 × TSLP comparator antibody;
⁵anti-IL15 comparator antibody

Those skilled in the art will appreciate that numerous changes and modifications can be made to the preferred embodiments disclosed herein and that such changes and modifications can be made without departing from the spirit of the invention. It is, therefore, intended that the appended claims cover all such equivalent variations as fall within the true spirit and scope of the invention.

What is claimed:

1. A polypeptide comprising a variant IgG Fc region, wherein the variant IgG Fc region comprises amino acid substitutions at:
a) position Q311 and/or M428; and
b) position H433 and/or N434;
relative to a wild-type IgG Fc region, wherein numbering is according to the EU index as in Kabat.

2. The polypeptide of claim 1, wherein the variant IgG Fc region comprises amino acid substitutions at:
a) Q311 and H433;
b) M428 and N434;
c) Q311 and N434;
d) M428 and H433;
e) Q311, M428, and H433;
f) Q311, M428, and N434;
g) Q311, H433, and N434; or
h) M428, H433, and N434.

3. The polypeptide of claim 1 or claim 2, wherein the variant IgG Fc region comprises amino acid substitutions at Q311, M428, and N434.

4. The polypeptide of any one of claims 1-3, wherein the variant IgG Fc region comprises amino acid substitutions at M428, and H433, and N434.

5. The polypeptide of any one of claims 1-4, wherein the variant IgG Fc region comprises amino acid substitutions at Q311, M428, H433, and N434.

6. The polypeptide of any one of claims 1-5, wherein the amino acid substitutions comprise:
a) Q311R and H433K;
b) M428L and N434F;
c) Q311R and N434F;
d) M428L and H433K;
e) Q311R, M428L, and H433K;
f) Q311R, M428L, and N434F;
g) Q311R, H433K, and N434F; or
h) M428L, H433K, and N434F.

7. The polypeptide of any one of claims 1-6, wherein the amino acid substitutions comprise Q311R, M428L, and N434F.

8. The polypeptide of any one of claims 1-7, wherein the amino acid substitutions comprise M428L, H433K, and N434F.

9. The polypeptide of any one of claims 1-8, wherein the amino acid substitutions comprise Q311R, M428L, H433K, and N434F.

10. The polypeptide of any one of claims 1-9, wherein the variant IgG Fc region further comprises one or more amino acid substitutions selected from the group consisting of L234A, L235A, and G237A.

11. The polypeptide of any one of claims 1-10, wherein the variant IgG Fc region is an IgG1 Fc region.

12. The polypeptide of any one of claims 1-11, wherein the variant IgG Fc region is an IgG4 Fc region.

13. The polypeptide of any one of claims 1-12, wherein the variant IgG Fc region is a human IgG Fc region.

14. The polypeptide of any one of claims 1-13, wherein the polypeptide comprising a variant IgG Fc region has increased affinity for FcRn compared to an IgG having a wild-type IgG Fc region.

15. The polypeptide of any one of claims 1-14, wherein the polypeptide comprising a variant IgG Fc region has increased affinity for FcRn compared to an IgG having a reference IgG Fc region comprising the amino acid sequence of SEQ ID NO: 42, 107, 108, 109, or 110.

16. The polypeptide of any one of claims 1-15, wherein the polypeptide comprising a variant IgG Fc region has an increased half-life compared to the half-life of an IgG having a wild-type IgG Fc region.

17. The polypeptide of any one of claims 1-16, wherein the polypeptide comprising a variant IgG Fc region has an increased half-life compared to the half-life of an IgG having a reference IgG Fc region comprising the amino acid sequence of SEQ ID NO: 42, 107, 108, 109, or 110.

18. The polypeptide of any one of claims 1-17 wherein the variant IgG Fc region comprises the amino acid sequence of any one of SEQ ID NOs: 40, 41, 99-102, or 133-137.

19. The polypeptide of any one of claims 1-18, wherein the polypeptide comprises an antibody or antigen-binding fragment thereof.

20. The polypeptide of any one of claims 1-19, wherein the polypeptide is an antibody.

21. The polypeptide of claim 19 or claim 20, wherein the antibody is a human antibody, a humanized antibody, or monoclonal antibody.

22. The polypeptide of any one of claims 19-21, wherein the antibody comprises a heavy chain constant region comprising any one of SEQ ID NOs: 33-35, 95-98, and 128-132.

23. The polypeptide of any one of claims 19-22, wherein the antibody or antigen-binding fragment thereof binds tumor necrosis factor-like ligand 1A (TL1A), IL13, IL13, IL15, or TSLP.

24. The polypeptide of any one of claims 19-23, wherein the antibody comprises two or more antigen binding regions.

25. The polypeptide of claim 24, wherein one of the two or more binding regions binds IL13 or TSLP.

26. The polypeptide of claim 25, wherein the antibody comprises a first binding region that binds IL13 and a second binding region that binds TSLP.

27. The polypeptide of any one of claims 1-26, for use as a medicament.

28. An immunoconjugate comprising the polypeptide of any one of claims 1-26, and a therapeutic agent, wherein the polypeptide is linked to the therapeutic agent.

29. A pharmaceutical composition comprising the polypeptide of any one of claims 1-26, or the immunoconjugate of claim 28, and a pharmaceutically acceptable carrier, diluent or excipient.

30. A nucleic acid molecule encoding the polypeptide of any one of claims 1-27.

31. An expression vector comprising the nucleic acid molecule of claim 30.

32. A recombinant cell comprising the polypeptide of any one of claims 1-26, the nucleic acid molecule of claim 30, or the vector of claim 31.

33. A method of producing the polypeptide of any one of claims 1-26, wherein the polypeptide is a recombinant polypeptide comprising a variant Fc, and wherein the recombinant polypeptide is produced using a recombinant vector in a recombinant cell.

34. An antibody or antigen-binding fragment thereof that binds tumor necrosis factor-like ligand 1A (TL1A), wherein the antibody or antigen-binding fragment thereof comprises:
a) a heavy chain variable region (VH) comprising an amino acid sequence with at least 90% sequence identity to SEQ ID NO: 7 and a light chain variable region (VL) comprising an amino acid sequence with at least 90% sequence identity to SEQ ID NO: 8;
b) a VH comprising an amino acid sequence with at least 90% sequence identity to SEQ ID NO: 13 and a VL comprising an amino acid sequence with at least 90% sequence identity to SEQ ID NO: 14;
c) a VH comprising an amino acid sequence with at least 90% sequence identity to SEQ ID NO: 7 and a VL comprising an amino acid sequence with at least 90% sequence identity to SEQ ID NO: 18;
d) a VH comprising an amino acid sequence with at least 90% sequence identity to SEQ ID NO: 7 and a VL comprising an amino acid sequence with at least 90% sequence identity to SEQ ID NO: 20;
e) a VH comprising an amino acid sequence with at least 90% sequence identity to SEQ ID NO: 23 and a VL comprising an amino acid sequence with at least 90% sequence identity to SEQ ID NO: 20; or
f) a VH comprising an amino acid sequence with at least 90% sequence identity to SEQ ID NO: 28 and a VL comprising an amino acid sequence with at least 90% sequence identity to SEQ ID NO: 20.

35. The antibody or antigen-binding fragment thereof of claim 34, wherein the antibody or antigen-binding fragment thereof comprises:
a) a heavy chain complementarity determining region 1 (HCDR1) having at least 90% sequence identity with SEQ ID NO: 1, a HCDR2 having at least 90% sequence identity with SEQ ID NO: 2, a HCDR3 having at least 90% sequence identity with SEQ ID NO: 3, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6;
b) a HCDR1 having at least 90% sequence identity with SEQ ID NO: 1, a HCDR2 having at least 90% sequence identity with SEQ ID NO: 11, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 5, and a LCDR3 having at least 90% sequence identity with SEQ ID NO: 12;
c) a HCDR1 having at least 90% sequence identity with SEQ ID NO: 1, a HCDR2 having at least 90% sequence identity with SEQ ID NO: 2, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 6;
d) a HCDR1 having at least 90% sequence identity with SEQ ID NO: 1, a HCDR2 having at least 90% sequence identity with SEQ ID NO: 2, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 12;
e) a HCDR1 having at least 90% sequence identity with SEQ ID NO: 1, a HCDR2 having at least 90% sequence identity with SEQ ID NO: 22, a HCDR3 having at least 90% sequence identity with SEQ ID NO: 3, a LCDR1 having at least 90% sequence identity with SEQ ID NO: 4, a LCDR2 having at least 90% sequence identity with SEQ ID NO: 17, and a LCDR3 having at least 90% sequence identity with SEQ ID NO: 12;
f) a HCDR1 having at least 90% sequence identity with SEQ ID NO: 25, a HCDR2 having at least 90% sequence identity with SEQ ID NO: 2, a HCDR3 having at least 90% sequence identity with SEQ ID NO: 3, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6; or
g) a HCDR1 having at least 90% sequence identity with SEQ ID NO: 25, a HCDR2 having at least 90% sequence identity with SEQ ID NO: 2, a HCDR3 having at least 90% sequence identity with SEQ ID NO: 27, a light chain complementarity determining region 1 (LCDR1) having at least 90% sequence identity with SEQ ID NO: 4, a LCDR2 having at least 90% sequence identity with SEQ ID NO: 5, and a LCDR3 having at least 90% sequence identity with SEQ ID NO: 6.

36. The antibody or antigen-binding fragment thereof of claim 34 or claim 35, comprising:
a) a heavy chain variable region (VH) comprising the amino acid sequence of SEQ ID NO: 7 and a light chain variable region (VL) comprising the amino acid sequence of SEQ ID NO: 8;
b) a VH comprising the amino acid sequence of SEQ ID NO: 13 and a VL comprising the amino acid sequence of SEQ ID NO: 14;
c) a VH comprising the amino acid sequence of SEQ ID NO: 7 and a VL comprising the amino acid sequence of SEQ ID NO: 18;
d) a VH comprising the amino acid sequence of SEQ ID NO: 7 and a VL comprising the amino acid sequence of SEQ ID NO: 20;
e) a VH comprising the amino acid sequence of SEQ ID NO: 23 and a VL comprising the amino acid sequence of SEQ ID NO: 20; or
f) a VH comprising the amino acid sequence of SEQ ID NO: 28 and a VL comprising the amino acid sequence of SEQ ID NO: 20.

37. An antibody or antigen-binding fragment thereof that binds interleukin 1β (IL1β), wherein the antibody or antigen-binding fragment thereof comprises a heavy chain variable region (VH) comprising an amino acid sequence with at least 90% sequence identity to SEQ ID NO: 49 and a light chain variable region (VL) comprising an amino acid sequence with at least 90% sequence identity to SEQ ID NO: 50.

38. The antibody or antigen-binding fragment thereof of claim 37, wherein the antibody or antigen-binding fragment thereof comprises: a heavy chain complementarity determining region 1 (HCDR1) having at least 90% sequence identity with SEQ ID NO: 43, a HCDR2 having at least 90% sequence identity with SEQ ID NO: 44, a HCDR3 having at least 90% sequence identity with SEQ ID NO: 45, a light chain complementarity determining region 1 (LCDR1) having at least 90% sequence identity with SEQ ID NO: 46, a LCDR2 having at least 90% sequence identity with SEQ ID NO: 47, and a LCDR3 having at least 90% sequence identity with SEQ ID NO: 48.

39. The antibody or antigen-binding fragment thereof of claim 37 or claim 38, comprising: a heavy chain variable region (VH) comprising the amino acid sequence of SEQ ID NO: 49 and a light chain variable region (VL) comprising the amino acid sequence of SEQ ID NO: 50.

40. An antibody or antigen-binding fragment thereof that binds interleukin 13 (IL13), wherein the antibody or antigen-binding fragment thereof comprises:
a) a heavy chain variable region (VH) comprising an amino acid sequence with at least 90% sequence identity to SEQ ID NO: 59 and a light chain variable region (VL) comprising an amino acid sequence with at least 90% sequence identity to SEQ ID NO: 60;
b) a VH comprising an amino acid sequence with at least 90% sequence identity to SEQ ID NO: 64 and a VL comprising an amino acid sequence with at least 90% sequence identity to SEQ ID NO: 60; or
c) a VH comprising an amino acid sequence with at least 90% sequence identity to SEQ ID NO: 86 and a VL comprising an amino acid sequence with at least 90% sequence identity to SEQ ID NO: 87.

41. The antibody or antigen-binding fragment thereof of claim 40, wherein the antibody or antigen-binding fragment thereof comprises:
a) a heavy chain complementarity determining region 1 (HCDR1) having at least 90% sequence identity with SEQ ID NO: 53, a HCDR2 having at least 90% sequence identity with SEQ ID NO: 54, a HCDR3 having at least 90% sequence identity with SEQ ID NO: 55, a light chain complementarity determining region 1 (LCDR1) having at least 90% sequence identity with SEQ ID NO: 56, a LCDR2 having at least 90% sequence identity with SEQ ID NO: 57, and a LCDR3 having at least 90% sequence identity with SEQ ID NO: 58;
b) a HCDR1 having at least 90% sequence identity with SEQ ID NO: 53, a HCDR2 having at least 90% sequence identity with SEQ ID NO: 54, a HCDR3 having at least 90% sequence identity with SEQ ID NO: 63, a LCDR1 having at least 90% sequence identity with SEQ ID NO: 56, a LCDR2 having at least 90% sequence identity with SEQ ID NO: 57, and a LCDR3 having at least 90% sequence identity with SEQ ID NO: 58; or
c) a HCDR1 having at least 90% sequence identity with SEQ ID NO: 53, a HCDR2 having at least 90% sequence identity with SEQ ID NO: 54, a HCDR3 having at least 90% sequence identity with SEQ ID NO: 82, a LCDR1 having at least 90% sequence identity with SEQ ID NO: 56, a LCDR2 having at least 90% sequence identity with SEQ ID NO: 57, and a LCDR3 having at least 90% sequence identity with SEQ ID NO: 58.

42. The antibody or antigen-binding fragment thereof of claim 40 or claim 41, comprising:
a) a heavy chain variable region (VH) comprising the amino acid sequence of SEQ ID NO: 59 and a light chain variable region (VL) comprising the amino acid sequence of SEQ ID NO: 60;
b) a VH comprising the amino acid sequence of SEQ ID NO: 64 and a VL comprising the amino acid sequence of SEQ ID NO: 60; or
c) a VH comprising the amino acid sequence of SEQ ID NO: 86 and a VL comprising the amino acid sequence of SEQ ID NO: 87.

43. An antibody or antigen-binding fragment thereof that binds interleukin 15 (IL15), wherein the antibody or antigen-binding fragment thereof comprises a heavy chain variable region (VH) comprising an amino acid sequence with at least 90% sequence identity to SEQ ID NO: 72 and a light chain variable region (VL) comprising an amino acid sequence with at least 90% sequence identity to SEQ ID NO: 73.

44. The antibody or antigen-binding fragment thereof of claim 43, wherein the antibody or antigen-binding fragment thereof comprises: a heavy chain complementarity determining region 1 (HCDR1) having at least 90% sequence identity with SEQ ID NO: 66, a HCDR2 having at least 90% sequence identity with SEQ ID NO: 67, a HCDR3 having at least 90% sequence identity with SEQ ID NO: 68, a light chain complementarity determining region 1 (LCDR1) having at least 90% sequence identity with SEQ ID NO: 69, a LCDR2 having at least 90% sequence identity with SEQ ID NO: 70, and a LCDR3 having at least 90% sequence identity with SEQ ID NO: 71.

45. The antibody or antigen-binding fragment thereof of claim 43 or claim 44, comprising a heavy chain variable region (VH) comprising the amino acid sequence of SEQ ID NO: 72 and a light chain variable region (VL) comprising the amino acid sequence of SEQ ID NO: 73.

46. An antibody or antigen-binding fragment thereof that binds thymic stromal lymphopoietin (TSLP), wherein the antibody or antigen-binding fragment thereof comprises a heavy chain variable region (VH) comprising an amino acid sequence with at least 90% sequence identity to SEQ ID NO: 84 and a light chain variable region (VL) comprising an amino acid sequence with at least 90% sequence identity to SEQ ID NO: 85.

47. The antibody or antigen-binding fragment thereof of claim 46, wherein the antibody or antigen-binding fragment thereof comprises a heavy chain complementarity determining region 1 (HCDR1) having at least 90% sequence identity with SEQ ID NO: 76, a HCDR2 having at least 90% sequence identity with SEQ ID NO: 77, a HCDR3 having at least 90% sequence identity with SEQ ID NO: 78, a light chain complementarity determining region 1 (LCDR1) having at least 90% sequence identity with SEQ ID NO: 79, a LCDR2 having at least 90% sequence identity with SEQ ID NO: 80, and a LCDR3 having at least 90% sequence identity with SEQ ID NO: 81.

48. The antibody or antigen-binding fragment thereof of claim 46 or claim 47, comprising a heavy chain variable region (VH) comprising the amino acid sequence of SEQ ID NO: 84 and a light chain variable region (VL) comprising the amino acid sequence of SEQ ID NO: 85.

49. The antibody of any one of claims 34-48, wherein the antibody further comprises a region capable of binding a second antigen.

50. The antibody of claim 49, wherein the second antigen is thymic stromal lymphopoietin (TSLP), and wherein the region capable of binding a second antigen comprises a a heavy chain variable region (VH) comprising an amino acid sequence with at least 90% sequence identity to SEQ ID NO: 84 and a light chain variable region (VL) comprising an amino acid sequence with at least 90% sequence identity to SEQ ID NO: 85.

51. The antibody of claim 49 or claim 50, wherein the region capable of binding a second antigen comprises a heavy chain complementarity determining region 1 (HCDR1) having at least 90% sequence identity with SEQ ID NO: 76, a HCDR2 having at least 90% sequence identity with SEQ ID NO: 77, a HCDR3 having at least 90% sequence identity with SEQ ID NO: 78, a light chain complementarity determining region 1 (LCDR1) having at least 90% sequence identity with SEQ ID NO: 79, a LCDR2 having at least 90% sequence identity with SEQ ID NO: 80, and a LCDR3 having at least 90% sequence identity with SEQ ID NO: 81.

52. The antibody of any one of claims 49-51, wherein the region capable of binding a second antigen comprises a heavy chain variable region (VH) comprising the amino acid sequence of SEQ ID NO: 84 and a light chain variable region (VL) comprising the amino acid sequence of SEQ ID NO: 85.

53. The antibody or antigen-binding fragment thereof of any one of claims 34-52, wherein the antibody or antigen-binding portion is humanized or chimeric.

54. The antibody or antigen-binding fragment thereof of any one of claims 34-53, wherein the VH region, the VL region, or both the VH and the VL region comprise one or more human framework region amino acid sequences.

55. The antibody or antigen-binding fragment thereof of any one of claims 34-54, wherein the antibody or antigen-binding portion comprises an immunoglobulin constant region.

56. The antibody or antigen-binding fragment thereof of claim 55, wherein the immunoglobulin constant region is an IgG, IgE, IgM, IgD, IgA or IgY constant region.

57. The antibody or antigen-binding fragment thereof of claim 55 or claim 56, wherein the immunoglobulin constant region is an IgG1, IgG2, IgG3, IgG4, IgA1 or IgA2 constant region.

58. The antibody or antigen-binding fragment thereof of any one of claims 55-57, wherein the immunoglobulin constant region is immunologically inert.

59. The antibody or antigen-binding fragment thereof of any one of claims 34-57, wherein the antibody or antigen-binding fragment thereof comprises a human IgG1 constant region comprising a L234A substitution, a L235A substitution, a G237A substitution, a Q311R substitution, a D356E substitution, a L358M substitution, a M428L substitution, a H433K substitution, a N434S substitution, a N434F substitution or a combination thereof, wherein numbering is according to the EU index as in Kabat.

60. The antibody or antigen-binding fragment thereof of any one of claims 34-57, wherein the immunoglobulin constant region comprises a human IgG4 constant region comprising a F234A substitution, a L235A substitution, a G237A substitution, Q311R substitution, a D356E substitution, a L358M substitution, a M428L substitution, a H433K substitution, a N434S substitution, a N434F substitution or a combination thereof, wherein numbering is according to the EU index as in Kabat.

61. The antibody or antigen-binding fragment thereof of claim 59 or claim 60, wherein the human IgG1 constant region or human IgG4 constant region comprises a Q311R substitution, a M428L substitution, and a N434F substitution, wherein numbering is according to the EU index as in Kabat.

62. The antibody or antigen-binding fragment thereof of claim 59 or claim 60, wherein the human IgG1 constant region comprises a M428L substitution, a H433K substitution, and a N434F substitution, wherein numbering is according to the EU index as in Kabat.

63. The antibody or antigen-binding fragment thereof of claim 59 or claim 60, wherein the human IgG1 constant region or human IgG4 constant region comprises a Q311R substitution, a M428L substitution, a H433K substitution, and a N434F substitution, wherein numbering is according to the EU index as in Kabat.

64. The antibody or antigen-binding fragment thereof of any one of claims 34-57, wherein the immunoglobulin constant region is a human IgG1 constant region comprising a L234A substitution, a L235A substitution, a G237A substitution, a D356E substitution, and a L358M substitution, wherein numbering is according to the EU index as in Kabat.

65. The antibody or antigen-binding fragment thereof of any one of claims 34-57, wherein the immunoglobulin constant region is a human IgG4 constant region comprising a F234A substitution, a L235A substitution, a G237A substitution, a D356E substitution, and a L358M substitution, wherein numbering is according to the EU index as in Kabat.

66. The antibody or antigen-binding fragment thereof of any one of claims 34-57, wherein the antibody or antigen-binding fragment thereof comprises a M428L substitution and a N434S substitution, wherein numbering is according to the EU index as in Kabat.

67. The antibody or antigen-binding fragment thereof of any one of claims 34-57, wherein the antibody or antigen-binding fragment thereof comprises a Q311R substitution, a M428L substitution, a H433K substitution, and a N434F substitution, wherein numbering is according to the EU index as in Kabat.

68. The antibody or antigen-binding fragment thereof of any one of claims 34-67, comprising:
a) a heavy chain (HC) comprising the amino acid sequence of SEQ ID NO: 9 and a light chain (LC) comprising the amino acid sequence of SEQ ID NO: 10;
b) a HC comprising the amino acid sequence of SEQ ID NO: 15 and a LC comprising the amino acid sequence of SEQ ID NO: 16;
c) a HC comprising the amino acid sequence of SEQ ID NO: 9 and a LC comprising the amino acid sequence of SEQ ID NO: 19;
d) a HC comprising the amino acid sequence of SEQ ID NO: 9 and a LC comprising the amino acid sequence of SEQ ID NO: 21; or
e) a HC comprising the amino acid sequence of SEQ ID NO: 24 and a LC comprising the amino acid sequence of SEQ ID NO: 21;
f) a HC comprising the amino acid sequence of SEQ ID NO: 26 and a LC comprising the amino acid sequence of SEQ ID NO: 10;
g) a HC comprising the amino acid sequence of SEQ ID NO: 29 and a LC comprising the amino acid sequence of SEQ ID NO: 21;
h) a HC comprising the amino acid sequence of SEQ ID NO: 51 and a LC comprising the amino acid sequence of SEQ ID NO: 52; or
i) a HC comprising the amino acid sequence of SEQ ID NO: 61 and a LC comprising the amino acid sequence of SEQ ID NO: 62;
j) a HC comprising the amino acid sequence of SEQ ID NO: 65 and a LC comprising the amino acid sequence of SEQ ID NO: 62;
k) a HC comprising the amino acid sequence of SEQ ID NO: 74 and a LC comprising the amino acid sequence of SEQ ID NO: 75;
l) a HC comprising the amino acid sequence of SEQ ID NO: 88 and a LC comprising the amino acid sequence of SEQ ID NO: 89; or
m) a single-chain variable fragment linked to an Fc region (scFv-Fc) comprising the amino acid sequence of SEQ ID NO: 90.

69. The antibody or antigen-binding fragment thereof of any one of claims 34-67, comprising a heavy chain (HC) comprising the amino acid sequence of SEQ ID NO: 88 and a light chain (LC) comprising the amino acid sequence of SEQ ID NO: 89; and
a single-chain variable fragment linked to an Fc region (scFv-Fc) comprising the amino acid sequence of SEQ ID NO: 90.

70. The antibody or antigen-binding fragment thereof of any one of claims 54-69, wherein the immunoglobulin constant region comprises (i) any one of SEQ ID NO: 33, 35, 95-98, and 128-132; and (ii) SEQ ID NO: 36.

71. The antibody or antigen-binding fragment thereof of any one of claims 34-70, wherein the antibody has increased affinity for FcRn compared to a comparator antibody.

72. The antibody or antigen-binding fragment thereof of claim 71, wherein the comparator antibody is RVT-3101, AVTX-009, APG-777, APG-777×AIO-001, or CALY-002.

73. The antibody or antigen-binding fragment thereof of any one of claims 34-72, wherein the antibody or antigen-binding fragment is an Fab, an Fab′, an F(ab′)₂, an Fv, an scFv, a maxibody, a minibody, a diabody, a triabody, a tetrabody, or a bis-scFv.

74. The antibody or antigen-binding fragment thereof of any one of claims 34-73, wherein the antibody or antigen-binding portion is bispecific.

75. An immunoconjugate comprising the antibody or antigen-binding fragment thereof of any one of claims 34-74, linked to a therapeutic agent.

76. A pharmaceutical composition comprising the antibody or antigen-binding fragment thereof of any one of claims 34-74, or the immunoconjugate of claim 75, and a pharmaceutically acceptable carrier, diluent or excipient.

77. A nucleic acid molecule encoding the antibody or antigen-binding fragment thereof of any one of claims 34-74.

78. An expression vector comprising the nucleic acid molecule of claim 77.

79. A recombinant host cell comprising the antibody or antigen-binding fragment thereof of any one of claims 34-74, the nucleic acid molecule of claim 77, or the vector of claim 78.

80. A recombinant host cell expressing the antibody or antigen-binding fragment thereof of any one of claims 34-74.

81. A method of producing an antibody or an antigen-binding fragment thereof, the method comprising:
(i) culturing the recombinant host cell of claim 79 or 80 under conditions whereby the nucleic acid molecule is expressed, thereby producing the antibody or antigen-binding portion; and
(ii) isolating the antibody or antigen-binding portion from the host cell or culture.

82. A method for reducing death recenter 3 (DR3) signaling in a cell, wherein the method comprises contacting the cell with the antibody or antigen-binding portion of any one of claims 34-74 or the immunoconjugate of claim 75.

83. A method for reducing IL4/IL13 signaling in a cell, wherein the method comprises contacting the cell with the antibody or antigen-binding portion of any one of claims 34-74 or the immunoconjugate of claim 75.

84. A method for reducing IL6 release by a cell, wherein the method comprises contacting the cell with the antibody or antigen-binding portion of any one of claims 34-74 or the immunoconjugate of claim 75.

85. A method for reducing release of one or more pro-inflammatory cytokines by a cell, wherein the method comprises contacting the cell with the antibody or antigen-binding portion of any one of claims 34-74 or the immunoconjugate of claim 75.

86. A method for reducing IL6 release by a cell, wherein the method comprises contacting the cell with the antibody or antigen-binding portion of any one of claims 34-74 or the immunoconjugate of claim 75.

87. The method of claim 82, wherein the method results in reduced proliferation of the cell.

88. The method of claim 82 or claim 83, wherein the method results in reduced production of a cytokine, preferably TNF-α.

89. The antibody or antigen-binding portion of any one of claims 34-74, the immunoconjugate of claim 75, or the pharmaceutical composition of claim 76, for use as a medicament.


SEQUENCE LISTING (137 sequences)

SEQ ID NO: 1 - protein, 10 aa, synthetic construct
GYDFTYYGIS

SEQ ID NO: 2 - protein, 17 aa, synthetic construct
WISPYNGNTHYAQKLQG

SEQ ID NO: 3 - protein, 15 aa, synthetic construct
ENYYGSGAYRGGMDV

SEQ ID NO: 4 - protein, 11 aa, synthetic construct
RASQSVSSYLA

SEQ ID NO: 5 - protein, 7 aa, synthetic construct
DASNRAT

SEQ ID NO: 6 - protein, 9 aa, synthetic construct
QQRSSWPWT

SEQ ID NO: 7 - protein, 124 aa, synthetic construct
QVQLVQSGAEVKKPGASVKVSCKASGYDFTYYGISWVRQAPGQGLEWMGWISPYNGNTHYAQKLQGRVTMTTDTSTRTAYMELRSLRSDDTAVYYCARENYYGSGAYRGGMDVWGQGTTVTVSS

SEQ ID NO: 8 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSSWPWTFGQGTKVEIK

SEQ ID NO: 9 - protein, 453 aa, synthetic construct
QVQLVQSGAEVKKPGASVKVSCKASGYDFTYYGISWVRQAPGQGLEWMGWISPYNGNTHYAQKLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSSWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 17 aa, synthetic construct
WISPYNGNTHYARMLQG

SEQ ID NO: 12 - protein, 9 aa, synthetic construct
QQRSNWPWT

SEQ ID NO: 13 - protein, 124 aa, synthetic construct
QVQLVQSGAEVKKPGASVKVSCKASGYDFTYYGISWVRQAPGQGLEWMGWISPYNGNTHYARMLQGRVTMTTDTSTRTAYMELRSLRSDDTAVYYCARENYYGSGAYRGGMDVWGQGTTVTVSS

SEQ ID NO: 14 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 15 - protein, 453 aa, synthetic construct
QVQLVQSGAEVKKPGASVKVSCKASGYDFTYYGISWVRQAPGQGLEWMGWISPYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 7 aa, synthetic construct
DASTRAT

SEQ ID NO: 18 - protein, 107 aa, synthetic construct
EIVLTQSPATLSLSPGERATLSCRASQSVSSYLAWYQQKPGQAPRLLIYDASTRATGIPARFSGSGSGTDFTLTISSLEPEDFAVYYCQQRSSWPWTFGQGTKVEIK

SEQ ID NO: 19 - protein, 214 aa, synthetic construct
EIVLTQSPATLSLSPGERATLSCRASQSVSSYLAWYQQKPGQAPRLLIYDASTRATGIPARFSGSGSGTDFTLTISSLEPEDFAVYYCQQRSSWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 107 aa, synthetic construct
EIVLTQSPATLSLSPGERATLSCRASQSVSSYLAWYQQKPGQAPRLLIYDASTRATGIPARFSGSGSGTDFTLTISSLEPEDFAVYYCQQRSNWPWTFGQGTKVEIK

SEQ ID NO: 21 - protein, 214 aa, synthetic construct
EIVLTQSPATLSLSPGERATLSCRASQSVSSYLAWYQQKPGQAPRLLIYDASTRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 17 aa, synthetic construct
WISPYNGNTHYAQMLQG

SEQ ID NO: 23 - protein, 124 aa, synthetic construct
QVQLVQSGAEVKKPGASVKVSCKASGYDFTYYGISWVRQAPGQGLEWMGWISPYNGNTHYAQMLQGRVTMTTDTSTRTAYMELRSLRSDDTAVYYCARENYYGSGAYRGGMDVWGQGTTVTVSS

SEQ ID NO: 24 - protein, 453 aa, synthetic construct
QVQLVQSGAEVKKPGASVKVSCKASGYDFTYYGISWVRQAPGQGLEWMGWISPYNGNTHYAQMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 5 aa, synthetic construct
YYGIS

SEQ ID NO: 26 - protein, 453 aa, synthetic construct
QVQLVQSGAEVKKPGASVKVSCKASGYDFTYYGISWVRQAPGQGLEWMGWISPYNGNTHYAQKLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 27 - protein, 15 aa, synthetic construct
EDYYGSGSYRGGMDV

SEQ ID NO: 28 - protein, 124 aa, synthetic construct
QVQLVQSGAEVKKPGASVKVSCKASGYDFTYYGISWVRQAPGQGLEWMGWISPYNGNTHYAQKLQGRVTMTTDTSTRTAYMELRSLRSDDTAVYYCAREDYYGSGSYRGGMDVWGQGTTVTVSS

SEQ ID NO: 29 - protein, 453 aa, synthetic construct
QVQLVQSGAEVKKPGASVKVSCKASGYDFTYYGISWVRQAPGQGLEWMGWISPYNGNTHYAQKLQGRVTMTTDTSTRTAYMELRSLRSDDTAVYYCAREDYYGSGSYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 30 - protein, 17 aa, synthetic construct
WISTYNGNTHYARMLQG

SEQ ID NO: 31 - protein, 124 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 32 - protein, 453 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 35 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 36 - protein, 107 aa, synthetic construct [sequence omitted]

SEQ ID NO: 37 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 38 - protein, 251 aa, Macaca mulatta
MAEDLGLSFGETASVEMLPEHGSCRPKARSSSACWALTCCLVLLPFLAGLTTYLLVSQLRAQGEACVQLQDLKGQEFAPSHQQVYAPLRADGDKPRAHLTVVRQTPTQHLKNQFPALHWEHELGLAFTKNRMNYTNKFLLIPESGDYFVYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 39 - protein, 252 aa, Mus musculus
MAEELGLGFGEGVPVEVLPEGCRHRPEARAGLAARSKACLALTCCLLSFPILAGLSTLLMAGQLRVPGKDCMLRAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITMVITKVADSYPEPARLLTGSKSVCEISNNWFQSLYLGATFSLEEGDRLMVNVSDISLVDYTKEDKTFFGAFLL

SEQ ID NO: 40 - protein, 222 aa, synthetic construct
TCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 41 - protein, 222 aa, synthetic construct
TCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 42 - protein, 222 aa, synthetic construct
TCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 5 aa, synthetic construct
DHYMS

SEQ ID NO: 44 - protein, 17 aa, synthetic construct
YISSSGSTIYYADSVKG

SEQ ID NO: 45 - protein, 14 aa, synthetic construct
EYDSSGYYYVGVDF

SEQ ID NO: 46 - protein, 11 aa, synthetic construct
RASQSISSYLN

SEQ ID NO: 47 - protein, 7 aa, synthetic construct
GASSDQS

SEQ ID NO: 48 - protein, 9 aa, synthetic construct
QQGYYFPPT

SEQ ID NO: 49 - protein, 123 aa, synthetic construct
EVQLVESGGGLVQPGGSLRLSCAASGFTFSDHYMSWIRQAPGKGLEWVSYISSSGSTIYYADSVKGRFTISRDNAKNSLYLQMNSLRAEDTAVYYCAREYDSSGYYYVGVDFWGQGTLVTVSS

SEQ ID NO: 50 - protein, 107 aa, synthetic construct
DIQMTQSPSSLSASVGDRVTITCRASQSISSYLNWYQQKPGKAPKLLIYGASSDQSGVPSRFSGSGSGTDFTLTISSLQPEDFATYYCQQGYYFPPTFGGGTKVEIK

SEQ ID NO: 51 - protein, 449 aa, synthetic construct
EVQLVESGGGLVQPGGSLRLSCAASGFTFSDHYMSWIRQAPGKGLEWVSYISSSGSTIYYADSVKGRFTISRDNAKNSLYLQMNSLRAEDTAVYYCAREYDSSGYYYVGVDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALKFHYTQKSLSLSLG

SEQ ID NO: 52 - protein, 214 aa, synthetic construct
DIQMTQSPSSLSASVGDRVTITCRASQSISSYLNWYQQKPGKAPKLLIYGASSDQSGVPSRFSGSGSGTDFTLTISSLQPEDFATYYCQQGYYFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 5 aa, synthetic construct
AYSVN

SEQ ID NO: 54 - protein, 16 aa, synthetic construct
MIWGDGKIVYNSALKS

SEQ ID NO: 55 - protein, 10 aa, synthetic construct
DGYYPYFMDN

SEQ ID NO: 56 - protein, 15 aa, synthetic construct
RASKSVDSYGNSFMH

SEQ ID NO: 57 - protein, 7 aa, synthetic construct
LASNLES

SEQ ID NO: 58 - protein, 9 aa, synthetic construct
QQNNEDPRT

SEQ ID NO: 59 - protein, 118 aa, synthetic construct
EVQLQESGPGLVKPSETLSLTCTVSGFSLNAYSVNWIRQPPGKGLEWLGMIWGDGKIVYNSALKSRLTISKDSSKNQVSLKLSSVTAADTAVYYCAGDGYYPYFMDNWGQGTTVTVSS

SEQ ID NO: 60 - protein, 111 aa, synthetic construct
DIQLTQSPSSLSASVGDRVTITCRASKSVDSYGNSFMHWYQQKPGKAPKLLIYLASNLESGVPSRFSGSGSGTDFTLTISSLQPEDFATYYCQQNNEDPRTFGGGTKVEIK

SEQ ID NO: 61 - protein, 447 aa, synthetic construct
EVQLQESGPGLVKPSETLSLTCTVSGFSLNAYSVNWIRQPPGKGLEWLGMIWGDGKIVYNSALKSRLTISKDSSKNQVSLKLSSVTAADTAVYYCAGDGYYPYFMDNWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 62 - protein, 218 aa, synthetic construct
DIQLTQSPSSLSASVGDRVTITCRASKSVDSYGNSFMHWYQQKPGKAPKLLIYLASNLESGVPSRFSGSGSGTDFTLTISSLQPEDFATYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 10 aa, synthetic construct
DGYYPYFMDN

SEQ ID NO: 64 - protein, 118 aa, synthetic construct
EVQLQESGPGLVKPSETLSLTCTVSGFSLNAYSVNWIRQPPGKGLEWLGMIWGDGKIVYNSALKSRLTISKDSSKNQVSLKLSSVTAADTAVYYCAGDGYYPYAMDGWGQGTTVTVSS

SEQ ID NO: 65 - protein, 447 aa, synthetic construct
EVQLQESGPGLVKPSETLSLTCTVSGFSLNAYSVNWIRQPPGKGLEWLGMIWGDGKIVYNSALKSRLTISKDSSKNQVSLKLSSVTAADTAVYYCAGDGYYPYAMDGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 66 - protein, 5 aa, synthetic construct
NYAMS

SEQ ID NO: 67 - protein, 17 aa, synthetic construct
TISRGGDYHYYPDSVKG

SEQ ID NO: 68 - protein, 13 aa, synthetic construct
RVSMITGGWAHDY

SEQ ID NO: 69 - protein, 16 aa, synthetic construct
RSSQSIVDITGNTYLE

SEQ ID NO: 70 - protein, 7 aa, synthetic construct
KVFNRFS

SEQ ID NO: 71 - protein, 9 aa, synthetic construct
FQDSFVPYT

SEQ ID NO: 72 - protein, 122 aa, synthetic construct
EVQLVESGGGLVQPGGSLRLSCAASEFTFSNYAMSWVRQAPGKGLEWVATISRGGDYHYYPDSVKGRFTISRDNSKNTLYLQMNSLRAEDTAVYYCARRVSMITGGWAHDYWGQGTLVTVSS

SEQ ID NO: 73 - protein, 112 aa, synthetic construct
DVVMTQSPLSLPVTLGQPASISCRSSQSIVDITGNTYLEWYQQRPGQSPRLLIYKVFNRFSGVPDRFSGSGSGTDFTLKISRVEAEDVGVYYCFQDSFVPYTFGQGTKLEIK

SEQ ID NO: 74 - protein, 452 aa, synthetic construct
EVQLVESGGGLVQPGGSLRLSCAASEFTFSNYAMSWVRQAPGKGLEWVATISRGGDYHYYPDSVKGRFTISRDNSKNTLYLQMNSLRAEDTAVYYCARRVSMITGGWAHDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKFHYTQKSLSLSPGK

SEQ ID NO: 75 - protein, 219 aa, synthetic construct
DVVMTQSPLSLPVTLGQPASISCRSSQSIVDITGNTYLEWYQQRPGQSPRLLIYKVFNRFSGVPDRFSGSGSGTDFTLKISRVEAEDVGVYYCFQDSFVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 5 aa, synthetic construct
NYLIE

SEQ ID NO: 77 - protein, 17 aa, synthetic construct
VIDPGVGDTNYNENFKG

SEQ ID NO: 78 - protein, 10 aa, synthetic construct
EDNPGTAFDY

SEQ ID NO: 79 - protein, 11 aa, synthetic construct
KASQSVSSDVT

SEQ ID NO: 80 - protein, 7 aa, synthetic construct
YVSEHYT

SEQ ID NO: 81 - protein, 9 aa, synthetic construct
QQHHRFPLT

SEQ ID NO: 82 - protein, 10 aa, synthetic construct
DGYYPYAMRN

SEQ ID NO: 83 - protein, 15 aa, synthetic construct
RASKSVDSYGNSFMH

SEQ ID NO: 84 - protein, 119 aa, synthetic construct
EVQLVQSGAEVKKPGSSVKVSCKASGYTFSNYLIEWVRQAPGQGLEWIGVIDPGVGDTNYNENFKGRATLTADKSTSTAYIELSSLRSEDTAVYYCAREDNPGTAFDYWGQGTTVTVSS

SEQ ID NO: 85 - protein, 107 aa, synthetic construct
SIVMTQTPLSLSVTPGQPASISCKASQSVSSDVTWYLQKPGQSPQLLIYYVSEHYTGVPDRFSGSGYGTDFTLKISRVEAEDVGVYYCQQHHRFPLTFGQGTKLEIK

SEQ ID NO: 86 - protein, 118 aa, synthetic construct
EVQLQESGPGLVKPSETLSLTCTVSGFSLNAYSVNWIRQPPGKCLEWLGMIWGDGKIVYNSALKSRLTISKDSSKNQVSLKLSSVTAADTAVYYCAGDGYYPYAMRNWGQGTTVTVSS

SEQ ID NO: 87 - protein, 111 aa, synthetic construct
DIQLTQSPSSLSASVGDRVTITCRASKSVDSYGNSFMHWYQQKPGKAPKLLIYLASNLESGVPSRFSGSGSGTDFTLTISSLQPEDFATYYCQQNNEDPRTFGCGTKVEIK

SEQ ID NO: 88 - protein, 449 aa, synthetic construct
EVQLVQSGAEVKKPGSSVKVSCKASGYTFSNYLIEWVRQAPGQGLEWIGVIDPGVGDTNYNENFKGRATLTADKSTSTAYIELSSLRSEDTAVYYCAREDNPGTAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALKFHYTQKSLSLSPGK

SEQ ID NO: 89 - protein, 214 aa, synthetic construct
SIVMTQTPLSLSVTPGQPASISCKASQSVSSDVTWYLQKPGQSPQLLIYYVSEHYTGVPDRFSGSGYGTDFTLKISRVEAEDVGVYYCQQHHRFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 481 aa, synthetic construct
DIQLTQSPSSLSASVGDRVTITCRASKSVDSYGNSFMHWYQQKPGKAPKLLIYLASNLESGVPSRFSGSGSGTDFTLTISSLQPEDFATYYCQQNNEDPRTFGCGTKVEIKGGGGSGGGGSGGGGSGGGGSEVQLQESGPGLVKPSETLSLTCTVSGFSLNAYSVNWIRQPPGKCLEWLGMIWGDGKIVYNSALKSRLTISKDSSKNQVSLKLSSVTAADTAVYYCAGDGYYPYAMRNWGQGTTVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALKFHYTQKSLSLSPGK

SEQ ID NO: 91 - protein, 453 aa, synthetic construct
QVQLVQSGAEVKKPGASVKVSCKASGYDFTYYGISWVRQAPGQGLEWMGWISPYNGNTHYAQKLQGRVTMTTDTSTRTAYMELRSLRSDDTAVYYCAREDYYGSGSYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 92 - protein, 453 aa, synthetic construct
QVQLVQSGAEVKKPGASVKVSCKASGYDFTYYGISWVRQAPGQGLEWMGWISPYNGNTHYAQKLQGRVTMTTDTSTRTAYMELRSLRSDDTAVYYCAREDYYGSGSYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 93 - protein, 453 aa, synthetic construct
QVQLVQSGAEVKKPGASVKVSCKASGYDFTYYGISWVRQAPGQGLEWMGWISPYNGNTHYAQKLQGRVTMTTDTSTRTAYMELRSLRSDDTAVYYCAREDYYGSGSYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHFHYTQKSLSLSPG

SEQ ID NO: 94 - protein, 453 aa, synthetic construct
QVQLVQSGAEVKKPGASVKVSCKASGYDFTYYGISWVRQAPGQGLEWMGWISPYNGNTHYAQKLQGRVTMTTDTSTRTAYMELRSLRSDDTAVYYCAREDYYGSGSYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKNHYTQKSLSLSPG

SEQ ID NO: 95 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 96 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 97 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHFHYTQKSLSLSPG

SEQ ID NO: 98 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKNHYTQKSLSLSPG

SEQ ID NO: 99 - protein, 222 aa, synthetic construct
TCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 100 - protein, 222 aa, synthetic construct
TCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 101 - protein, 222 aa, synthetic construct
TCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHFHYTQKSLSLSPG

SEQ ID NO: 102 - protein, 222 aa, synthetic construct
TCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKNHYTQKSLSLSPG

SEQ ID NO: 103 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 104 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 222 aa, synthetic construct
PCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 108 - protein, 222 aa, synthetic construct
TCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 14 aa, synthetic construct
EADSSGYYYVGVDV

SEQ ID NO: 112 - protein, 10 aa, synthetic construct
DGYYPYAMDN

SEQ ID NO: 113 - protein, 10 aa, synthetic construct
EDNTGTAFDY

SEQ ID NO: 114 - protein, 17 aa, synthetic construct
TISRGGDYTYYPDSVKG

SEQ ID NO: 115 - protein, 13 aa, synthetic construct
RVSMITGGWAMDY

SEQ ID NO: 116 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 117 - protein, 269 aa, Macaca mulatta
MAEVPELASEMMAYYSGNEDDLFFDVDGPKQMKCSFQDLDLCPLGGGIQLQISHEHYNEGFRQAVSVVVAMEKLRKMLVPCPQIFQDNDLSTLIPFIFEEEPVFLDTRNNDACVHDAPVRSLHCTLRDAQLKSLVMSGPYELKALHLQGQDLEQQVVFSMSFVQGEESNDKIPVALGLKAKNLYLSCVLKDDKPTLQLESVDPKNYPKKKMEKRFVFNKIEINNKLEFESAQFPNWYISTSQAENMPVFLGGTRGGQDITDFTMQFVSS

SEQ ID NO: 118 - protein, 269 aa, Mus musculus
MATVPELNCEMPPFDSDENDLFFEVDGPQKMKGCFQTFDLGCPDESIQLQISQQHINKSFRQAVSLIVAVEKLWQLPVSFPWTFQDEDMSTFFSFIFEEEPILCDSWDDDDNLLVCDVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 119 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 120 - protein, 132 aa, Macaca mulatta
MALLLTTVIALTCLGGFASPSPVPRSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGRFN

SEQ ID NO: 121 - protein, 131 aa, Mus musculus
MALWVTAVLALACLGGLAAPGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 122 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 123 - protein, 159 aa, Macaca mulatta
MKSLGQSKKEEVSFRKFFIFQLVGLVLTYDFTNCDFEKIEADYLRTISKDLITYMSGTKSTDFNNTVSCSNRPHCLTEIQSLTFNPTPRCASLAKEMFARKTKATLALWCPGYSETQINATQAMKKRRKRKVTTNKCLEQVSQLLGLWRRFIRTLLKKQ

SEQ ID NO: 124 - protein, 140 aa, Mus musculus
MVLLRSLFILQVLVRMGLTYNFSNCNFTSITKIYCNIIFHDLTGDLKGAKFEQIEDCESKPACLLKIEYYTLNPIPGCPSLPDKTFARRTREALNDHCPGYPETERNDGTQEMAQEVQNICLNQTSQILRLWYSFMQSPE

SEQ ID NO: 125 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 126 - protein, 162 aa, Macaca mulatta
MRISKPHLRSVSIQCYLCLLLNSHFLTEAGIHVFILGCFSAGLPKTEANWVNVISDLKKIEDLIQSMHIDATLYTESDVHPSCKVTAMKCFLLELQVISHESGDTDIHDTVENLIILANNILSSNGNITESGCKECEELEEKNIKEFLQSFVHIVQMFINTS

SEQ ID NO: 127 - protein, 162 aa, Mus musculus
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 128 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALKFHYTQKSLSLSLG

SEQ ID NO: 129 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 130 - protein, 329 aa, synthetic construct; identical to SEQ ID NO: 129

SEQ ID NO: 131 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALKFHYTQKSLSLSPGK

SEQ ID NO: 132 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKFHYTQKSLSLSPGK

SEQ ID NO: 133 - protein, 222 aa, synthetic construct
PCPPCPAPEAAGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALKFHYTQKSLSLSLG

SEQ ID NO: 134 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 135 - protein, 222 aa, synthetic construct; identical to SEQ ID NO: 134

SEQ ID NO: 136 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALKFHYTQKSLSLSPGK

SEQ ID NO: 137 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALKFHYTQKSLSLSPGK